(19) United States
(12) Patent Application Publication
(10) Pub. No.: US 2025/0270569 A1
(43) Pub. Date: Aug. 28, 2025

(54) CELL-ASSOCIATED HETEROLOGOUS FOOD AND/OR FEED ENZYMES

(71) Applicant: Danstar Ferment AG, Zug (CH)

(72) Inventors: Aaron Argyros, Lebanon, NH (US); Michelle Oeser, Croydon, NH (US); Erin Wiswall, Danbury, NH (US); Janet Fisher, Enfield, NH (US); Johannes Van Eijk, Longueuil (CA); J. Kevin Kraus, Tenafly, NJ (US); Kevin Wenger, Hanover, NH (US); Brooks Henningsen, Salisbury, NH (US); Ryan Skinner, South Royalton, VT (US)

(21) Appl. No.: 19/198,857

(22) Filed: May 5, 2025

Related U.S. Application Data
(63) Continuation of application No. 16/493,245, filed on Sep. 11, 2019, now Pat. No. 12,344,847.
(60) Provisional application No. 62/470,568, filed on Mar. 13, 2017.
(60) Provisional application No. 62/625,751, filed on Feb. 2, 2018.

(51) Int. Cl. C12N 15/81; A21D 8/04; A23K 10/18; A23K 20/189; A23L 33/14; C12N 1/18; C12N 9/16; C12N 9/30; C12N 9/34

(52) U.S. Cl. CPC C12N 15/81; A21D 8/047; A23K 10/18; A23K 20/189; A23L 33/14; C12N 1/18; C12N 9/16; C12N 9/242; C12N 9/2428; C12Y 302/01133

(57) ABSTRACT

The present disclosure concerns recombinant yeast host cells expressing cell-associated heterologous food and/or feed enzymes which are expressed during the propagation phase of the recombinant yeast hosts cells. The recombinant yeast host cells can be used in a subsequent production process to make food and/or feed products, for example, baked products.

### CROSS-REFERENCE TO RELATED APPLICATIONS

The present application is a continuation of U.S. patent application Ser. No. 16/493,245, filed on Sep. 11, 2019, which is a U.S. national phase application of PCT/IB2018/051670 filed on Mar. 13, 2017, which claims priority from U.S. provisional patent application 62/470,568 filed on Mar. 13, 2017, and U.S. provisional patent application 62/625,751, filed Feb. 2, 2018, all of which are herewith incorporated by reference in their entirety.

### REFERENCE TO AN ELECTRONIC SEQUENCE LISTING

The contents of the electronic sequence listing (580127_407C1_SL.xml; Size: 173,170 bytes; and Date of Creation: May 5, 2025) is herein incorporated by reference in its entirety.

### TECHNOLOGICAL FIELD

The present disclosure relates to a recombinant yeast host cell expressing cell-associated enzymes and acting as a source of enzyme activity in the production of food and/or feed, including those for the production of baked products.

### BACKGROUND

Commercial yeast and commercial enzymes are commonly used to produce food and feed such as, for example, bread and other yeast-leavened baked products. Commercial enzymes are also used without yeast for making food and feed, for example in chemically leavened and unleavened baked products such as cakes and flatbreads. Baker's yeasts are produced from strains of Saccharomyces cerevisiae by fed-batch propagation and supplied in fresh or dried form. Food and feed enzymes (such as baking, brewery and feed enzymes) are produced from various plants, bacteria and fungi, often enhanced by genetic modification. The bacterial and fungal enzymes are usually expressed and excreted from the production organism during sterile batch fermentation, separated, purified, concentrated, dried and supplied as granulated powders.

Baking enzymes can be added to the flour, dough, or batter that is used to prepare the baked products. They act on carbohydrates, proteins and lipids during mixing, proofing and baking to facilitate processing and improve the appearance, texture and keeping quality of the finished product. For example, maltogenic alpha-amylase (MAA) is added to the dough or batter during the production of breads, cakes, and other baked products. It acts on the amylopectin part of wheat starch in the oven to inhibit starch retrogradation in the finished product and slow the rate of firming.

Food and feed enzymes in general, and baking enzymes in particular, are a significant part of the cost of producing baked products. As such, there is an incentive to lower the utilization or render obsolete the use of exogenous purified food and feed enzymes such as baking enzymes in the process for making same.

### BRIEF SUMMARY

The present disclosure provides recombinant yeast host cells which have been genetically engineered to express one or more cell-associated heterologous food and/or feed enzyme as well as process for using them or products derived from them to make food and/or feed products.

According to a first aspect, the present disclosure provides a recombinant yeast host cell having an heterologous nucleic acid molecule encoding a cell-associated heterologous food and/or feed enzyme. The heterologous nucleic acid molecule is operatively associated with an heterologous promoter allowing the expression of the heterologous nucleic acid molecule during propagation. In an embodiment, the heterologous nucleic acid molecule allows the intracellular expression of the heterologous food and/or feed enzyme. In another embodiment, the heterologous nucleic acid molecule allows the expression of a membrane-associated heterologous food and/or feed enzyme. For example, the heterologous nucleic acid molecule can allow the expression of a tethered heterologous food and/or feed enzyme. In an embodiment, the tethered heterologous food and/or feed enzyme is a chimeric protein of formula (I):

$$( {NH}_{2} ) FFE - L - TT ( COOH ) \qquad ( I )$$

wherein FFE is the food and/or feed enzyme; L is present or absent and is an amino acid linker; TT is an amino acid tethering moiety for associating the food and/or feed enzyme to a cell wall of the recombinant yeast host cell; and “-” is an amide linkage. In the chimeric protein of formula (I), (NH₂) indicates the location of the amino terminus of the chimeric protein and (COOH) indicates the location of the carboxyl terminus of the chimeric protein.

In another embodiment, the heterologous food and/or feed enzyme is a chimeric protein of formula (II):

$$( {NH}_{2} ) TT - L - FFE ( COOH ) \qquad ( II )$$

wherein FFE is the food and/or feed enzyme; L is present or absent and is an amino acid linker; TT is an amino acid tethering moiety for associating the food and/or feed enzyme to a cell wall of the recombinant yeast host cell; and “-” is an amide linkage. In the chimeric protein of formula (II), (NH₂) indicates the location of the amino terminus of the chimeric protein and (COOH) indicates the location of the carboxyl terminus of the chimeric protein. In an embodiment, the heterologous nucleic acid molecule encodes the heterologous food enzyme, such as, for example, an alpha-acetolactate decarboxylase, an aminopeptidase, an amylase, a maltogenic amylase, an asparaginase, a bromelain, a carboxypeptidase, a catalase, a cellulase, a chymosin, a cyprosin, a ficin, a glucoamylase, a glucanase, a glucose oxidase, a glucose isomerase, an hemicellulase, an hexose oxidase, an inulinase, an invertase, a lactase, a lipase, a lipoxidase, a lysozyme, a mannanase, a milk coagulating enzyme, a pancreatin, a papain, a pectinase, a pentosanase, a pepsin, a phospholipase, a peroxidase, a protease, a pullulanase, a rennet, a transglutaminase, a trypsin, a urease and/or a xylanase. In still another embodiment, the heterologous food enzyme is an heterologous baking enzyme, such as, for example, an amylolytic enzyme, a cellulase, an hemicellulases, an oxidase, an asparaginase or a lipase. In yet another embodiment, the heterologous food and/or feed enzyme is an amylolytic enzyme, such as, for example, a maltogenic alpha-amylase, a glucoamylase, an alpha-amylase or a fungal amylase. In still another embodiment, the heterologous food and/or feed enzyme is an oxidase such as, for example, a glucose oxidase. In another embodiment, the heterologous nucleic acid molecule encodes the heterologous feed enzyme, such as, for example, a phytase, a beta-glucanase, a xylanase, an alpha-galactosidase, a protease, an amylase, a lipase, a mannanase, a cellulase, an hemicellulase and/or a pectinase. In still yet another embodiment, the heterologous feed enzyme is the phytase. In yet another embodiment of the chimeric protein, L is present and can comprise, for example, one or more G₄S (SEQ ID NO: 41) motifs and/or one or more EA₂K (SEQ ID NO: 100) or EA₃K (SEQ ID NO: 101) motifs. In a further embodiment, TT comprises a transmembrane domain, a variant or a fragment thereof. For example, TT can be from a FLO1 protein. For example, TT can have the amino acid sequence of SEQ ID NO: 14, be a variant of the amino acid sequence of SEQ ID NO: 14 or be a fragment of the amino acid sequence SEQ ID NO: 14. In another embodiment, TT can be modified by a post-translation mechanism to have a glycosylphosphatidylinositol (GPI) anchor. For example, TT can be from a SED1 protein, a TIR1 protein, a CWP2 protein, a CCW12 protein, a SPI1 protein, a PST1 protein or a combination of a AGA1 and a AGA2 protein. In a specific embodiment, TT is from the SPI1 protein and can have, for example, the amino acid sequence of SEQ ID NO: 74, can be a variant of the amino acid sequence of SEQ ID NO: 74 or can be a fragment of the amino acid sequence SEQ ID NO: 74. In a further embodiment, TT can be a fragment of the SPI protein an can have the amino acid sequence of SEQ ID NO: 76, 78, 80 or 82; be a variant of the amino acid sequence of SEQ ID NO: 76, 78, 80 or 82 or be a fragment of the amino acid sequence of SEQ ID NO: 76, 78, 80 or 82. In another specific embodiment, TT is from the CCW12 protein and can have, for example, the amino acid sequence of SEQ ID NO: 84, can be a variant of the amino acid sequence of SEQ ID NO: 84 or can be a fragment of the amino acid sequence of SEQ ID NO: 84. In yet a further embodiment, TT can be a fragment of the CCW12 protein and can have the amino acid sequence of SEQ ID NO: 86, 88, 90 or 92; be a variant of the amino acid sequence of SEQ ID NO: 86, 88, 90 or 92 or be a fragment of the amino acid sequence of SEQ ID NO: 86, 88, 90 or 92. In another embodiment, TT is from the combination of the AGA1 protein and the AGA2 protein and can have, for example, the amino acid sequence of SEQ ID NO: 24, is a variant of the amino acid sequence of SEQ ID NO: 24, is a fragment of the amino acid sequence of SEQ ID NO: 24, has the amino acid sequence of SEQ ID NO: 26, is a variant of the amino acid sequence of SEQ ID NO: 26 or is a fragment of the amino acid sequence of SEQ ID NO: 26. In a further embodiment, the promoter is a native or an heterologous promoter. For example, the heterologous promoter can comprise the promoter the tdh1 gene, the hor7 gene, the hsp150 gene, the hxt7 gene, the gpm1 gene, the pgk1 gene and/or the stl1 gene. The heterologous promoter can comprise, for example, the promoter from the tdh1 gene and/or from the hor7 gene. In some embodiments, the heterologous nucleic acid molecule is operatively associated with a terminator which can be, for example, a native or an heterologous terminator. In some embodiments, the heterologous terminator comprises a terminator from the dit1 gene, the adh3 gene, the idp1 gene, the gpm1 gene, the pma1 gene, the tdh3 gene, the hxt2 gene and/or the ira2 gene. The heterologous terminator can comprise, for example, the terminator from the dit1 gene, from the adh3 gene and/or from the idp1 gene. In an embodiment, the membrane-associated heterologous polypeptide has an heterologous signal peptide, such as, for example, the heterologous signal peptide is from an invertase protein, an AGA2 protein or a fungal amylase. In an embodiment, the heterologous signal peptide is from the invertase protein and can have the amino acid sequence of SEQ ID NO: 68, is a variant of the amino acid sequence of SEQ ID NO: 68 or is a fragment of the amino acid sequence of SEQ ID NO: 68. In still another embodiment, the heterologous signal peptide is from the AGA2 protein and can have the amino acid sequence of SEQ ID NO: 69, is a variant of the amino acid sequence of SEQ ID NO: 69 or is a fragment of the amino acid sequence of SEQ ID NO: 69. In still another embodiment, the heterologous signal peptide is from the fungal amylase and can have the amino acid sequence of SEQ ID NO: 107, is a variant of the amino acid sequence of SEQ ID NO: 107 or is a fragment of the amino acid sequence of SEQ ID NO: 107. In some embodiments, the recombinant yeast host cell can be from the genus Saccharomyces sp. In some further embodiments, the recombinant yeast host cell can be from the species Saccharomyces cerevisiae.

According to a second aspect, the present disclosure provides an additive comprising the food and/or feed enzyme described herein. The additive can comprise or consist essentially of a yeast composition having the recombinant yeast host cell as described herein. In an embodiment, the yeast composition can be provided in a live or inactivated form. The additive can comprise or consist essentially of a yeast product obtained from the recombinant yeast host cell described herein. In an embodiment, the yeast product can be a substantially purified food and/or feed enzyme, a yeast extract or a yeast fraction. The additive can be used as a food additive and/or a feed additive. In an embodiment, the food additive can be a dough conditioner.

According to a fourth aspect, the present disclosure provides a process for making a food or a feed product. The process comprising including the recombinant yeast host cell described herein or the additive described herein in the food or the feed product. In an embodiment, the process further comprises fermenting the food or the feed product in the presence of the recombinant yeast host cells and/or the additive. In another embodiment, the process further comprises baking the food or the feed product to provide a baked product. In such embodiment, the process can be used for extending the shelf-life of the baked product and/or for improving the organoleptic properties of the baked product. In yet another embodiment, the baked product is a bread. In an embodiment, the process can be used to make a food product. In still another embodiment, the process can be used to make a feed product.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A and 1B provide the maltogenic amylase (MAA) enzyme activity measured in (FIG. 1A) yeast cell pellets or (FIG. 1B) cream yeast samples of wild-type (M10474) or recombinant yeast host cells. In FIG. 1A, results are shown as the maltogenic amylase activity (provided as MANU/mL) in function of type of yeast tested (from left to right, M10474, T2986, T2987, T2988, T2989, T2990, T2991, T2944; strains are described in Table 1). In FIG. 1B, results are shown as the maltogenic amylase activity (provided as MANU/mL) in function of type and lot of yeast tested (from left to right, M10474, M13819, M13822; strains are described in Table 1).

FIGS. 2A to 2C provide crumb hardness (as measured in grams, left axis, shown in the bars) and bread volume (as measured in centimeter cubes, right axis, as shown in the -X- labelled line) in function of bread made with (from left to right) the wild-type strain (M10474) with no enzyme supplementation, wild-type strain supplemented with 45 ppm Novamyl®, wild-type strain supplemented with 90 ppm Novamyl®, wild-type strain supplemented with 180 ppm Novamyl®, M13822 strain (lot A), M13822 strain (lot B), M13819 (lot A) and M13819 (lot B) (strains are described in Table 1). Results are shown 5 (FIG. 2A), 8 (FIG. 2B) and 11 (FIG. 2C) days after baking.

FIGS. 3A to 3C provide percent resilience in function of bread made with (from left to right) the wild-type strain (M10474) with no enzyme supplementation, wild-type strain supplemented with 45 ppm Novamyl®, wild-type strain supplemented with 90 ppm Novamyl®, wild-type strain supplemented with 180 ppm Novamyl®, M13822 strain (lot A), M13822 strain (lot B), M13819 (lot A) and M13819 (lot B). Results are shown 5 (FIG. 3A), 8 (FIG. 3B) and 11 (FIG. 3C) days after baking.

FIG. 4 provides the glucoamylase enzyme activity measured in pellets (“bound”, light gray) and supernatant (“free”, dark gray) of cultured recombinant yeast host cells expressing an heterologous glucoamylase in the absence (strain M8498) and in the presence (strain M14244) of a Sed1 tether. Results are shown as percentage of glucoamylase activity in function of strain used.

FIG. 5 provides alpha-amylase enzyme activity measured in pellets (“bound”, light gray) and supernatant (“free”, dark gray) of cultured recombinant yeast host cells expressing an heterologous alpha-amylase in the presence of a Sed1 tether and a linker (strain M14253), in the presence of a Sed1 tether but no linker (M14254) and in the absence of a Sed1 tether (strain M10074). Results are shown as percentage of alpha-amylase activity in function of strain used.

FIG. 6 provides wheat starch activity of various strains expressing a maltogenic amylase. Results are shown as wheat starch MANU per mL (measured at OD 600 nm) for the whole culture (left bars), supernatant (middle bars) and washed pellet (right bars) of the M10474, M13822, M13819, M13879 and T3892 strains (strains are described in Table 1). Data for “M” strains are the average of duplicate cultures. Data for T3892 include the average activity across cultures of eight transformations isolates and the activity of the top performing isolate (□=top isolate, whole culture; Δ=top isolate, supernatant; ○=top isolate, washed cell pellet). Graphics below indicate the predicted enzyme localization phenotype of each engineering strategy.

FIGS. 7A and 7B provide the phytase activity in culture supernatant (gray bars) or associated with cells (diagonally hatched bars in FIG. 7A or □ in FIG. 7B) for strains expressing free or tethered Citrobacter braakii phytase. Supernatant was incubated with 5 mM sodium phytate solution pH 5.5 for 30 minutes and cells were incubated in the same solution for 2 hours. (FIG. 7A) Absorbance at 700 nm was compared to a standard curve of known phosphate concentrations to express activity in FTUs. The absorbance was measured in the supernatant (grey bars) and the cells (diagonal bars) in different strains (M12548, T2633, T2634, T2635, T2636, T2637 and T2638). (FIG. 7B) FTU were compared between the different strains. The left vertical axis shows supernatant activity and the FTU for each strains is provided as the grey bars. The right axis shows cell-associated FTU activity and is provided as □ for each strains (M12548, T2633, T2634, T2635, T2636, T2637 and T2638). The values for the parent strain and the Pst1 tether cell associated activity were outside the range of the standard curve and therefore below the detection limit.

FIG. 8 provides the phytase activity in culture supernatant (grey bars) or associated with cells (diagonally hatched bars) for strains expressing Escherichia coli phytase fused with either an N- or C-terminal tether. Supernatant was incubated with 5 mM sodium phytate solution pH 5.5 for 30 minutes and cells were incubated in the same solution for 2 hours. Results are shown as the optical density at 700 nm in function of each strain (M11312, T2705 and T2706).

FIG. 9 provides the phytase activity in culture supernatant (grey bars) or associated with cells (diagonally hatched bars) for strains expressing E. coli phytase fused with either an N-terminal tether with or without overexpression of AGA1 compared to E. coli phytase fused with a C-terminal Sed1 tether. Supernatant was incubated with 5 mM sodium phytate solution pH 5.5 for 30 minutes and cells were incubated in the same solution for 2 hours. Results are shown as the optical density at 700 nm in function of each strain (M12550, M12795, M12983 and T2816).

FIG. 10 provides the wheat starch activity of strains expressing maltogenic amylase. Results are provided as the ratio of absorbance at 450 nm/optical density at 600 nm for the whole culture (left bars), the supernatant (middle bars) and washed cells (left bars) for the different strains (M10474, M13819, M13822, M14851, T4328, T4329, T4330, M12962, T4336, T4337 and T4338). Data for “M” strains are the average of duplicate cultures. Data for “T” strains include the average activity across cultures of seven transformations isolates. Expression type 1 refers to the presence of an invertase signal peptide and a Spi1 tether to generate a tethered enzyme. Expression type 2 refers to the presence of an invertase signal peptide and the absence of a tether to generate a secreted enzyme. Expression type 3 refers to the absence of a signal peptide and the absence of a tether to generate an intracellular enzyme.

FIG. 11A to 11C provide the crumb hardness (as measured in grams, left axis, results shown in the bars) of breads made with different dough conditioners at days 3 (FIG. 11A), 7 (FIG. 11B) and 11 (FIG. 11C) after baking. Controls were made with (labelled “Novamyl”) or without (labelled “Control”) externally added the Novamyl® maltogenic amylase product as indicated below the histogram. The control breads were compared to breads made with M13979 spray-dried homogenate (identified as “homo+spray” in the figures) or cream dosed to a specific Phadebas enzyme activity as indicated below the histogram. All breads used wild-type yeast for gassing power.

FIGS. 12A to 12F provide the crumb hardness (FIG. 12A-C, as measured in grams) and resilience (FIG. 12D-F, as measured in percentage) of breads made with different dough conditioners at days 4 (FIG. 12A and FIG. 12D), 7 (FIG. 12B and FIG. 12E) and 10 (FIG. 12C and FIG. 12F) after baking. Controls were made with (labelled “Novamyl”) or without (labelled “Control”) externally added the Novamyl® maltogenic amylase product as indicated below the histogram. The control breads were compared to breads made with M15532 yeast cream that was homogenized to release the intracellular and dosed to a specific Phadebas enzyme assay activity as indicated below the histogram. All breads used wild-type yeast for gassing power.

FIG. 13 shows the alpha-amylase activity associated with the cells of yeast strains expressing various chimeric proteins comprising a thermo-tolerant alpha-amylase derived from Pyrococcus furiosus (SEQ ID NO: 71) in combination with different tethering moieties derived from the SPI1 protein or associated truncations (M15774, M15771, M15777, M15772 and M15222) compared to a control strain (M2390). Results are shown as the absorbance at 540 nm in function of the yeast strain used.

FIG. 14 shows the alpha-amylase activity associated with cells of yeast strains expressing various chimeric proteins comprising an alpha-amylases derived from Thermococcus hydrothermalis (SEQ ID NO: 72) in combination with different tethering moieties derived from the CCW12 protein or associated truncations (M15773, M15776, M16251 and M15215) compared to a control strain (M2390). Results are shown as the absorbance at 540 nm in function of the yeast strain used.

FIG. 15 shows the alpha-amylase activity associated with the cells of yeast strains expressing various chimeric proteins comprising an alpha-amylase derived from T. hydrothermalis (SEQ ID NO: 72) in combination with a tethering moiety derived from the CCW12 protein and different linkers (M15785, M15786, M15782, M16252, M16221 and M16222) compared to a control strain (M2390). Results are shown as the absorbance at 540 nm in function of the yeast strain.

FIG. 16 shows the alpha-amylase activity associated with the cells of yeast strains expressing various chimeric proteins comprising an alpha-amylase derived from P. furiosus (SEQ ID NO: 71), a tethering moiety derived from the SPI1 protein and different linkers (M15784, M15778, M15779, M15787, M15780, M15788 and M15783) compared to a control strain (M2390). Results are shown as the absorbance at 540 nm in function of the yeast strain.

FIG. 17 shows the glucose oxidase (GO) activity associated with the whole culture (grey bars), washed cells (diagonal hatch bars) or the supernatant of disrupted washed cells (white bars) of yeast strains expressing a glucose oxidase derived from Aspergillus niger, expressed in a secreted form (M16780) or intracellularly (M16273) compared to a negative control strain (M10474) and a positive control amount of a commercially available purified glucose oxidase (positive control, Gluzyme Mono®). Results are shown as absorbance at 510 nm in function of the yeast strain/control used.

FIG. 18 shows the glucose oxidase (GO) activity associated with the whole culture (grey bars), washed cells (diagonal hatch bars) of yeast strains expressing a glucose oxidase derived from Aspergillus niger, expressed in a secreted form (M16780) or intracellularly (M16273). Results are shown as absorbance at 510 nm (corrected to remove the absorbance associated with control strain M10474) in function of the yeast strain used.

FIG. 19 shows the fungal amylase (FA) activity associated with the whole culture (grey bars), washed cells (diagonal bars) or the supernatant of disrupted washed cells (white bars) of yeast strains expressing a fungal amylase derived from Aspergillus oryzae expressed in a secreted form with a different signal peptides (S. cerevisiae invertase for M16772, A. oryzae native alpha-amylase signal peptide for M16540) compared to a negative control strain (M10474) and a positive control amount of a commercially available purified fungal alpha-amylase (positive control, Fungamyl®). Results are shown as absorbance at 540 nm in function of the yeast strain/control used.

FIG. 20 shows the fungal amylase (FA) activity associated with the whole culture (grey bars), washed cells (diagonal hatch bars) or the supernatant of disrupted washed cells (white bars) of yeast strains expressing a fungal amylase derived from Aspergillus oryzae expressed in a secreted form with a different signal peptides (S. cerevisiae invertase for M16772, A. oryzae native alpha-amylase signal peptide for M16540). Results are shown as absorbance at 540 nm (corrected to remove the absorbance associated with control strain M10474) in function of the yeast strain used.

FIG. 21 shows the evaluation of cell-associated glucose oxidase activity from a cell pellet of strain M16780 using a bake test. Results are shown for control loaves (prepared in the absence of an additive), for loaves prepared with 10 ppm or 20 ppm Gluzyme Mono® or for loaves prepared with a dosed the cell pellet of strain M16780.

### DETAILED DESCRIPTION

The present disclosure provides recombinant yeast host cells expressing a cell-associated heterologous food and/or feed enzyme during their propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (usually under anaerobic conditions) to maximize the production of one or more desired metabolite and/or make a fermented food or fee product. The recombinant yeast host cells of the present disclosure are advantageous because they provide a lower cost source of enzyme activity than the purified products that are traditionally used. Such recombinant yeast host cells can be advantageously used in various food and/or feed products, such as, for example, baked products, even though the proofing time and conditions do not provide an opportunity for the yeast to produce the enzymes in situ. Such recombinant yeast host cells can also be used in other baked products, fermented foods, non-fermented foods and animal feed. The recombinant yeast host cells can advantageously be easily measured, dosed and formulated.

### Recombinant Yeast Host Cells

The recombinant yeast host cells of the present disclosure are intended to be used for making products for human (food) and/or animal (feed) consumption. As used in the context of the present disclosure, the expression “food and/or feed enzyme” refers to a protein having enzymatic activity and capable of being used in a process for making a food product or a feed product. In some embodiments, the “food and/or feed enzyme” refers to enzymes having applications in transforming starchy (e.g., starch-containing biomass). Food and feed enzymes include, without limitation, baking enzymes, brewing enzymes, distilling enzymes, winemaking enzymes, juice enzymes, starch processing enzymes and feed enzymes. The recombinant yeast host cells of the present disclosure can optionally be used in a fermentation process. In an embodiment, the fermentation process can be a relatively long one and the recombinant yeast host cells can be used, for example, in making distilling products, wine and beer. In another embodiment, the fermentation process can be a relatively short one and the recombinant yeast host cells can be used, for example, in making yeast-leavened bakery products. The recombinant yeast host cells of the present disclosure can also be used in a process which does not include a fermentation step. For example, the recombinant yeast host cell can be used for making food and beverages (e.g., non-yeast-leavened (chemically-leavened) bakery products, dairy products, yeast extracts, juices, fat and oils as well as starch), or feed.

In an embodiment, the recombinant yeast host cells of the present disclosure do express at least one food and/or feed enzyme prior to the introduction of heterologous nucleic acid molecules of the present disclosure and are genetically modified to express a further (a different or the same) cell-associated enzyme. In another embodiment, the recombinant yeast host cells of the present disclosure cannot be used in consolidated bioprocessing for making, for example, biofuels such as bioethanol.

The recombinant yeast host cells of the present disclosure can be provided in an active form (e.g., liquid (such as, for example, a cream yeast), compressed, or fluid-bed dried yeast), in a semi-active form (e.g., liquid, compressed, or fluid-bed dried), in an inactive form (e.g., drum- or spray-dried) as well as a mixture therefore. For example, the recombinant yeast host cells can be a combination of active and semi-active or inactive forms to provide the ratio and dose of the enzyme required for making the food or feed product.

The present disclosure concerns recombinant yeast host cells that have been genetically engineered. The genetic modification(s) is(are) aimed at increasing the expression of a specific targeted gene (which is considered heterologous to the yeast host cell) and can be made in one or multiple (e.g., 1, 2, 3, 4, 5, 6, 7, 8 or more) genetic locations. In the context of the present disclosure, when recombinant yeast cell is qualified as being “genetically engineered”, it is understood to mean that it has been manipulated to add at least one or more heterologous or exogenous nucleic acid residue. In some embodiments, the one or more nucleic acid residues that are added can be derived from an heterologous cell or the recombinant host cell itself. In the latter scenario, the nucleic acid residue(s) is (are) added at one or more genomic location which is different than the native genomic location. The genetic manipulations did not occur in nature and are the results of in vitro manipulations of the yeast.

When expressed in a recombinant yeast host cells, the heterologous enzymes described herein are encoded on one or more heterologous nucleic acid molecules. The term “heterologous” when used in reference to a nucleic acid molecule (such as a promoter, a terminator or a coding sequence) or a protein (such as an enzyme) refers to a nucleic acid molecule or a protein that is not natively found in the recombinant host cell. “Heterologous” also includes a native coding region/promoter/terminator, or portion thereof, that was removed from the source organism and subsequently reintroduced into the source organism in a form that is different from the corresponding native gene, e.g., not in its natural location in the organism's genome. The heterologous nucleic acid molecule is purposively introduced into the recombinant host cell. For example, a heterologous element could be derived from a different strain of host cell, or from an organism of a different taxonomic group (e.g., different kingdom, phylum, class, order, family genus, or species, or any subgroup within one of these classifications).

The heterologous nucleic acid molecule present in the recombinant host cell can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies (e.g., 2, 3, 4, 5, 6, 7, 8 or even more copies) in the yeast host cell's genome. Alternatively, the heterologous nucleic acid molecule can be independently replicating from the yeast's genome. In such embodiment, the nucleic acid molecule can be stable and self-replicating.

In the context of the present disclosure, the recombinant host cell is a yeast and in some embodiments the yeast can be used in the production of food and/or feed. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, C. utilis, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiment, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the host cell can be an oleaginous microalgae host cell (e.g., for example, from the genus Thraustochytrium or Schizochytrium). In an embodiment, the recombinant yeast host cell is from the genus Saccharomyces and, in some embodiments, from the species Saccharomyces cerevisiae.

The recombinant yeast host cells of the present disclosure include an heterologous nucleic acid molecule intended to allow the expression of (e.g., encode) one or more heterologous food and/or feed enzymes. In an embodiment, the heterologous enzyme is a food enzyme which can be, without limitation, alpha-acetolactate decarboxylase, aminopeptidase, amylase, maltogenic alpha-amylase, asparaginase, bromelain, carboxypeptidase, catalase, cellulase, chymosin (including chymosin A and B), cyprosin, ficin, glucoamylase (also known as amyloglucosidase or maltase), glucanase, glucose oxidase, glucose isomerase, hemicellulase, hexose oxidase, inulinase, invertase, lactase, lipase, lipoxidase, lysozyme, mannanase, milk coagulating enzyme, pancreatin, papain, pectinase, pentosanase, pepsin, phospholipase, peroxidase, protease, pullulanase, rennet (including bovine rennet), transglutaminase, trypsin, urease and/or xylanase. In an embodiment, the heterologous food and/or feed enzyme is a baking enzyme. As used in the context of the present disclosure, the expression “baking enzyme” refers to a protein having enzymatic activity and capable of being used in a process for making a baked product. In an embodiment, the heterologous nucleic acid molecule of the yeast host cells of the present encodes at least one heterologous baking enzyme. Baking enzymes, include, without limitation, amylolytic enzymes (including, for example, maltogenic alpha-amylases, glucoamylases, alpha-amylases and fungal amylases), cellulases/hemicellulases, oxidases (including, for example, glucose oxidases), asparaginases, and lipases. In another embodiment, the heterologous enzyme is a feed enzyme which can be, without limitation, a phytase, β-glucanase, xylanase, alpha-galactosidase, protease, amylase, lipase, mannanase, cellulase and/or hemicellulasespectinase.

As used herein, the expression “amylolytic enzyme” refers to a class of enzymes capable of hydrolyzing starch or hydrolyzed starch. In baking applications, amylolytic enzymes can participate in releasing of fermentable sugars, increasing bread volume, decreasing fermentation time, reducing staling and/or improving flavor. Amylolytic enzymes include, but are not limited to alpha-amylases (EC 3.2.1.1, sometimes referred to fungal alpha-amylases as well as bacterial alpha-amylases, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68), and amylomaltase (EC 2.4.1.25). Fungal alpha-amylases can be used, for example, in the production of baked products (for example, yeast-leavened, chemically-leavened or unleavened products), juices and fermented beverages (like beers). Bacterial α-amylases can be used, for example, in the production of baked products (for example, yeast-leavened, chemically-leavened or unleavened products), fermented beverages (including beers, distilled beverages and the like) as well as in the processing of starch. Maltogenic alpha-amylases can be used, for example, in the production of baked products (for example, yeast-leavened, chemically-leavened or unleavened products). In an embodiment, the one or more amylolytic enzymes can be an α-amylase from Aspergillus oryzae (and have, for example, the amino acid sequence of SEQ ID NO: 2 or 105, a variant thereof or a fragment thereof), a maltogenic α-amylase from Geobacillus stearothermophilus (and have, for example, the amino acid sequence of SEQ ID NO: 1, 51, 65 or 108, a variant thereof or a fragment thereof), a glucoamylase from Saccharomycopsis fibuligera (and have, for example, the amino acid sequence of SEQ ID NO: 3, a variant thereof or a fragment thereof), a glucan 1,4-alpha-maltotetraohydrolase from Pseudomonas saccharophila (and have, for example, the amino acid sequence of SEQ ID NO: 4, a variant thereof or a fragment thereof), a pullulanase from Bacillus naganoensis (and have, for example, the amino acid sequence of SEQ ID NO: 5, a variant thereof or a fragment thereof), a pullulanase from Bacillus acidopullulyticus (and have, for example, the amino acid sequence of SEQ ID NO: 6, a variant thereof or a fragment thereof), an iso-amylase from Pseudomonas amyloderamosa (and have, for example, the amino acid sequence of SEQ ID NO: 7, a variant thereof or a fragment thereof), and/or amylomaltase from Thermus thermophilus (and have, for example, the amino acid sequence of SEQ ID NO: 8, a variant thereof or a fragment thereof).

As used herein, the expression “cellulase/hemi-cellulase” refers to a class of enzymes capable of hydrolyzing cellulose, hemi-cellulose, or pentosans. In baking applications, cellulases and hemi-cellulases can participate in establishing the gluten network, providing a soluble dietary fiber, modulating dough viscosity and/or modulating dough rheology. Cellulases/hemi-cellulases include, but are not limited to a cellulase (E.C. 3.2.1.4) and an endoB(1,4)D-xylanase (E.C. 3.2.1.8). In an embodiment, the one or more cellulase/hemi-cellulase can be a cellulase from Penicillium funiculosum (and have, for example, the amino acid sequence of SEQ ID NO: 42, a variant thereof or a fragment thereof) and/or an endoB(1,4)D-xylanase from Rasamsonia emersonii (and have, for example, the amino acid sequence of SEQ ID NO: 43, a variant thereof or a fragment thereof).

As used herein, the expression “oxidase” refers to a class of enzymes capable of catalyzing an oxidation-reduction reaction. The oxidase can be an oxidoredductase such as an hexose oxidase (including a glucose oxidase). Oxidases can be used in the production of baked products (such as, for examples, yeast-leavened products including bread). In some embodiments, oxidases (such as glucose oxidases) can improve dough machinability. In baking applications, oxidases can participate in controlling of Maillard reactions and/or establishing crumb structure. In an embodiment, the one or more oxidases can be a glucose oxidase from Aspergillus niger (and have, for example, the amino acid sequence of SEQ ID NO: 44 or 103, a variant thereof or a fragment thereof).

As used herein, the expression “asparaginase” refers to a class of enzymes capable of catalyzing the conversion of asparagine into aspartic acid and ammonium. Asparaginase can be used in the production of snacks, cereals (including breakfast cereals) as well as baked products (for example, yeast-leavened (including bread), chemically-leavened or unleavened products).

As used herein, the expression “lipase” refers to a class of enzymes capable of hydrolyzing lipids. In baking applications, lipases can participate in increasing bread volume, increasing dough stability, providing anti-staling and/or facilitating emulsifier formations. Lipases can be used, for example, in the production of baked products (such as yeast-leavened (including bread) and chemically-leavened products). In an embodiment, the one or more lipase can be a triacylglycerol lipase from Thermomyces lanuginosis (and have, for example, the amino acid sequence of SEQ ID NO: 45, a variant thereof or a fragment thereof), a phospholipase A2 from Sus scrofa (and have, for example, the amino acid sequence of SEQ ID NO: 46, a variant thereof or a fragment thereof), a phospholipase A2 from Streptomyces vialaceoruber (and have, for example, the amino acid sequence of SEQ ID NO: 47, a variant thereof or a fragment thereof) and/or a phospholipase A2 from Aspergillus oryzea (and have, for example, the amino acid sequence of SEQ ID NO: 48, a variant thereof or a fragment thereof).

In an embodiment, the recombinant yeast host cell of the present disclosure includes (and in an embodiment expresses) a nucleic acid molecule coding for a maltogenic amylase. As used in the present disclosure, the term “maltogenic amylase” refers to a polypeptide capable of hydrolyzing starch or hydrolyzed starch into maltose. Maltogenic amylases include, but are not limited to fungal alpha-amylases (derived, for example, from Aspergillus sp. (e.g., A. Niger, A. kawachi, and A. oryzae); Trichoderma sp. (e.g., T. reesie), Rhisopus sp., Mucor sp., and Penicillium sp.), acid stable fungal amylase (derive, for example, from Aspergillus niger), beta-amylases (derived, for example, from plant (wheat, barley, rye, shorgum, soy, sweet potato, rice) and microorganisms (Bacillus cereus, Bacillus polymixa, Bacillus megaterium, Arabidopsis thaliana), maltogenic amylases (E.C.3.2.1.133) (derived, for example, from microorganisms such as Bacillus subtilis, Geobacillus stearothermophilus, Bacillus thermoalkalophilus, Lactobacillus gasseri, Thermus sp.). In a specific embodiment, the recombinant yeast host cells of the present disclosure include an heterologous nucleic acid molecule coding for the heterologous maltogenic amylase derived from Geobacillus stearothermophilus and having, for example, the amino acid sequence of SEQ ID NO: 1, 51, 65 or 108, a variant thereof or a fragment thereof.

As used herein, the expression “phosphatase” refers to a food/feed enzyme capable, in the presence of water, of catalyzing the cleavage of a phosphoric acid monoester into a phosphate ion and an alcohol. An embodiment of a phosphatase is a phytase, a protein having enzymatic activity and capable of catalyzing the hydrolysis of phytic acid (myo-inositol hexakisphosphate) into inorganic phosphorus. There are four distinct classes of phytase: histidine acid phosphatases (HAPS), beta-propeller phytases, purple acid phosphatases and protein tyrosine phosphatase-like phytases (PTP-like phytases). Phytic acid has six phosphate groups that may be released by phytases at different rates and in different order. Phytases hydrolyze phosphates from phytic acid in a stepwise manner, yielding products that again become substrates for further hydrolysis. Phytases have been grouped based on the first phosphate position of phytic acid that is hydrolyzed: are 3-phytase (EC 3.1.3.8), 4-phytase (EC 3.1.3.26) and 5-phytase (EC 3.1.3.72). In an embodiment, the phytase is derived from a bacterial species, such as, for example, a Citrobacter sp. or an Escherichia sp. In a specific embodiment, the heterologous phytase is derived from a Citrobacter sp., such as for example Citrobacter braakii and can have, for example, the amino acid sequence of SEQ ID NO: 66, a variant thereof or a fragment thereof. In another embodiment, the heterologous phytase is derived from an Escherichia sp., such as, for example, Escherichia coli and can have, for example, the amino acid sequence of SEQ ID NO: 67, a variant thereof or a fragment thereof.

The heterologous food and/or feed enzyme can be a variant of a known/native food and/or feed enzyme. For example, in embodiments in which the heterologous food and/or feed enzyme is an heterologous baking enzyme, the heterologous baking enzyme can be a variant of a known/native baking enzyme, for example a variant of the heterologous baking enzyme having the amino acid sequence of SEQ ID NO: 1, 2, 3, 4, 5, 6, 7, 8, 42, 43, 44, 45, 46, 47, 48, 51, 65, 66, 67, 103, 105 or 108. A variant comprises at least one amino acid difference when compared to the amino acid sequence of the native food and/or feed enzyme. As used herein, a variant refers to alterations in the amino acid sequence that do not adversely affect the biological functions of the food and/or feed enzyme. A substitution, insertion or deletion is said to adversely affect the protein when the altered sequence prevents or disrupts a biological function associated with the food and/or feed enzyme. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the food and/or feed enzyme. The food and/or feed enzyme variants have at least 50%, 55%, 60%, 65%, 70%, 75%, 80%, 85%, 90%, 95%, 96%, 97%, 98% or 99% identity to the food and/or feed enzymes described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLB 1, GAP PENALTY=3, WINDOW=5 and DIAGONALS SAVED=5.

The variant heterologous food and/or feed enzymes described herein (including the food and/or feed enzymes described herein) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the food and/or feed enzyme can be a conservative variant or an allelic variant.

The heterologous food and/or feed enzyme can be a fragment of a known/native food and/or feed enzymes. In embodiments in which the heterologous food and/or feed enzyme is an heterologous baking enzyme, the heterologous baking enzyme can be a fragment of a known/native baking enzyme or fragment of a variant of a known/native baking enzyme (such as, for example, a fragment of the baking enzyme having the amino acid sequence of SEQ ID NO: 1, 2, 3, 4, 5, 6, 7, 8, 42, 43, 44, 45, 46, 47, 48, 51, 65, 66, 67, 103, 105 or 108 or a variant thereof). In an embodiment, a fragment corresponds to the known/native food and/or feed enzyme to which the signal peptide sequence has been removed. Food and/or feed enzyme “fragments” (including baking enzyme “fragments”) have at least at least 100, 200, 300, 400, 500, 600, 700 or more consecutive amino acids of the food and/or feed enzyme. A fragment comprises at least one less amino acid residue when compared to the amino acid sequence of the known/native food and/or feed enzyme and still possess the enzymatic activity of the full-length food and/or feed enzyme. In an embodiment, the fragment corresponds to the amino acid sequence of the enzyme lacking the signal peptide. In some embodiments, fragments of the food and/or feed enzymes can be employed for producing the corresponding full-length food and/or feed enzymes by peptide synthesis. Therefore, the fragments can be employed as intermediates for producing the full-length proteins.

In the recombinant yeast host cell of the present disclosure, the heterologous food and/or feed enzyme (including the baking enzyme) is “cell-associated” to the recombinant yeast host cell because it is designed to be expressed and remain physically associated with the recombinant yeast host cells. In an embodiment, the food and/or feed enzyme can be expressed inside the recombinant yeast host cell (intracellularly). In such embodiment, the heterologous food and/or feed enzyme does not need to be associated to the recombinant yeast host cell's wall. When the food and/or feed enzyme is intended to be expressed intracellularly, its signal peptide sequence, if present in the native sequence, can be deleted to allow intracellular expression.

In another embodiment, the heterologous food and/feed enzyme can be secreted, but if it is, it must remain physically associated with the recombinant yeast host cell. In an embodiment, at least one portion (usually at least one terminus) of the heterologous food and/or feed enzyme is bound, covalently, non-covalently and/or electrostatically for example, to the cell wall (and in some embodiments to the cytoplasmic membrane). For example, the heterologous food and/or feed enzyme can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous food and/or feed enzyme may not be directly bound to the cell membrane or cell wall (e.g., such as when binding occurs via a tethering moiety), the protein is nonetheless considered a “cell-associated” heterologous food and/or feed enzyme according to the present disclosure.

In some embodiments, the heterologous food and/or feed enzyme can be expressed to be located at and associated to the cell wall of the recombinant yeast host cell. In some embodiments, the heterologous food and/or feed enzyme is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The heterologous food and/or enzyme can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous food and/or feed enzyme to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous food and/or feed enzyme, a domain capable of interacting with a cytoplasmic membrane protein on the heterologous food and/or feed enzyme, a post-translational modification made to the heterologous enzyme food and/or feed enzyme (e.g., lipidation), etc.

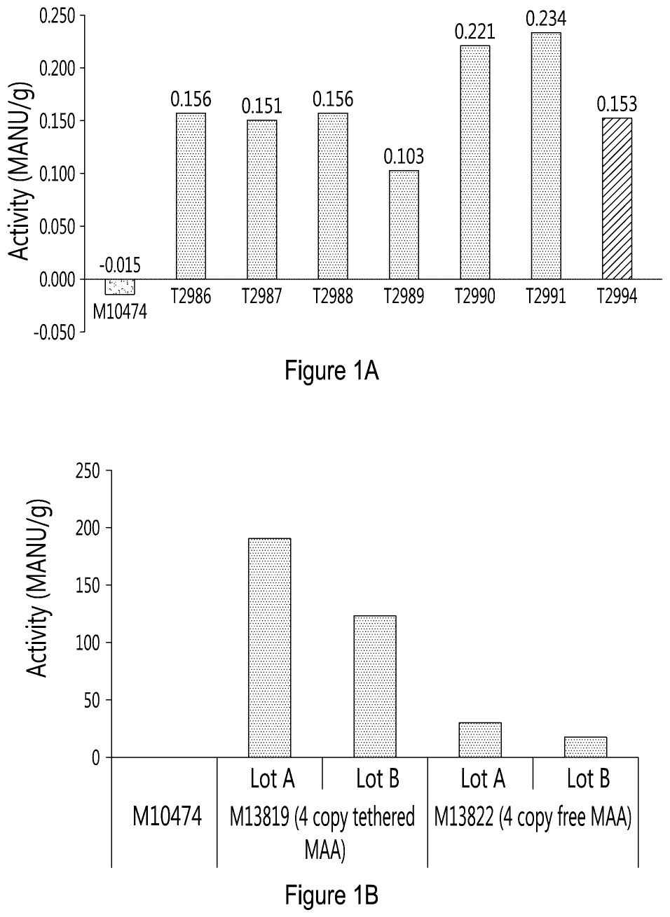

Some heterologous food and/or feed enzymes (including baking enzymes) have the intrinsic ability to locate at and associate to the cell wall of a recombinant yeast host cell (e.g., being cell-associated). One example of a food and/or feed enzyme having the intrinsic ability of being cell-associated is shown in FIG. 1A moiety (e.g., strain T2994 column in FIG. 1A). In this figure, results are presented for the maltogenic alpha-amylase of Geobacillus stearothermophilus expressed in S. cerevisiae in the absence of a tethering moiety and clearly show that this enzyme is intrinsically “cell-associated” and exhibits enzymatic activity (e.g., maltogenic alpha-amylase activity).

However, in some circumstances, it may be warranted to increase or provide cell association to some food and/or feed enzymes because they exhibit insufficient intrinsic cell association or simply lack intrinsic cell association. In such embodiment, it is possible to provide the heterologous food and/or feed enzyme as a chimeric construct by combining it with a tethering amino acid moiety which will provide or increase attachment to the cell wall of the recombinant yeast host cell. In such embodiment, the chimeric food and/or feed enzyme will be considered “tethered”. It is preferred that the amino acid tethering moiety of the chimeric protein be neutral with respect to the biological (enzymatic) activity of the heterologous food and/or feed enzyme, e.g., does not interfere with the biological (enzymatic) activity of the heterologous food and/or feed enzyme. In some embodiments, the association of the amino acid tethering moiety with the heterologous food and/or feed enzyme can increase the biological (enzymatic) activity of the heterologous food and/or feed enzyme (when compared to the non-tethered, non-chimeric form).

In an embodiment, a tethering moiety can be used to be expressed with the heterologous food and/or feed enzyme to locate the enzyme to the wall of the recombinant yeast host cell. Various tethering amino acid moieties are known art and can be used in the chimeric proteins of the present disclosure.

The tethering moiety can be a transmembrane domain found on another protein and allow the chimeric protein to have a transmembrane domain. In such embodiment, the tethering moiety can be derived from the FLO1 protein (having, for example, the amino acid sequence of SEQ ID NO: 10, a variant thereof or a fragment thereof or being encoded by the nucleic acid sequence of SEQ ID NO: 9).

In still another example, the amino acid tethering moiety can be modified post-translation to include a glycosylphosphatidylinositol (GPI) anchor and allow the chimeric protein to have a GPI anchor. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein (having, for example, the amino acid sequence of SEQ ID NO: 12, a variant thereof or a fragment thereof or being encoded by the nucleic acid sequence of SEQ ID NO: 11), a TIR1 protein (having, for example, the amino acid sequence of SEQ ID NO: 14, a variant thereof or a fragment thereof or being encoded by the nucleic acid sequence of SEQ ID NO: 13), a CWP2 protein (having, for example, the amino acid sequence of SEQ ID NO: 16, a variant thereof or a fragment thereof or being encoded by the nucleic acid sequence of SEQ ID NO: 15), a CCW12 protein (having, for example, the amino acid sequence of SEQ ID NO: 18 or 84, a variant thereof or a fragment thereof or being encoded by the nucleic acid sequence of SEQ ID NO: 17), a SPI1 protein (having, for example, the amino acid sequence of SEQ ID NO: 20 or 74, a variant thereof or a fragment thereof or being encoded by the nucleic acid sequence of SEQ ID NO: 19), a PST1 protein (having, for example, the amino acid sequence of SEQ ID NO: 22, a variant thereof or a fragment thereof or being encoded by the nucleic acid sequence of SEQ ID NO: 21) or a combination of a AGA1 and a AGA2 protein (having, for example, the amino acid sequence of SEQ ID NO: 24, a variant thereof or a fragment thereof or being encoded by the nucleic acid sequence of SEQ ID NO: 23 or having, for example, the amino acid sequence of SEQ ID NO: 26, a variant thereof or a fragment thereof or being encoded by the nucleic acid sequence of SEQ ID NO: 25). In an embodiment, the tethering moiety provides a GPI anchor and, in still a further embodiment, the tethering moiety is derived from the SPI1 protein (having, for example, the amino acid sequence of SEQ ID NO: 20 or 74, a variant thereof or a fragment thereof or being encoded by the nucleic acid sequence of SEQ ID NO: 19) or the CCW12 protein (having, for example, the amino acid sequence of SEQ ID NO: 18 or 84, a variant thereof or a fragment thereof or being encoded by the nucleic acid sequence of SEQ ID NO: 17).

In an embodiment, the tethering moiety is a fragment of the SPI1 protein that retained its ability to localize to the cell's membrane. The fragment of the SPI1 protein comprises less than 129 amino acid consecutive residues of the amino acid sequence of SEQ ID NO: 74. For example, the tethering moiety fragment from the SPI1 protein can comprise at least 10, 20, 21, 30, 40, 50, 51, 60, 70, 80, 81, 90, 100, 110, 111 or 120 consecutive amino acid residues from the amino acid sequence of SEQ ID NO: 74. In yet another embodiment, the tethering moiety fragment from the SPI1 protein can comprise or consist essentially of the amino acid sequence set forth in any one of SEQ ID NOs: 76, 78, 80 or 82.

In another embodiment, the tethering moiety is a fragment of a CCW12 protein that retained its ability to localize to the cell's membrane. The fragment of the CCW12 protein comprises less than 112 amino acid consecutive residues of the amino acid sequence of SEQ ID NO: 84. For example, the tethering moiety fragment from the CCW12 protein can comprise at least 10, 20, 24, 30, 40, 49, 50, 60, 70, 74, 80, 90, 99, 100 or 110 consecutive amino acid residues from the amino acid sequence of SEQ ID NO: 84. In yet another embodiment, the tethering moiety fragment from the CCW12 protein can comprise or consist essentially of the amino acid sequence set forth in any one of SEQ ID NOs: 86, 88, 90 or 92.

The tethering amino acid moiety can be a variant of a known/native tethering amino acid moiety, for example a variant of the tethering amino acid moiety having the amino acid sequence of SEQ ID NOs: 10, 12, 14, 16, 18, 20, 22, 24, 26, 74, 76, 78, 80, 84, 82, 86, 88, 90 or 92. A variant comprises at least one amino acid difference when compared to the amino acid sequence of the native tethering amino acid moiety. As used herein, a variant refers to alterations in the amino acid sequence that do not adversely affect the biological functions of the tethering amino acid moiety (e.g., the location on the external face and the anchorage of the heterologous food and/or feed enzyme in the cytoplasmic membrane). A substitution, insertion or deletion is said to adversely affect the protein when the altered sequence prevents or disrupts a biological function associated with the tethering amino acid moiety (e.g., the location on the external face and the anchorage of the heterologous food and/or feed enzyme in the cytoplasmic membrane). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the tethering amino acid moiety. The tethering amino acid moiety variants have at least 50%, 55%, 60%, 65%, 70%, 75%, 80%, 85%, 90%, 95%, 96%, 97%, 98% or 99% identity to the tethering amino acid moieties described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLB 1, GAP PENALTY=3, WINDOW=5 and DIAGONALS SAVED=5.

The variant tethering amino acid moieties described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the tethering amino acid moiety can be a conservative variant or an allelic variant.

The tethering amino acid moiety can be a fragment of a known/native tethering amino acid moiety or fragment of a variant of a known/native tethering amino acid moiety (such as, for example, a fragment of the tethering amino acid moiety having the amino acid sequence of SEQ ID NO: 10, 12, 14, 16, 18, 20, 22, 24, 26, 74, 76, 78, 80, 82, 84, 86, 88, 90 or 92 or a variant thereof). Tethering amino acid moiety “fragments” have at least at least 10, 20, 30, 40, 50, 60, 70, 80, 90, 100 or more consecutive amino acids of the tethering amino acid moiety. A fragment comprises at least one less amino acid residue when compared to the amino acid sequence of the known/native tethering amino acid moiety and still possess the biological activity of the full-length tethering amino acid moiety (e.g., the location to the cell wall).

In embodiments in which an amino acid tethering moiety is desirable, the heterologous food and/or feed enzyme can be provided as a chimeric protein expressed by the recombinant yeast host cell and having one of the following formulae (provided from the amino (NH₂) to the carboxyl (COOH) orientation):

$$F F E - L - TT or \qquad ( I )$$

$$TT - L - FFE \qquad ( II )$$

In both of these formulae, the residue “FFE” refers to the heterologous food and/or feed enzyme moiety, the residue “L” refers to the presence of an optional linker while the residue “TT” refers to an amino acid tethering moiety. In the chimeric proteins of formula (I), the amino terminus of the amino acid tether is located (directly or indirectly) at the carboxyl (COOH or C) terminus of the heterologous food and/or feed enzyme moiety. In the chimeric proteins of formula (II), the carboxy terminus of the amino acid tether is located (directly or indirectly) at the amino (NH₂ or N) terminus of the heterologous food and/or feed enzyme moiety.

In yet another embodiment, in the chimeric proteins of formula (I) and (II), the food and/or feed enzyme can be a baking enzyme. In such embodiments, the chimeric protein can have having one of the following formulae (provided from the amino (NH₂) to the carboxyl (COOH) orientation):

$$B E - L - TT or \qquad ( Ia )$$

$$TT - L - BE \qquad ( IIa )$$

In both of these formulae, the residue “BE” refers to the heterologous baking enzyme moiety, the residue “L” refers to the presence of an optional linker while the residue “TT” refers to an amino acid tethering moiety. In the chimeric proteins of formula (Ia), the amino acid tether is located (directly or indirectly) at the carboxyl (COOH or C) terminus of the heterologous baking enzyme moiety. In the chimeric proteins of formula (IIa), the amino acid tether is located (directly or indirectly) at the amino (NH₂ or N) terminus of the heterologous food and/or feed enzyme moiety.

When the amino acid linker (L) is absent, the tethering amino acid moiety is directly associated with the heterologous food and/or feed enzyme (or with the heterologous baking enzyme). In the chimeras of formulae (I) and (Ia), this means that the carboxyl terminus of the heterologous food and/or feed enzyme moiety (or the carboxyl terminus of the heterologous baking enzyme moiety) is directly associated (with an amide linkage) to the amino terminus of the tethering amino acid moiety. In the chimeras of formulae (II) and (IIa), this means that the carboxyl terminus of the tethering amino acid moiety is directly associated (with an amide linkage) to the amino terminus of the heterologous food and/or feed enzyme (or of the heterologous baking enzyme).

In some embodiments, the presence of an amino acid linker (L) is desirable either to provide, for example, some flexibility between the heterologous food and/or feed enzyme moiety and the tethering amino acid moiety or to facilitate the construction of the heterologous nucleic acid molecule. As used in the present disclosure, the “amino acid linker” or “L” refer to a stretch of one or more amino acids separating the heterologous enzyme moiety FFE or BE and the amino acid tethering moiety TT (e.g., indirectly linking the heterologous food and/or feed enzyme to the amino acid tethering moiety TT). It is preferred that the amino acid linker be neutral, e.g., does not interfere with the biological (enzymatic) activity of the heterologous food and/or feed enzyme nor with the biological (cell-association) activity of the amino acid tethering moiety. In some embodiments, the amino acid linker L can increase the biological activity of the heterologous food and/or feed enzyme moiety and/or of the amino acid tethering moiety.

In instances in which the linker (L) is present in the chimeras of formulae (I) and (Ia), its amino end is associated (with an amide linkage) to the carboxyl end of the heterologous food and/or feed enzyme moiety and its carboxyl end is associated (with an amide linkage) to the amino end of the amino acid tethering moiety. In instances in which the linker (L) is present in the chimeras of formulae (II) and (IIa), its amino end is associated (with an amide linkage) to the carboxyl end of the amino acid tethering moiety and its carboxyl end is associated (with an amide linkage) to the amino end of the heterologous food and/or feed enzyme moiety.

Various amino acid linkers exist and include, without limitations, (G)ₙ, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker L is (GGGGS)ₙ (also referred to as G₄S) and, in still further embodiments, the amino acid linker L comprises more than one G₄S (SEQ ID NO: 41) motifs. For example, the amino acid linker L can be (G₄S)₃ and have the amino acid sequence of SEQ ID NO: 93. In another example, the amino acid linker L can be (G)₈ and have the amino acid sequence of SEQ ID NO: 94. In still another example, the amino acid linker L can be (G₄S)₈ and have the amino acid sequence of SEQ ID NO: 95.

The amino acid linker can also be, in some embodiments, GSAGSAAGSGEF (SEQ ID NO: 96).

Additional amino acid linkers exist and include, without limitations, (EAAK)ₙ and (EAAAK)ₙ, wherein n=is an integer between 1 to 8 (or more). In some embodiments, the one or more (EAAK)ₙ/(EAAAK)ₙ motifs can be separated by one or more additional amino acid residues. In an embodiment, the amino acid linker comprises one or more EA₂K (SEQ ID NO: 100) or EA₃K (SEQ ID NO: 101) motifs. In an embodiment, the amino acid linker can be (EAAK)₃ and has the amino acid sequence of SEQ ID NO: 97. In another embodiment, the amino acid linker can be (A(EAAAK)₄ALEA(EAAAK)₄A) and has the amino acid sequence of SEQ ID NO: 99.

Further amino acid linkers include those having one or more (AP)ₙ motifs wherein n=is an integer between 1 to 10 (or more). In an embodiment, the linker is (AP)₁₀ and has the amino acid of SEQ ID NO: 98.

In some embodiments, the linker also includes one or more HA tag (SEQ ID NO: 53).

### Tools for Making the Recombinant Yeast Host Cell

In order to make the recombinant yeast host cells, heterologous nucleic acid molecules (also referred to as expression cassettes) are made in vitro and introduced into the yeast host cell in order to allow the recombinant expression of the heterologous food and/or feed enzyme.

The heterologous nucleic acid molecules of the present disclosure comprise a coding region for the heterologous polypeptide, e.g., the heterologous food and/or feed enzyme or a chimeric protein comprising same. A DNA or RNA “coding region” is a DNA or RNA molecule (preferably a DNA molecule) which is transcribed and/or translated into an heterologous food and/or feed enzyme in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

In some embodiments, the heterologous nucleic acid molecules of the present disclosure include a promoter as well as a coding sequence for an heterologous food and/or feed enzyme (including chimeric proteins comprising same). The heterologous nucleic acid sequence can also include a terminator. In the heterologous nucleic acid molecules of the present disclosure, the promoter and the terminator (when present) are operatively linked to the nucleic acid coding sequence of the heterologous food and/or feed enzyme (including chimeric proteins comprising same), e.g., they control the expression and the termination of expression of the nucleic acid sequence of the heterologous food and/or feed enzyme (including chimeric proteins comprising same). The heterologous nucleic acid molecules of the present disclosure can also include a nucleic acid coding for a signal peptide, e.g., a short peptide sequence for exporting the heterologous food and/or feed enzyme outside the host cell. When present, the nucleic acid sequence coding for the signal peptide is directly located upstream and is in frame with the nucleic acid sequence coding for the heterologous food and/or feed enzyme (including chimeric proteins comprising same).

In the heterologous nucleic acid molecule described herein, the promoter and the nucleic acid molecule coding for the heterologous food and/or feed enzyme (including chimeric proteins comprising same) are operatively linked to one another. In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous protein from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. In the context of the present disclosure, it is important that the selected promoter allows the expression of the heterologous nucleic acid molecule during the propagation phase of the recombinant yeast host cell in order to allow a sufficient amount of cell-associated heterologous food and/or feed enzymes to be expressed. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promoter or a combination of different promoters.

In the present disclosure, promoters allowing or favoring the expression of the heterologous proteins during the propagation phase of the recombinant yeast host cells are preferred. Yeasts that are facultative anaerobes, are capable of respiratory reproduction under aerobic conditions and fermentative reproduction under anaerobic conditions. In many commercial applications, yeast are propagated under aerobic conditions to maximize the conversion of a substrate to biomass. Optionally, the biomass can be used in a subsequent fermentation under anaerobic conditions to produce a desired metabolite. In the context of the present disclosure, it is important that the promoter or combination of promoters present in the heterologous nucleic acid is/are capable of allowing the expression of the heterologous food and/or feed enzyme or its corresponding chimera during the propagation phase of the recombinant yeast host cell. This will allow the accumulation of the heterologous food and/or feed enzyme associated with the recombinant yeast host cell prior to fermentation (if any). In some embodiments, the promoter allows the expression of the heterologous food and/or feed enzyme or its corresponding chimera during propagation, but not during fermentation (if any) of the recombinant yeast host cell.

The promoters can be native or heterologous to the heterologous gene encoding the heterologous protein. The promoters that can be included in the heterologous nucleic acid molecule can be constitutive or inducible promoters (such as those described in Perez-Torrado et al., 2005). Inducible promoters include, but are not limited to glucose-regulated promoters (e.g., the promoter of the hxt7 gene (referred to as hxt7p) and having the nucleic acid sequence of SEQ ID NO: 30, a functional variant or a functional fragment thereof; the promoter of the ctt1 gene (referred to as ctt1p) and having the nucleic acid sequence of SEQ ID NO: 60, a functional variant or a functional fragment thereof; the promoter of the glo1 gene (referred to as glo1p) and having the nucleic acid sequence of SEQ ID NO: 59, a functional variant or a functional fragment thereof; the promoter of the ygp1 gene (referred to as ygp1p) and having the nucleic acid sequence of SEQ ID NO: 61, a functional variant or a functional fragment thereof; the promoter of the gsy2 gene (referred to as gsy2p) and having the nucleic acid sequence of SEQ ID NO: 53, a functional variant or a functional fragment thereof), molasses-regulated promoters (e.g., the promoter of the mol1 gene (referred to as mol1p) described in Praekelt et al., 1992 or having the nucleic acid sequence of SEQ ID NO: 64, a functional variant or a functional fragment thereof), heat shock-regulated promoters (e.g., the promoter of the glo1 gene (referred to as glo1p) and having the nucleic acid sequence of SEQ ID NO: 59, a functional variant or a functional fragment thereof; the promoter of the sti1 gene (referred to as sti1p) and having the nucleic acid sequence of SEQ ID NO: 56, a functional variant or a functional fragment thereof; the promoter of the ygp1 gene (referred to as ygp1p) and having the nucleic acid sequence of SEQ ID NO: 61, a functional variant or a functional fragment thereof; the promoter of the gsy2 gene (referred to as gsy2p) and having the nucleic acid sequence of SEQ ID NO: 53, a functional variant or a functional fragment thereof), oxidative stress response promoters (e.g., the promoter of the cup1 gene (referred to as cup1p) and having the nucleic acid sequence of SEQ ID NO: 58, a functional variant or a functional fragment thereof; the promoter of the ctt1 gene (referred to as ctt1p) and having the nucleic acid sequence of SEQ ID NO: 60, a functional variant or a functional fragment thereof; the promoter of the trx2 gene (referred to as trx2p) and having the nucleic acid sequence of SEQ ID NO: 55, a functional variant or a functional fragment thereof; the promoter of the gpd1 gene (referred to as gpd1p) and having the nucleic acid sequence of SEQ ID NO: 57, a functional variant or a functional fragment thereof; the promoter of the hsp12 gene (referred to as hsp12p) and having the nucleic acid sequence of SEQ ID NO: 63, a functional variant or a functional fragment thereof), osmotic stress response promoters (e.g., the promoter of the ctt1 gene (referred to as ctt1p) and having the nucleic acid sequence of SEQ ID NO: 60, a functional variant or a functional fragment thereof; the promoter of the glo1 gene (referred to as glo1p) and having the nucleic acid sequence of SEQ ID NO: 59, a functional variant or a functional fragment thereof; the promoter of the gpd1 gene (referred to as gpd1p) and having the nucleic acid sequence of SEQ ID NO: 57, a functional variant or a functional fragment thereof; the promoter of the ygp1 gene (referred to as ygp1p) and having the nucleic acid sequence of SEQ ID NO: 61, a functional variant or a functional fragment thereof) and nitrogen-regulated promoters (e.g., the promoter of the ygp1 gene (referred to as ygp1p) and having the nucleic acid sequence of SEQ ID NO: 61, a functional variant or a functional fragment thereof).

Promoters that can be included in the heterologous nucleic acid molecule of the present disclosure include, without limitation, the promoter of the tdh1 gene (referred to as tdh1p and having, for example, the nucleic acid sequence of SEQ ID NO: 27, a functional variant or a functional fragment thereof), of the hor7 gene (referred to as hor7p and having, for example, the nucleic acid sequence of SEQ ID NO: 28, a functional variant or a functional fragment thereof), of the hsp 150 gene (referred to as hsp150p and having, for example, the nucleic acid sequence of SEQ ID NO: 29, a functional variant or a functional fragment thereof), of the hxt7 gene (referred to as hxt7p and having, for example, the nucleic acid sequence of SEQ ID NO: 30, a functional variant or a functional fragment thereof), of the gpm1 gene (referred to as gpm1p and having, for example, the nucleic acid sequence of SEQ ID NO: 31, a functional variant or a functional fragment thereof), of the pgk1 gene (referred to as pgk1p and having, for example, the nucleic acid sequence of SEQ ID NO: 32, a functional variant or a functional fragment thereof) and/or of the stl1 gene (referred to as stl1p and having, for example, the nucleic acid sequence of SEQ ID NO: 33, a functional variant or a functional fragment thereof). In an embodiment, the promoter is or comprises the tdh1p and/or the hor7p. In still another embodiment, the promoter comprises or consists essentially of the tdh1p and the hor7p. In a further embodiment, the promoter is the thd1p.

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous food and/or feed enzyme or its chimera during the propagation phase of the recombinant yeast host cells. Usually, functional fragments are either 5′ and/or 3′ truncation of one or more nucleic acid residue from the native promoter nucleic acid sequence.

In some embodiments, the nucleic acid molecules include a one or a combination of terminator sequence(s) to end the translation of the heterologous food and/or feed enzyme (or of the chimeric protein comprising same). The terminator can be native or heterologous to the nucleic acid sequence encoding the heterologous food and/or feed enzyme or its corresponding chimera. In some embodiments, one or more terminators can be used. In some embodiments, the terminator comprises the terminator from is from the dit1 gene (referred to as dit1t and can have, for example, the nucleic acid sequence of SEQ ID NO: 34, a functional variant or a functional fragment thereof), from the idp1 gene (referred to as idp1t and can have, for example, the nucleic acid sequence of SEQ ID NO: 35, a functional variant or a functional fragment thereof), from the gpm1 gene (referred to as gpm1t and can have, for example, the nucleic acid sequence of SEQ ID NO: 36, a functional variant or a functional fragment thereof), from the pma1 gene (referred to as pma1t and can have, for example, the nucleic acid sequence of SEQ ID NO: 37, a functional variant or a functional fragment thereof), from the tdh3 gene (referred to as tdh3t and can have, for example, the nucleic acid sequence of SEQ ID NO: 38, a functional variant or a functional fragment thereof), from the hxt2 gene (referred to as hxt2t and can have, for example, the nucleic acid sequence of SEQ ID NO: 39, a functional variant or a functional fragment thereof), from the adh3 gene (referred to as adh3t and can have, for example, the nucleic acid sequence of SEQ ID NO: 70, a functional variant or a functional fragment thereof) and/or from the ira2 gene (referred to as ira2t and can have, for example, the nucleic acid sequence of SEQ ID NO: 40, a functional variant or a functional fragment thereof). In an embodiment, the terminator is derived from the dit1 gene (and can have, for example, the nucleic acid sequence of SEQ ID NO: 34, a functional variant or a functional fragment thereof). In another embodiment, the terminator comprises or is derived from the adh3 gene (and can have, for example, the nucleic acid sequence of SEQ ID NO: 70, a functional variant or a functional fragment thereof). In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera.

In some embodiments, the heterologous nucleic acid molecules include a coding sequence for one or a combination of signal peptide sequence(s) allowing the export of the heterologous protein (or of the chimeric protein comprising same) outside the yeast host cell's wall. The signal peptide sequence can simply be added to the nucleic acid molecule (usually in frame with the sequence encoding the heterologous food and/or feed enzyme) or replace the signal peptide sequence already present in the heterologous food and/or feed enzyme. The signal peptide sequence can be native or heterologous to the nucleic acid sequence encoding the heterologous food and/or feed enzyme or its corresponding chimera. In some embodiments, one or more signal sequences can be used. In some embodiments, the signal sequence is from the gene encoding the invertase protein (and can have, for example, the amino acid sequence of SEQ ID NO: 68, a variant thereof or a fragment thereof), the AGA2 protein (and can have, for example, the amino acid sequence of SEQ ID NO: 69, a variant thereof or a fragment thereof) or the fungal amylase protein (and can have, for example, the amino acid sequence of SEQ ID NO: 107, a variant thereof or a fragment thereof). In the context of the present disclosure, the expression “functional variant of a signal sequence” refers to a nucleic acid sequence that has been substituted in at least one nucleic acid position when compared to the native signal sequence which retain the ability to direct the expression of the heterologous food and/or feed enzyme or its corresponding chimera outside the cell. In the context of the present disclosure, the expression “functional fragment of a signal sequence” refers to a shorter nucleic acid sequence than the native signal sequence which retain the ability to direct the expression of the heterologous food and/or feed enzyme or its corresponding chimera outside the cell.

In some embodiments in which it is desirable to express the heterologous food and/or feed enzyme inside the recombinant yeast host cell (intracellularly), the heterologous nucleic acid molecule can exclude the portion coding for the signal peptide sequence which is found in the native gene encoding the food and/or feed enzyme.

The heterologous nucleic acid molecule encoding the heterologous food and/or feed enzyme, chimera, variant or fragment thereof can be integrated in the genome of the yeast host cell. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

The present disclosure also provides nucleic acid molecules for modifying the yeast host cell so as to allow the expression of the heterologous food and/or enzymes, chimeras, variants or fragments thereof. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the encoded food and/or feed enzymes, chimeras, variants or fragments.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The present disclosure also provides nucleic acid molecules that are hybridizable to the complement nucleic acid molecules encoding the heterologous polypeptides as well as variants or fragments. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acid molecules contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived. For hybridizations with shorter nucleic acids, i.e., oligonucleotides, the position of mismatches becomes more important, and the length of the oligonucleotide determines its specificity. In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

### Processes for Propagating and Formulating the Recombinant Yeast Host Cell

The present disclosure allows for making a yeast composition comprising the recombinant yeast host cell of the present disclosure. In some embodiments, the yeast composition can be used to reduce or waive the requirement of supplementing a food or feed-making process with exogenous (and purified/isolated) enzymes.

The process for making the yeast composition broadly comprises two steps: a first step of propagating the recombinant yeast host cell and a second step of formulating the yeast composition. As used in the context of the present disclosure, a “yeast composition” is a composition comprising the recombinant yeast host cell of the present disclosure which has been propagated. The yeast composition can be used, for example, in a following fermentation (to provide the heterologous enzyme in situ during fermentation) or to make a food/feed product. In an embodiment, the recombinant yeast host cell is provided in an active or in a semi-active form in the yeast composition. For example, an embodiment of the yeast composition is a cream yeast made from the recombinant yeast host cell of the present disclosure.

The propagation step can be a continuous culture, a batch culture or a fed-batch culture. In the propagation step, the recombinant yeast host cell is placed in a culture medium which can, in some embodiments, allow for rapid growth. For example, the culture medium can comprise a carbon source (such as, for example, molasses, sucrose, glucose, dextrose syrup, ethanol and/or corn steep liquor), a nitrogen source (such as, for example, ammonia) and a phosphorous source (such as, for example, phosphoric acid). The propagation step can be subdivided into two steps, an initial seeding step and a further large-scale propagation step. During the propagation step, it is possible to monitor and adjust the temperature (usually at about 32° C. when the recombinant yeast host cell is from the species Saccharomyces cerevisiae), the pH and the aeration conditions to favor or optimize the division of the recombinant yeast host cell. For example, when fed-batch propagation conditions are employed, using high aeration and incremental carbohydrate addition can optimize the yield of the biomass of the yeast composition.

In the formulating step, the mixture obtained after propagation (comprising the propagated recombinant yeast host cell(s)) can be modified. One of the advantages of the recombinant yeast host cells of the present disclosure is that the heterologous food/feed enzyme activity is associated with the recombinant yeast host cell that therefore concentrating the biomass after propagation will also increase the amount/activity of the heterologous food/feed enzyme. In an embodiment for providing a yeast composition, at least one component of the mixture obtained after propagation is removed from the culture medium to provide the yeast composition. This component can be, without limitation, water, amino acids, peptides and proteins, nucleic acid residues and nucleic acid molecules, cellular debris, fermentation products, etc. In an embodiment, the formulating step comprises substantially isolating the propagated yeast recombinant host cells (e.g., the biomass) from the components of the culture medium. As used in the context of the present disclosure, the expression “substantially isolating” refers to the removal of the majority of the components of the culture medium from the propagated recombinant yeast host cells. In order to provide the yeast composition, the propagated recombinant yeast host cells can be centrifuged (and the resulting cellular pellet comprising the propagated recombinant yeast host cells can optionally be washed), filtered and/or dried (optionally using a vacuum-drying technique). The isolated recombinant yeast host cells can then be formulated in a yeast composition. The formulation step can, in some embodiments, preserve the viability (at least in part) of the recombinant yeast host cells. As such, the yeast composition can be provided in an active or a semi-active form. The yeast composition can be provided in a liquid, semi-solid or dry form. In an embodiment, the yeast composition can be provided in the form of a cream yeast.

The yeast composition can further be modified into a yeast product. As used in the context of the present disclosure, a yeast product is a product obtained from the propagated recombinant yeast host cell which comprises the heterologous food and/or feed enzyme. The yeast product can be, for example, a yeast lysate (e.g., an autolysate), a yeast extract, a yeast fraction (e.g., yeast cell walls) and/or the heterologous food and/or feed enzyme in a substantially isolated form. As used in the context of the present disclosure, the expression “substantially isolating/purifying the heterologous food and/or feed enzymes from the lysed recombinant yeast host cells” refers to the removal of the majority of the components of the lysed recombinant yeast host cells from the heterologous food and/or feed enzymes and providing same in an isolated/purified form.

The yeast composition as well as the yeast product can be provided as a food additive. As used in the present disclosure, the expression “food additive” refers to a product used in human nutrition for purposes of improving the quality of food or to improve the production process. In such embodiment, the yeast composition can also include, without limitation, a carrier (such as for example, salt or a wheat grit), a stabilizing agent and/or an oil. In a specific embodiment, the yeast composition can be provided as a live yeast composition (such as, for example, a yeast cream) suitable for downstream food preparation, as an inactivated yeast composition, as a yeast fraction and/or as a purified food enzyme. In another specific embodiment, the yeast composition can be provided as a dried preparation (spray-dried for example) suitable for downstream food preparation.

The yeast composition can be provided as a feed additive. As used in the present disclosure, the expression “feed additive” refers to a product used in animal nutrition for purposes of improving the quality of feed, the quality of food from animal origin and/or to improve the animals' performance and health (e.g., providing enhanced digestibility of the feed materials). In such embodiment, the yeast composition can also include, without limitation, a carrier (such as for example, salt or a wheat grit), a stabilizing agent and/or an oil. In a specific embodiment, the yeast composition can be provided as a live yeast composition (such as, for example, a yeast cream) suitable for downstream feed preparation, as an inactivated yeast composition, as a yeast fraction and/or as a purified feed enzyme. In another specific embodiment, the yeast composition can be provided as a dried preparation (spray-dried for example) suitable for downstream feed preparation. In an embodiment, the feed additive is added to the animal's diet to supplement it.

### Processes for Making Food and Feed Products

The recombinant yeast host cell of the present disclosure have been designed to be used in the preparation of products for human (food) or animal (feed) consumption. The present disclosure thus provides a process comprising including the recombinant yeast host cell of the present disclosure in the food or feed product. In some embodiments, it may be advantageous to provide the recombinant yeast host cell of the present disclosure as a food additive or as a feed additive. In some embodiments, the process can also include fermenting the product and/or baking the food or feed product. In instances in which the process includes a fermentation step, the fermentation can be conducted (totally or in part) in the presence of or by the recombinant yeast host cell described herein. The process of the present disclosure can be used to extend the shelf-life of the food or feed products. The enzymatic activity (associated with the heterologous food and/or feed enzyme as well as chimeric proteins comprising same) of the recombinant yeast cells can be dosed prior to use and adjusted depending on the type of activity warranted.

In an embodiment, the food and feed products are baked products. In such embodiment, a recombinant yeast host cell expressing the cell-associated baking enzyme is preferably used. Baked products such as yeast-leavened baked products can be fermented by the recombinant yeast host cell described herein. Yeast-leavened baked products include, without limitation, bread, pastries (including croissants), rolls, pita, tortillas, bagels and pie or pizza crusts and the like. When used during the process for making yeast-leavened products, the recombinant yeast host cells can be the sole fermenting organism that is added to the fermentable substrate. In other instances, the recombinant yeast host cells can be admixed with non-recombinant (e.g., wild-type) yeasts up to provide the adequate dose of heterologous baking enzyme activity. For example, the recombinant yeast host cell (which can be a recombinant Saccharomyces cerevisiae yeast host cell) can be combined in any ratio with a wild-type yeast host cell (which can be a wild-type non-recombinant Saccharomyces cerevisiae). In an embodiment, the ratio between recombinant:wild-type is between 1:100 and 100:1.

Amylolytic enzymes are of particular interest in the production of yeast-leavened baked products because they favor the hydrolysis of starch (either in a raw or hydrolyzed form) and therefore provide an energy source to the fermenting yeasts to accelerate the fermentation process, increase CO₂ production, increase ethanol production and/or improve the organoleptic properties of the fermented product. Maltogenic amylases are, in particular, very useful in the process for making bread because they are known to extend the shelf-life by maintaining the softness and the resilience of baked bread.

In another embodiment, the baked products are not fermented by the recombinant yeast host cell described herein and are instead chemically leavened or unleavened. Chemically leavened and unleavened baked products include, without limitation, cakes and flatbreads.

In the process described herein, the recombinant yeast host cells of the present disclosure can be provided in an active form (e.g., liquid, compressed, or fluid-bed dried yeast), in a semi-active form (e.g., liquid, compressed, or fluid-bed dried), in an inactive form (e.g., drum- or spray-dried) as well as a mixture therefore. For example, the recombinant yeast host cells can be a combination of active and semi-active or inactive forms to provide the ratio and dose of the baking enzyme required for making baked products.

The present invention will be more readily understood by referring to the following examples which are given to illustrate the invention rather than to limit its scope.

### Example I—Material and Methods

TABLE 1
Description of the yeast strains used in the examples. These strains were constructed with expression cassettes, integrated into
the FCY1 locus on each chromosome, the number of copies is provided in the table. The original strain background used for each
strain is also provided in the table. Each integrated cassette included a copy of an heterologous enzyme, one or more promoter
and one or more terminator. In some instances, the signal peptide of the heterologous enzyme has been replaced by another signal
peptide as indicated in the table. When the heterologous enzyme is expressed in a tethered form, the geometry in of the tether
is provided (see definition of formula I and II above) and the linker as well as the tether are provided. N.A. = not applicable.

| | | | Copies of | | | | | | |
|---|---|---|---|---|---|---|---|---|---|
| | | | heterologous | | | | | | |
| | | | enzyme | | | | | | |
| | Heterologous | Original | integrated | | | | | | |
| | enzyme | strain | per | | | Type of | Signal | | |
| Name | expressed | background | chromosome | Promoter | Terminator | expression | peptide¹ | Linker² | Tether³ |
| M2390 | None | N.A. | N.A. | N.A. | N.A. | N.A. | N.A. | N.A. | N.A. |
| (Saccharomyces | | | | | | | | | |
| cerevisiae) | | | | | | | | | |
| M8498 | Glucoamylase | M10474 | 1 | TEF2p | SED1t | Free | Invertase | None | None |
| | (SEQ ID | | | | | secreted | | | |
| | NO: 29) | | | | | | | | |
| M10074 | Alpha-amylase | M10474 | 1 | TEF2p | SED1t | Free | Invertase | None | None |
| | (SEQ ID | | | | | secreted | | | |
| | NO: 50) | | | | | | | | |
| M10474 | None | N.A. | N.A. | N.A. | N.A. | N.A. | N.A. | N.A. | N.A. |
| (Saccharomyces | | | | | | | | | |
| cerevisiae) | | | | | | | | | |
| M11312 | Phytase | M2390 | 1 | TEF2p | ADH3t | Free | Invertase | N.A. | N.A. |
| | (SEQ ID | | | | | secreted | | | |
| | NO: 67) | | | | | | | | |
| M12550 | None | N.A. | N.A. | N.A. | N.A. | N.A. | N.A. | N.A. | N.A. |
| (Saccharomyces | | | | | | | | | |
| cerevisiae) | | | | | | | | | |
| M12548 | None | N.A. | N.A. | N.A. | N.A. | N.A. | N.A. | N.A. | N.A. |
| (Saccharomyces | | | | | | | | | |
| boulardii) | | | | | | | | | |
| M12795 | Phytase | M12550 | 1 | TEF2p | ADH3t | Tethered - | Aga2 | (G₄S)₂ | Aga1/2 |
| | (SEQ ID | | | | | Formula (II) | | | (Aga2 |
| | NO: 67) | | | | | | | | on N- |
| | | | | | | | | | terminus |
| | | | | | | | | | of |
| | | | | | | | | | enzyme) |
| M12938 | Phytase | M12550 | 1 | TEF2p | ADH3t | Tethered - | Aga2 | (G₄S)₂ | Aga1/2 |
| | (SEQ ID | | | | | Formula (II) | | | |
| | NO: 67) | | | | | | | | |
| M12962 | None | N.A. | N.A. | N.A. | N.A. | N.A. | N.A. | N.A. | N.A. |
| (Saccharomyces | | | | | | | | | |
| cerevisiae var | | | | | | | | | |
| diastaticus) | | | | | | | | | |
| M13819 | Maltogenic | M10474 | 2 | TDH1p/HOR7p | DIT1t/IDP1t | Tethered - | Invertase | HA/G₄S | Spi1 |
| | alpha-amylase | | | | | Formula (I) | | | |
| | (SEQ ID | | | | | | | | |
| | NO: 51) | | | | | | | | |
| M13822 | Maltogenic | M10474 | 2 | TDH1p/HOR7p | DIT1t/IDP1t | Free | Invertase | None | None |
| | alpha-amylase | | | | | secreted | | | |
| | (SEQ ID | | | | | | | | |
| | NO: 51) | | | | | | | | |
| M13979 | Maltogenic | M10474 | 4 | TDH1p/HOR7p | DIT1t/IDP1t | Tethered - | Invertase | (G₄S)₂ | Spi1 |
| | alpha | | | | | formula (I) | | | |
| | amylase | | | | | | | | |
| | (SEQ ID | | | | | | | | |
| | NO: 51) | | | | | | | | |
| M14244 | Glucoamylase | M10474 | 1 | TEF2p | SED1t | Tethered - | Invertase | HA/G₄S | Sed1 |
| | (SEQ ID | | | | | formula (I) | | | |
| | NO: 29) | | | | | | | | |
| M14253 | Alpha-amylase | M10474 | 1 | TEF2p | SED1t | Tethered - | Invertase | HA/G₄S | Sed1 |
| | (SEQ ID | | | | | Formula (I) | | linker | |
| | NO: 50) | | | | | | | | |
| M14254 | Alpha-amylase | M10474 | 1 | TEF2p | SED1t | Tethered - | Invertase | None | Sed1 |
| | (SEQ ID | | | | | Formula (I) | | | |
| | NO: 50) | | | | | | | | |
| M14851 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| M15215 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 84 |
| | NO: 72) | | | | | | | | |
| M15222 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 74 |
| | NO: 71) | | | | | | | | |
| M15532 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 108) | | | | | | | | |
| M15771 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 78 |
| | NO: 71) | | | | | | | | |
| M15772 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 82 |
| | NO: 71) | | | | | | | | |
| M15773 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 86 |
| | NO: 72) | | | | | | | | |
| M15774 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 76 |
| | NO: 71) | | | | | | | | |
| M15775 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 92 |
| | NO: 72) | | | | | | | | |
| M15776 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 88 |
| | NO: 72) | | | | | | | | |
| M15777 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 80 |
| | NO: 71) | | | | | | | | |
| M15778 | Alpha-amylase | M2390 | 1 | TEF2p | ADH3t | Tethered - | Invertase | SEQ ID | SEQ ID |
| | (SEQ ID | | | | | Formula (I) | | NO: 94 | NO: 74 |
| | NO: 71) | | | | | | | | |
| M15779 | Alpha-amylase | M2390 | 1 | TEF2p | ADH3t | Tethered - | Invertase | SEQ ID | SEQ ID |
| | (SEQ ID | | | | | Formula (I) | | NO: 95 | NO: 74 |
| | NO: 71) | | | | | | | | |
| M15780 | Alpha-amylase | M2390 | 1 | TEF2p | ADH3t | Tethered - | Invertase | SEQ ID | SEQ ID |
| | (SEQ ID | | | | | Formula (I) | | NO: 97 | NO: 74 |
| | NO: 71) | | | | | | | | |
| M15781 | Alpha-amylase | M2390 | 1 | TEF2p | ADH3t | Tethered - | Invertase | SEQ ID | SEQ ID |
| | (SEQ ID | | | | | Formula (I) | | NO: 98 | NO: 84 |
| | NO: 72) | | | | | | | | |
| M15782 | Alpha-amylase | M2390 | 1 | TEF2p | ADH3t | Tethered - | Invertase | SEQ ID | SEQ ID |
| | (SEQ ID | | | | | Formula (I) | | NO: 95 | NO: 84 |
| | NO: 72) | | | | | | | | |
| M15784 | Alpha-amylase | M2390 | 1 | TEF2p | ADH3t | Tethered - | Invertase | SEQ ID | SEQ ID |
| | (SEQ ID | | | | | Formula (I) | | NO: 93 | NO: 84 |
| | NO: 71) | | | | | | | | |
| M15783 | Alpha-amylase | M2390 | 1 | TEF2p | ADH3t | Tethered - | Invertase | SEQ ID | SEQ ID |
| | (SEQ ID | | | | | Formula (I) | | NO: 99 | NO: 74 |
| | NO: 71) | | | | | | | | |
| M15785 | Alpha-amylase | M2390 | 1 | TEF2p | ADH3t | Tethered - | Invertase | SEQ ID | SEQ ID |
| | (SEQ ID | | | | | Formula (I) | | NO: 93 | NO: 84 |
| | NO: 72) | | | | | | | | |
| M15786 | Alpha-amylase | M2390 | 1 | TEF2p | ADH3t | Tethered - | Invertase | SEQ ID | SEQ ID |
| | (SEQ ID | | | | | Formula (I) | | NO: 94 | NO: 84 |
| | NO: 72) | | | | | | | | |
| M15787 | Alpha-amylase | M2390 | 1 | TEF2p | ADH3t | Tethered - | Invertase | SEQ ID | SEQ ID |
| | (SEQ ID | | | | | Formula (I) | | NO: 96 | NO: 74 |
| | NO: 71) | | | | | | | | |
| M15788 | Alpha-amylase | M2390 | 1 | TEF2p | ADH3t | Tethered - | Invertase | SEQ ID | SEQ ID |
| | (SEQ ID | | | | | Formula (I) | | NO: 98 | NO: 74 |
| | NO: 71) | | | | | | | | |
| M16221 | Alpha-amylase | M2390 | 1 | TEF2p | ADH3t | Tethered - | Invertase | SEQ ID | SEQ ID |
| | (SEQ ID | | | | | Formula (I) | | NO: 97 | NO: 84 |
| | NO: 72) | | | | | | | | |
| M16222 | Alpha-amylase | M2390 | 1 | TEF2p | ADH3t | Tethered - | Invertase | SEQ ID | SEQ ID |
| | (SEQ ID | | | | | Formula (I) | | NO: 99 | NO: 84 |
| | NO: 72) | | | | | | | | |
| M16251 | Alpha-amylase | M2390 | 1 | TEF2p | ADH3t | Tethered - | Invertase | HA/(G₄S)₃ | SEQ ID |
| | (SEQ ID | | | | | Formula (I) | | | NO: 90 |
| | NO: 72) | | | | | | | | |
| M16252 | Alpha-amylase | M2390 | 1 | TEF2p | ADH3t | Tethered - | Invertase | SEQ ID | SEQ ID |
| | (SEQ ID | | | | | Formula (I) | | NO: 96 | NO: 84 |
| | NO: 72) | | | | | | | | |
| M16273 | Glucose | M10474 | 2 | TDH1p/HOR7p | DIT1t/IDP1t | Intracellular | N.A. | N.A. | N.A. |
| | oxidase | | | | | | | | |
| | (SEQ ID | | | | | | | | |
| | NO: 103) | | | | | | | | |
| M16540 | Fungal | M10474 | 2 | TDH1p/HOR7p | DIT1t/IDP1t | Free | Fungal | N.A. | N.A. |
| | amylase | | | | | secreted | amylase | | |
| | (SEQ ID | | | | | | | | |
| | NO: 105) | | | | | | | | |
| M16772 | Fungal | M10474 | 2 | TDH1p/HOR7p | DIT1t/IDP1t | Free | Invertase | N.A. | N.A. |
| | amylase | | | | | secreted | | | |
| | (SEQ ID | | | | | | | | |
| | NO: 105) | | | | | | | | |
| M16780 | Glucose | M10474 | 2 | TDH1p/HOR7p | DIT1t/IDP1t | Free | Invertase | N.A. | N.A. |
| | oxidase | | | | | secreted | | | |
| | (SEQ ID | | | | | | | | |
| | NO: 103) | | | | | | | | |
| T2633 | Phytase | M12548 | 1 | TEF2p | ADH3t | Free | Invertase | N.A. | N.A. |
| | (SEQ ID | | | | | secreted | | | |
| | NO: 66) | | | | | | | | |
| T2634 | Phytase | M12548 | 1 | TEF2p | ADH3t | Tethered - | Invertase | (G₄S)₂ | Sed1 |
| | (SEQ ID | | | | | Formula (I) | | | |
| | NO: 66) | | | | | | | | |
| T2635 | Phytase | M12548 | 1 | TEF2p | ADH3t | Tethered - | Invertase | (G₄S)₂ | Tir1 |
| | (SEQ ID | | | | | Formula (I) | | | |
| | NO: 66) | | | | | | | | |
| T2636 | Phytase | M12548 | 1 | TEF2p | ADH3t | Tethered - | Invertase | (G₄S)₂ | Cwp2 |
| | (SEQ ID | | | | | Formula (I) | | | |
| | NO: 66) | | | | | | | | |
| T2637 | Phytase | M12548 | 1 | TEF2p | ADH3t | Tethered - | Invertase | (G₄S)₂ | Spi1 |
| | (SEQ ID | | | | | Formula (I) | | | |
| | NO: 66) | | | | | | | | |
| T2638 | Phytase | M12548 | 1 | TEF2p | ADH3t | Tethered - | Invertase | (G₄S)₂ | Pst1 |
| | (SEQ ID | | | | | Formula (I) | | | |
| | NO: 66) | | | | | | | | |
| T2705 | Phytase | M2390 | 1 | TEF2p | ADH3t | Tethered - | Aga2 | (G₄S)₂ | Aga1/2 |
| | (SEQ ID | | | | | Formula (II) | | | |
| | NO: 67) | | | | | | | | |
| T2706 | Phytase | M2390 | 1 | TEF2p | ADH3t | Tethered - | Invertase | (G₄S)₂ | Aga1/2 |
| | (SEQ ID | | | | | Formula (I) | | | |
| | NO: 67) | | | | | | | | |
| T2816 | Phytase | M12550 | 1 | TEF2p | ADH3t | Tethered - | Invertase | (G₄S)₂ | Sed1 |
| | (SEQ ID | | | | | Formula (I) | | | |
| | NO: 67) | | | | | | | | |
| T2986 | Maltogenic | M10474 | 2 | TDH1p/HOR7p | DIT1t/IDP1t | Tethered - | Invertase | HA/(G₄S)₂ | Flo1 |
| | alpha-amylase | | | | | Formula (I) | | | |
| | (SEQ ID | | | | | | | | |
| | NO: 51) | | | | | | | | |
| T2987 | Maltogenic | M10474 | 2 | TDH1p/HOR7p | DIT1t/IDP1t | Tethered - | Invertase | HA/(G₄S)₂ | Sed1 |
| | alpha-amylase | | | | | Formula (I) | | | |
| | (SEQ ID | | | | | | | | |
| | NO: 51) | | | | | | | | |
| T2988 | Maltogenic | M10474 | 2 | TDH1p/HOR7p | DIT1t/IDP1t | Tethered - | Invertase | HA/(G₄S)₂ | Tir1 |
| | alpha-amylase | | | | | Formula (I) | | | |
| | (SEQ ID | | | | | | | | |
| | NO: 51) | | | | | | | | |
| T2989 | Maltogenic | M10474 | 2 | TDH1p/HOR7p | DIT1t/IDP1t | Tethered - | Invertase | HA-(G₄S)₂ | Cwp2 |
| | alpha-amylase | | | | | Formula (I) | | | |
| | (SEQ ID | | | | | | | | |
| | NO: 51) | | | | | | | | |
| T2990 | Maltogenic | M10474 | 2 | TDH1p/HOR7p | DIT1t/IDP1t | Tethered - | Invertase | HA/(G₄S)₂ | Ccw1 |
| | alpha-amylase | | | | | Formula (I) | | | |
| | (SEQ ID | | | | | | | | |
| | NO: 51) | | | | | | | | |
| T2991 | Maltogenic | M10474 | 2 | TDH1p/HOR7p | DIT1t/IDP1t | Tethered - | Invertase | HA/(G₄S)₂ | Spi1 |
| | alpha-amylase | | | | | Formula (I) | | | |
| | (SEQ ID | | | | | | | | |
| | NO: 51) | | | | | | | | |
| T2994 | Maltogenic | M10474 | 2 | TDH1p/HOR7p | DIT1t/IDP1t | Free | Invertase | None | None |
| | alpha-amylase | | | | | secreted | | | |
| | (SEQ ID | | | | | | | | |
| | NO: 51) | | | | | | | | |
| T3892 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| T4328 | Maltogenic | M10474 | 2 | TDH1p/HOR7p | DIT1t/IDP1t | Tethered | Invertase | (G₄S)₂ | Spi1 |
| | alpha amylase | | | | | | | | |
| | (SEQ ID | | | | | | | | |
| | NO: 51) | | | | | | | | |
| T4329 | Maltogenic | M10474 | 2 | TDH1p/HOR7p | DIT1t/IDP1t | Free | Invertase | N.A. | N.A. |
| | alpha amylase | | | | | secreted | | | |
| | (SEQ ID | | | | | | | | |
| | NO: 51) | | | | | | | | |
| T4330 | Maltogenic | M10474 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |
| T4336 | Maltogenic | M12962 | 2 | TDH1p/HOR7p | DIT1t/IDP1t | Tethered - | Invertase | (G₄S)₂ | Spi1 |
| | alpha amylase | | | | | Formula (I) | | | |
| | (SEQ ID | | | | | | | | |
| | NO: 51) | | | | | | | | |
| T4337 | Maltogenic | M12962 | 2 | TDH1p/HOR7p | DIT1t/IDP1t | Free | Invertase | N.A. | N.A. |
| | alpha amylase | | | | | secreted | | | |
| | (SEQ ID | | | | | | | | |
| | NO: 51) | | | | | | | | |
| T4338 | Maltogenic | M12962 | 2 | TDH1p/HOR7p | DIT1t/IDP1t | Intracellular | N.A. | N.A. | N.A. |
| | alpha amylase | | | | | | | | |
| | (SEQ ID | | | | | | | | |
| | NO: 65) | | | | | | | | |

¹Invertase = SEQ ID NO: 52, Aga2 = SEQ ID NO: 69, fungal amylase = SEQ ID NO: 107
²HA = SEQ ID NO: 53: (G₄S)₂ = SEQ ID NO: 54
³Flo1 tether is a transmembrane domain located at the C-terminus = SEQ ID NO: 10; Sed1 tether is a GPI anchor located at the C-terminus = SEQ ID NO: 12; Tir1 tether is a mannoprotein GPI fragment located at the C-terminus = SEQ ID NO: 14; Cwp2 tether is a mannoprotein GPI fragment located at the C-terminus = SEQ ID NO: 16; Ccw12 tether is a mannoprotein GPI fragment located at the C-terminus = SEQ ID NO: 18; Spi1 tether is a GPI anchor located at the C-terminus = SEQ ID NO: 20; Pst1 tether is a GPI anchor = SEQ ID NO: 22; Aga1/2 tether, Aga2 disulfide bond to Aga1; Aga1 has GPI anchor, the enzyme is fused to Aga2 at the C-terminus = SEQ ID NO: 24: Aga1/2 tether, Aga2 disulfide bond to Aga1; Aga1 has GPI anchor, the enzyme is fused to Aga2 at the N-terminus = SEQ ID NO: 26.

Cell growth. Cells were grown overnight in 5 mL YPD (10 g/L yeast extract, 20 g/L bacteriological peptone, 40 g/L glucose). One (1) mL of whole culture as harvested and cells were pelleted by centrifugation. Cell-free supernatant was removed and saved for later analysis. Cell pellet was washed once and resuspended in deionized water.

Seed fed-batch fermentation. A molasses mixture was prepared (85% beet molasses, 15% cane molasses), diluted and its pH was adjust to pH 5.2 with sulfuric acid. The pure culture inoculum was diluted in sterile water and added to the molasses mixture with zinc sulfate, magnesium sulfate, biotin, thiamine, calcium pantothenate and phosphoric acid. The yeasts were propagated at 32° C., at pH 4.5, for 24 hours. The resulting propagated yeast were centrifuged and the propagation broth was washed using a laboratory Alfa Laval separator to reach approximately 20% yeast solids. The yeasts solids were treated with sulfuric acid and pH was adjusted afterwards with sodium hydroxide to provide the cream yeast.

Commercial fed-batch fermentation. A molasses mixture was prepared (85% beet molasses, 15% cane molasses), diluted and its pH was adjust to pH 5.2 with sulfuric acid. The cream yeast from the seed fed-batch was diluted in sterile water and added to the molasses mixture with zinc sulfate, magnesium sulfate, biotin, thiamine, calcium pantothenate. The resulting propagated yeast were centrifuged and the propagation broth was washed using a laboratory Alfa Laval separator to reach approximately 20% yeast solids.

Cream yeast and inactivated cream yeast. After the fermentation, the harvested fermentation broth was centrifuged and washed using a laboratory scale GEA separator to prepare yeast cream with a final dry weight close to 20%. To make the inactivated cream yeast, about 600 g of cream yeast was heated on a temperature controlled stirring/hot plate until 75° C. was reached. The cream was kept for 15 minutes at 75° C. and then removed from heat source.

Spray drying. Spray dried samples were prepared by drying at 150° C. with a mini spray dryer (Buchi B-290). Feeding rate was kept to maintain outlet temperature around 80-85° C.

Bead-milling/making bead-milled homogenate. Cream yeast was disrupted (with typical disruption efficiency of >95% of cells) by bead milling under the following bead mill conditions. Cream yeast (˜20% solids) was bead-milled with a Dyno KDL with 0.6 L chamber volume at 4° C., using 0.5-0.75 mm glass beads filling the chamber to 80% with 1.6 g/mL packing capacity and a 64 mm diameter agitator with peripheral speed of 10 m/s. The cream yeast flow rate was 6 kg/L/h.

Preparation of instant dried yeast (IDY). After the commercial fermentations targeting for the production of IDY samples, the harvested broth was centrifuged and washed using a laboratory scale GEA separator to prepare yeast cream with a final dry weight close to 20%. The cream was then filtered in a vacuum filtration system to make cake yeast. To remove additional water, the yeast cake was further pressed to achieve a dry weight of about 35% before extrusion. The pressed cake was then extruded after well mixed with span for 5 minutes. The span addition rate was 1% on yeast dry matter basis. After extrusion, the yeast was dried in a lab-scale fluidized-bed dryer (Aeromatic AG). The drying temperature was set and controlled at 35-40° C. The drying lasted about 20-25 minutes to achieve a solids content of more than 94%. In term of the fermentation recipes, the significant difference for the IDY fermentation recipe is that it has a 2 hrs maturation period towards end of the fermentation, in which ammonia (N) is stopped and fermentation temperature is increased to 35° C.

Fermenter autolysis. At least 3 L (minimum working volume) of cream at 20% solids was transferred into a 20 L fermenter (BIOENGINEERING). Autolysis was performed at 55° C. and pH 5.5 (automated pH control with 2N sulfuric acid) with a gentle agitation at 70 rpm. Autolysate (˜20% dry weight) was harvested after a 24 hours incubation and separated as described below.

Lab scale autolysis. This autolysis is similar to the fermenter autolysis described above, but was performed at a smaller scale and with slightly different parameters. The cream yeast (20% solids) was submitted to autolysis and the pH was adjusted to pH 7. The mixture was incubated in 50 mL conical tubes in a 55° C. water bath for 48 hours.

Separation of autolysate without washing. After fermenter autolysis, the total autolysate was separated at 11,000 RCF for 10 minutes in 1 L bottles in a Sorvall Lynx 6000 centrifuge to obtain a soluble fraction (11-13% dry weight, yeast extract) and insoluble fraction (yeast cell wall). Dry weight and enzyme activity were measured for the total autolysate, yeast extract and cell wall fractions for dry weight and MANU balances.

Separation of autolysate with washing. Separations were performed by centrifuging fermenter autolysate in 50 ml conical tubes for 10 minutes at 3,000 RCF. Two additional washes were performed by adding water equal to the weight of supernatant obtained from the centrifuge step. YE (yeast extract) separation yield is calculated as the recovery of solids from separation only (WF=0) and of separation plus one or two washes (WF=1 or 2, respectively), relative to the starting solids in the autolysate. YE MANU recovery is calculated as the activity (in Phadebas MANU) from separation only (WF=0) and of separation plus one or two washes (WF=1 or 2), relative to starting total Phadebas MANU in the autolysate.

Ultrafiltration. Fermenter autolysate was separated by centrifuging in 1 L bottles at 11,000 RCF and the yeast extract fraction was further concentrated by ultrafiltration with a 10 kDa molecular weight cutoff PES membrane (Millipore, Biomax-10). The retentate fraction is retained by the membrane and permeate fraction passes through the membrane.

Maltogenic amylase assay. One Maltogenic Amylase Novo Unit, MANU, is the amount of enzyme which under standard conditions will cleave one micromol maltotriose per minute. Prior to assaying for enzymatic activity, cream yeast samples were inactivated by incubation at 60° C. for 10 minutes in MANU assay buffer (0.1 M citric acid, pH 5.0). Samples were then mixed with 20 mg/ml maltotriose substrate and incubated at 37° C. for 30 minutes. Reactions were stopped by addition of an equal volume of 1 N sodium hydroxide stop reagent. Glucose hydrolyzed by maltogenic amylase activity was measured after a 15-minute room temperature incubation with glucose (HK) assay reagent (Sigma G3293). Absorbance was read at 340 nm in a spectrophotometer. Unknown samples were compared to a dose curve of Novamyl® with known enzyme activity. This method was applied to generate the results of FIG. 1 only.

Phadebas MANU enzyme activity assay. Phadebas tablets contain a water insoluble starch substrate and a blue dye, bound to the dye with crosslinks. The substrate is hydrolyzed by maltogenic amylase, releasing blue dye which is soluble. After terminating the reaction and centrifuging, the absorbance of the solution was measured spectrophotometrically and is considered a proxy for enzyme activity. For each sample, one Phadebas tablet was added to 4.9 mL of citrate-phosphate buffer (70 mM disodium hydrogen phosphate, 30 mM citric acid, pH 5.5), incubated in a 60° C. water bath for 5 minutes. Then, 0.1 mL of standard or sample, diluted in citrate-phosphate buffer, was added to the tablet and buffer solution and incubated for 15 minutes in the 60° C. water bath. The reaction was terminated by adding 1 mL of 0.5 M sodium hydroxide solution and mixing. The tubes were centrifuged to remove solids and absorbance of the substrate was measured at 620 nm with a spectrophotometer. Samples (dry or liquid) are compared to a dose curve of Novamyl® with known activity. This methods was applied to generate all of the MANU results, except for FIG. 1.

Glucose oxidase assay. Cells were grown in batch in yeast extract peptone media plus 2% glucose at 30° C. for 24 hours. To obtain the disrupted washed cell supernatant, the cells were dead-beaten with glass beads 2×1 min in assay buffer, with one minute rest between. The supernatant was separated from the whole lysate by centrifugation. Whole culture, supernatant, disrupted washed cell supernatant (which reflects the intracellular cell-associated activity), washed cells or a positive control of Gluzyme® (2.40 GODU/mL corresponding to 10 000 BG) were measured with the K-GLOX™ kit (Megazyme): samples in assay buffer (100 mM potassium phosphate, pH 7, containing 0.5 mg/mL BSA and 0.02% (w/v) sodium azide) were mixed with 90 mg/ml glucose and POD mixture and incubated at room temperature for 20 minutes. Absorbance was measured with a spectrophotometer at 510 nm.

Alpha-amylase assay (FIG. 5). Alpha-amylase activity was measured by adding 25 μL washed cells or cell free supernatant to 25 μL 5 mM p-Nitrophenyl α-D-hexaoside in 50 mM sodium acetate pH 5. The reaction was incubated at 35° C. for 2 hours and terminated by the addition of 50 μL 1M sodium bicarbonate. Cells were pelleted, 50 μL of the assay mixture was transferred to a microtiter plate and absorbance at 405 nm was measured. Activity of the cell fraction was represented as a percentage of the total activity (“bound”+“free”).

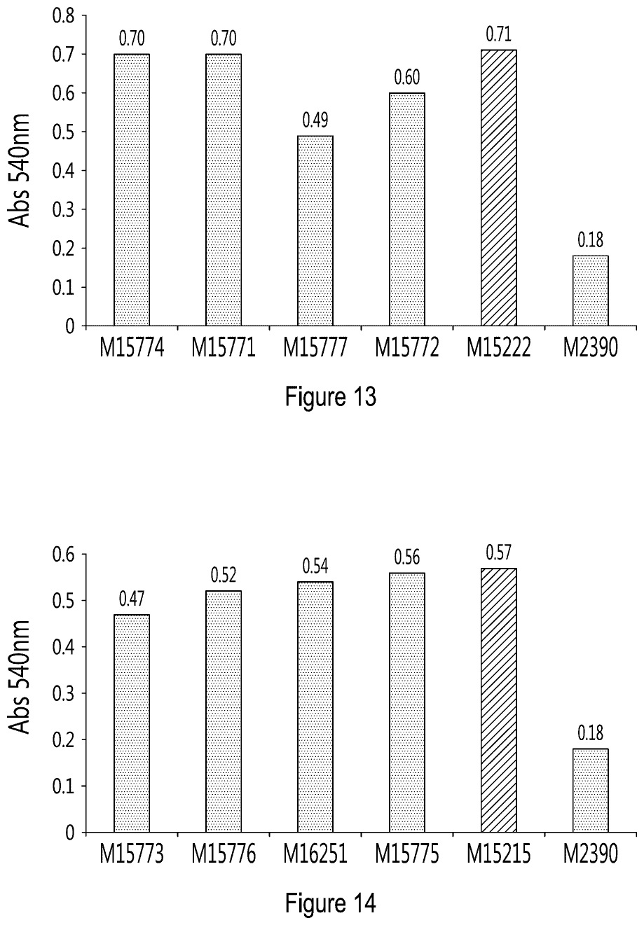

Alpha-amylase assay (FIGS. 13 to 16). The strains were initially grown in 600 μL of YPD40 at 35° C. for 48 h in 96-well plates on a shaker at 900 rpm. Alpha-amylase activity was determined by adding 25 μL of washed cells or cell-free supernatant to 100 μL of 1% raw starch with 50 mM sodium acetate buffer (pH 5.2). The assay was treated for 30 min at 85° C. using an Eppendorf Gradient Cycler. The reducing sugars were measured using the Dinitrosalicylic Acid Reagent Solution (DNS) method, using a 2:1 DNS:starch assay ratio and boiled at 100° C. for 5 min. The absorbance was measured at 540 nm.

Fungal amylase activity. Cells were grown in batch in yeast extract peptone media plus 2% glucose at 30° C. for 24 hours. Whole culture, supernatant, and either disrupted cell supernatant or washed cells were resuspended in assay buffer (70 mM disodium hydrogen phosphate, 30 mM citric acid, pH 5.5) were mixed with 1% gelatinized wheat starch in assay buffer and incubated at 30° C. for 1 hour. 3,5-Dinitrosalicylic acid (DNS) was added to react with reducing ends and boiled at 99° C. for 5 minutes. Absorbance was measured with a spectrophotometer at 540 nm.

Wheat starch activity assay. Cells were grown in batch in yeast extract peptone media plus 4% glucose at 35° C. for 48 hours. Whole culture, supernatant and washed cells resuspended in assay buffer (50 mM sodium acetate, pH 5) were mixed with 1% wheat starch in assay buffer and incubated at 60° C. for 5 minutes. Then, 3,5-dinitrosalicylic acid was added to react with reducing ends and boiled at 99° C. for 5 minutes. Absorbance was measured with a spectrophotometer at 540 nm.

Phytase activity assay. A 2-fold serial dilution of 1 M potassium phosphate monobasic was prepared as a standard for calculating FTUs. 190 μl of 5 mM sodium phytate solution pH 5.5 was added to each well of a 96 well PCR plate. Standards or supernatants of overnight cultures of yeast in yeast extract peptone media with 4% glucose were combined with 5 mM sodium phytate solution pH 5.5 and were incubated at 37° C. for 30 min. Cell associated samples were measured again following 2 hours of incubation. Equal volumes of reaction and color change solution (4 parts reagent A to 1 part reagent B, where reagent A is 12 mM ammonium heptamolybdate-HCl in water and reagent B is 2.7% ferrous sulfate in water) were combined and incubated for 10 minutes at room temperature before pelleting at 3500 rpm for 3 minutes. Absorbance of each sample or standard was read at 700 nm in a spectrophotometer

Bake test with Novamyl® as a control. Bread was made with the M1074 strain in the presence and absence of externally added doses of the Novamyl® maltogenic amylase product (as indicated in the figures) and was compared to bread made with strains expressing a cell-associated MAA (in the absence of Novamyl®). For bread made with cream yeast expressing MAA, doses of cream yeast were normalized to 1 000 MANU based on the enzyme activity shown in FIG. 1B and supplemented with wild-type C strain cream yeast for sufficient gassing power. Briefly, 1 000 g of white flour, 600 g of water, 35 g of cream yeasts (dosed at 30% solids), 70 g of dextrose, 30 g of canola oil, 20 g of salt, 0.06 g of ascorbic acid, 0.625 g of Novamyl® (when present) and 3.75 g of sodium stearoyl lactylate were combined ingredients in a bowl mixer, mixed for 1 minute at low speed, and mixed for 10 minutes at high speed. Three 400 g dough pieces were formed and proofed for 7 minutes. The dough was then rolled to form loaves, placed in bread pans and proofed at 44° C. until they reached a height of 100 mm. The bread loaves were baked at 225° C. for 17 minutes. Crumb hardness (an indicator of staling), resilience and bread volume were measured after baking.

Texture analysis. Analyzing crumb texture was done 5, 8 and 13 days after bread baking. The loafs were cut with an electric knife, using a 2.5 cm gauge. Two slices in the middle of the loaf were analyzed. The evaluation of the crumb hardness and resilience was done with the TA-XT Plus Texture™ Analyzer. The TA-3 probe was used to compress the crumb to a distance of 10 mm (40% compression). Five measurements per slice were made on two slices for a total of 10 measurements. A macro was used for the calculations of the % resilience.

Bake test with Gluzyme® as a control. White pan bread was made without or with addition of commercial (Gluzyme Mono® 10000 BG, dosed at 100 or 200 GOU/kg flour) or yeast cell-associated glucose oxidase (dosed at 127 GOU/kg flour). 1 000 g of white flour, 600 g of water, 40 g of block yeast (˜30% solids), 70 g of dextrose, 30 g of vegetable oil, 20 g of salt, and 0.06 g of ascorbic acid were combined in a bowl mixer, mixed for 1 minute at low speed, and mixed for 9 minutes at high speed. Three 400 g dough pieces were formed and placed in bread pans and proofed at 44° C. until they reached a height of 100 mm. The bread loaves were baked at 225° C. for 17 minutes. Strain M16780 was grown in batch in yeast extract peptone media plus 4% glucose at 32° C. for 24 hours. The cell pellet was obtained by centrifuging whole YPD culture and removing the supernatant. The pellet was assayed as described in the glucose oxidase method description, and a volume equivalent to 127 GOU/kg flour was dosed into dough. Three dough pieces each were proofed to 100 mm height and baked, followed by measurement of oven height. Oven spring (oven height minus proof height) was measured and crumb structure (higher score=finer crumb) was evaluated by visual inspection.

### Example II—Expression of Cell-Associated Maltogenic Alpha-Amylases

The expression of heterologous MAA, especially in the presence of a tether, provided the recombinant yeasts with maltogenic amylase activity both in the cell pellet (FIG. 1A) and, at a larger scale, in the cream yeasts (FIG. 1B). In comparison, the corresponding wild-type strain failed to exhibit any maltogenic amylase activities (FIGS. 1A and 1B). Results shown in FIGS. 1A and 1B were obtained by expressing the heterologous MAA from the promoters of the tdh1 and hor7 genes. Similar results were obtained with a combination of only one promoter (from the hor7 gene; data not shown).

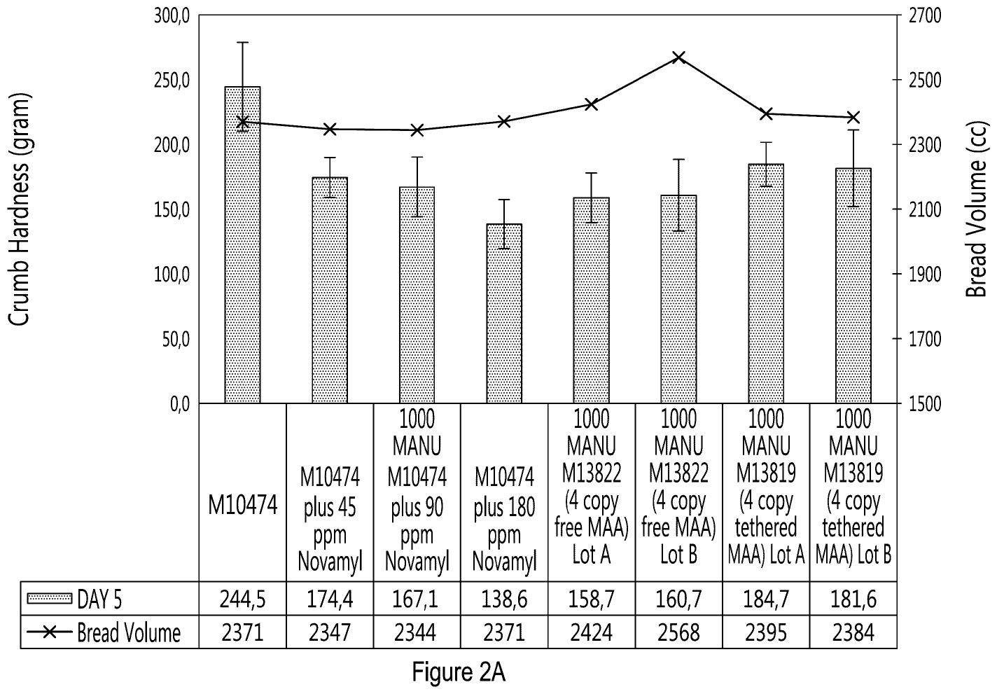

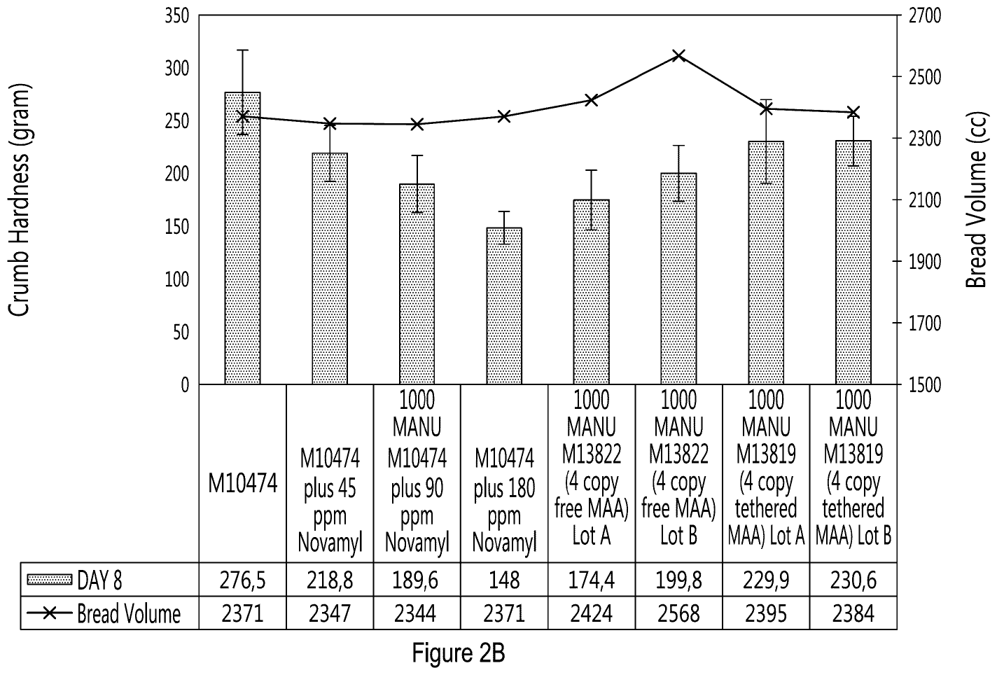

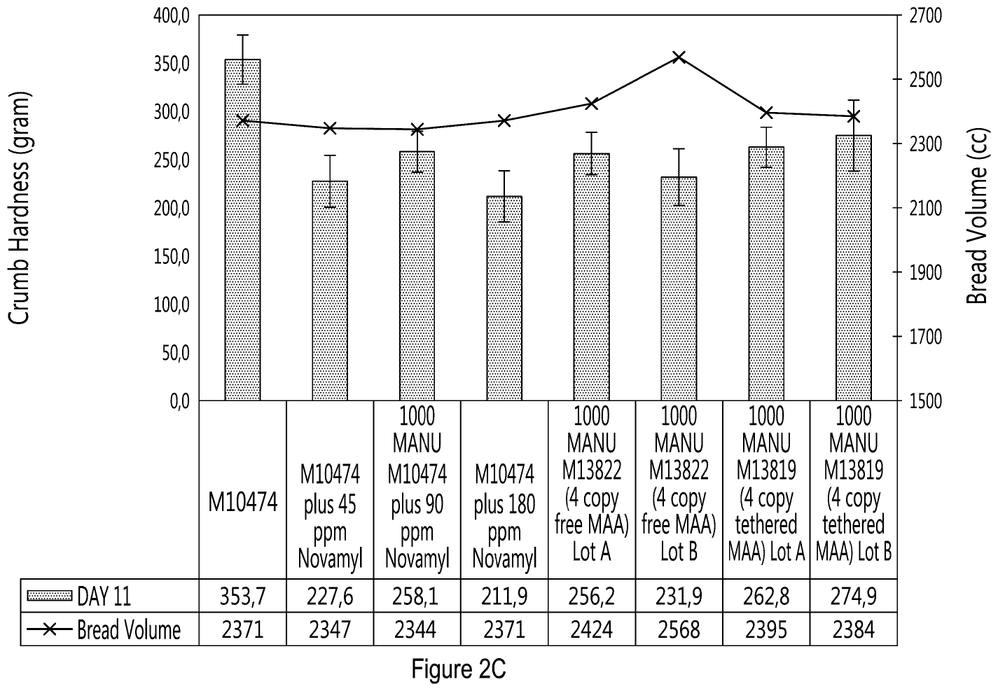

In order to determine the effect(s) of using yeasts expressing heterologous MAA in bread making, different loaf of breads were made with wild-type yeasts (supplemented or not with Novamyl®) or with recombinant yeasts expressing the heterologous MAA. In bread making, quality is associated with softness so the ability to prevent crumb hardness is sought. As shown in FIGS. 2A to 2C, the use of recombinant yeasts expressing the heterologous MAA for making bread reduced crumb hardness, when compared to the bread made with wild-type strain only that is not supplemented with Novamyl®. The use of recombinant yeasts expressing the heterologous MAA provided bread loafs having a similar crumb hardness to those being made with the wild-type yeast supplemented with Novamyl®.

As also shown in FIGS. 2A to 2C, the use of yeasts expressing the heterologous MAA maintained or even increased bread volume, when compared to the wild-type yeasts supplemented or not with Novamyl®.

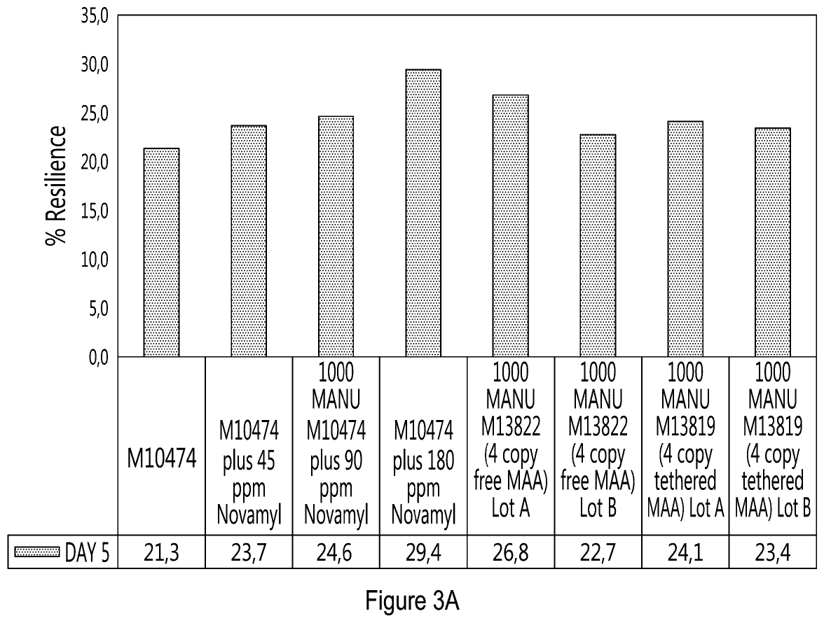

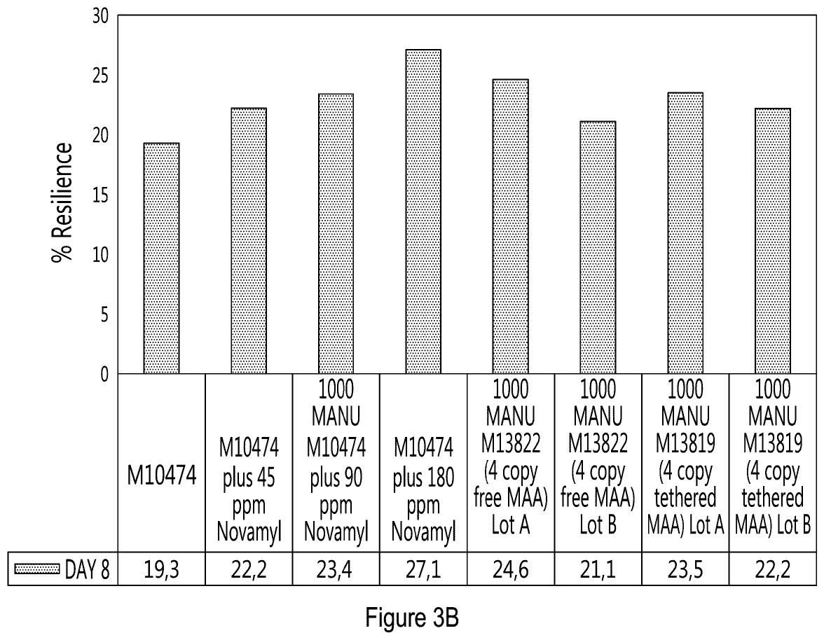

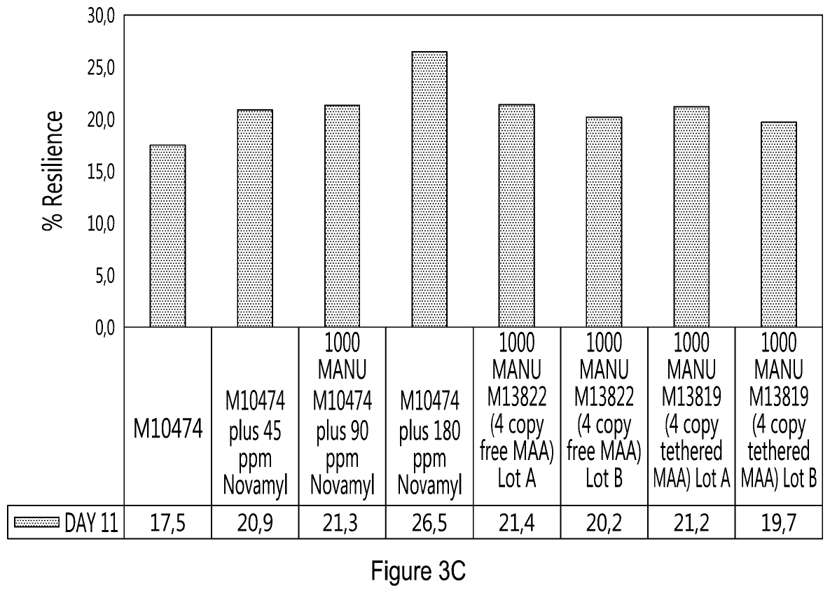

Bread quality can also be assessed by measuring percent resilience, whereas an increase in percent resilience is desirable. As shown in FIGS. 3A to 3C, the use of yeasts expressing the heterologous MAA even increased percent resilience, when compared to the wild-type yeasts which were not supplemented with Novamyl®.

A strain expressing and intracellular G. stearothermophilus MAA (M14851) was propagated (aerobic fed batch on molasses) and MANU activity was determined. As shown in Table 2, in the untreated total broth, between 25.6 and 39.3 MANU activity was detected. After washing and concentrating the cream, between 132 and 288 MANU activity was detected.

TABLE 2
Concentrating yeast biomass concentrates cell-associated maltogenic
amylase. Enzyme activity was determined in Phadebas enzyme
assays with comparison to a dose curve of Novamyl standards
with known maltogenic amylase units (MANU).

Phadebas MANU/ml

| M14851 | Total broth | Washed and concentrated |
|---|---|---|
| propagation | (~6% solids) | cream (19-20% solids) |
| I200617 | 25.6 | 112.2 |
| I210617 | 35.6 | 232.0 |
| I220617 | 39.3 | 287.6 |

Another strain expressing a tethered G. stearothermophilus MAA (M13879) was propagated (aerobic fed batch on molasses) and MANU activity was determined in various yeast preparations. The results are shown Table 3. Cream yeast activity data on 1 day after commercial propagation is the most representative measure of the cream in its original form. All other data were obtained on 8 days after the commercial propagation.

TABLE 3
Phadebas MANU activity per gram dry weight of various
preparations of M13979. Enzyme activity was determined
in Phadebas enzyme assays with comparison to a dose
curve of the enzyme Novamyl ® standards
with known maltogenic amylase units (MANU).

| | Phadebas MANU equivalent/ | |
|---|---|---|
| | gram dry weight | |
| | 1 day after | 8 days after |
| M13979 sample | propagation | propagation |
| Cream | 1087 | 3157 |
| Bead-milled homogenate | | 8698 |
| Cream, spray dried | | 1121 |
| Inactivated cream, spray dried | | 2039 |
| Bead-milled homogenate, spray dried | | 6721 |

MANU and wheat starch activity were determined in different preparations of a yeast strain expressing intracellularly the maltogenic alpha amylase from G. stearothermophilus (M15532) and propagated (aerobic fed batch on molasses). The results are provided in Tables 4 to 8 showing the effects of the different preparations on the level of enzymatic activity observed.

TABLE 4
Phadebas and wheat starch enzyme assays to measure maltogenic
amylase activity on various M15532 preparations. Enzyme
activity was determined in Phadebas enzyme assays with
comparison to a dose curve of the enzyme Novamyl ® standards
with known maltogenic amylase units (MANU).

| | | | Phadebas | Wheat starch |
|---|---|---|---|---|
| M15532 | | | MANU/g | MANU/g |
| propagation | Sample | Form | dry weight | dry weight |
| High protein | Untreated cream | Liquid | 287 | 574 |
| recipe | Bake lab | Liquid | 17826 | 15328 |
| I060917 | autolyzed cream | | | |
| | (pH 7, 48 h, | | | |
| | 55° C.) (liquid) | | | |
| Mix of 4 | Untreated cream | Liquid | 96 | |
| propagations | Bake lab | Liquid | 23614 | 12920 |
| (I280817, | autolyzed cream | | | |
| B300817, | (pH 7, 48 h, | | | |
| B310817, | 55° C.) (liquid) | | | |
| B300817) | Bead-milled | Liquid | 15916 | 12903 |
| | homogenate | | | |
| | (liquid) | | | |
| | Bead-milled | Dry | 10607 | 7764 |
| | homogenate | | | |
| | (spray dried) | | | |

TABLE 5
Activity results in cream, lab-scale autolyzed cream
(incubated 48 h at 55° C., pH 7) and rehydrated
instant dry yeast (IDY) samples. Enzyme activity was
determined in Phadebas enzyme assays with comparison
to a dose curve of the enzyme Novamyl ® standards
with known maltogenic amylase units (MANU).

| | | Phadebas | Phadebas |
|---|---|---|---|
| | % | MANU/ml | MANU/g |
| M15532 Sample | solids | of sample | DCW |
| Cream | 17.9 | 58 | 325 |
| Cream after 48 h, 55° C., pH 7 | 17.9 | 3572 | 19955 |
| 37° C. rehydrated IDY | 15.9 | 545 | 3438 |
| Cold shocked IDY | 15.4 | 454 | 2958 |

TABLE 6
Dry weight and enzyme activity balances in autolysate, yeast extract, ultrafiltration retentate and yeast cell wall preparations
before and after drying of different preparations of the yeast strain M15532. Enzyme activity was determined in Phadebas enzyme
assays with comparison to a dose curve of the enzyme Novamyl ® standards with known maltogenic amylase units (MANU).

| | | | | | | | | | | MANU | |
|---|---|---|---|---|---|---|---|---|---|---|---|
| | | | | | | | | | | BALANCE | |
| | BEFORE | | AFTER DRYING | | | | | | | PROCESS + | |
| | DRYING | | | | BAKE LAB | | NRC24NOV | | | DRYING | |
| | dw | MAA | dw | NRC10NOV | MANU/gdw | | MANU/gdw | | dwt | BAKE | |
| SAMPLE | % | MANU/gdw | % | MANU/gdw | AV | % RD | AV | % CV | BALANCE | LAB | NRC24NOV |
| AUTOLYSATE | 18.0 | 8983 | 91.3 | 13568 | 14812 | 6% | 16864 | 17% | 100 | 100 | 100 |
| YE | 11.6 | 14422 | 93.7 | 23067 | 27566 | 5% | 24028 | 14% | 47 | 87 | 66 |
| 10 kDa | 15.1 | 83332 | 93.7 | 25984 | 80817 | 5% | 75552 | 15% | 18 | 98 | 80 |
| RETENTATE | | | | | | | | | | | |
| CW | 37.0 | 883 | 94.1 | 700 | 3757 | 4% | 3421 | 4% | 53 | 14 | 11 |

TABLE 7
Results of separation of yeast extract from total autolysate
and enzyme recovery with and without washing of yeast strain
M15532. YE (yeast extract) separation yield is the recovery
of solids from separation only (WF = 0) and of separation
plus one or two washes (WF = 1 or 2, respectively), relative
to the starting solids in the autolysate. YE MANU recovery is
the activity (in Phadebas MANU) from separation only (WF =
0) and of separation plus one or two washes (WF = 1 or 2),
relative to starting total Phadebas MANU in the autolysate.
Enzyme activity was determined in Phadebas enzyme assays with
comparison to a dose curve of the enzyme Novamyl ®
standards with known maltogenic amylase units (MANU).

| WASH | YE | YE MANU | |
|---|---|---|---|
| FACTOR | SEPARATION | RECOVERY | % DW |
| (WF) | YIELD (%) | (%) | in YE |
| 0 | 36 | 58 | 12.3 |
| 1 | 50 | 71 | 8.3 |
| 2 | 54 | 75 | 6.0 |

TABLE 8
Results of ultrafiltration of yeast extract of M15532 with
a 10 kDa molecular weight cutoff. YE is yeast extract, obtained
by centrifuging fermenter autolysate in 1 liter bottles for
10 minutes at 11,000 RCF, to mimic separation at industrial
scale. Retentate is the sample retained by ultrafiltration
and permeate is the sample not retained. Phadebas MANU/ml
was determined for each samples and MANU/g DW (dry weight)
was calculated based on the dry weight per sample. Enzyme
activity was determined in Phadebas enzyme assays with comparison
to a dose curve of the enzyme Novamyl ® standards
with known maltogenic amylase units (MANU).

| | | % | | | | DW |
|---|---|---|---|---|---|---|
| | Concen- | DW | | | MANU | bal- |
| | tration | in | | MANU/g | balance | ance |
| Sample | factor | sample | MANU/mL | DW | (%) | (%) |
| YE | 1.0 | 11.6 | 1672 | 14422 | 100 | 100 |
| 10 kDa | 3.5 | 15.5 | 12583 | 83332 | 222 | 38 |
| RETEN- | | | | | | |
| TATE | | | | | | |
| 10 kDa | | 10.9 | 22 | 203 | 1 | 67 |
| PER- | | | | | | |
| MEATE | | | | | | |

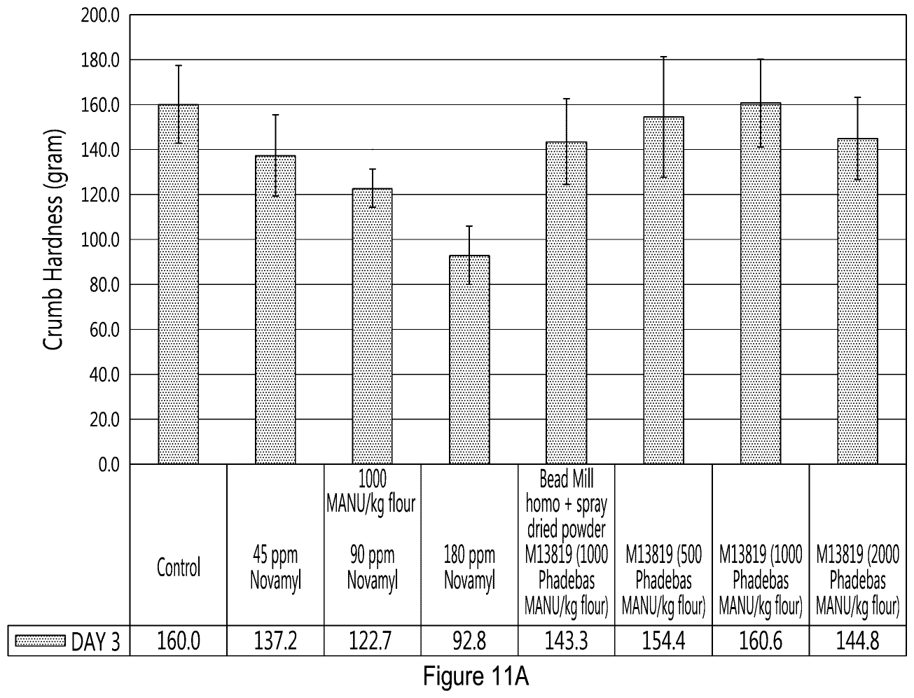

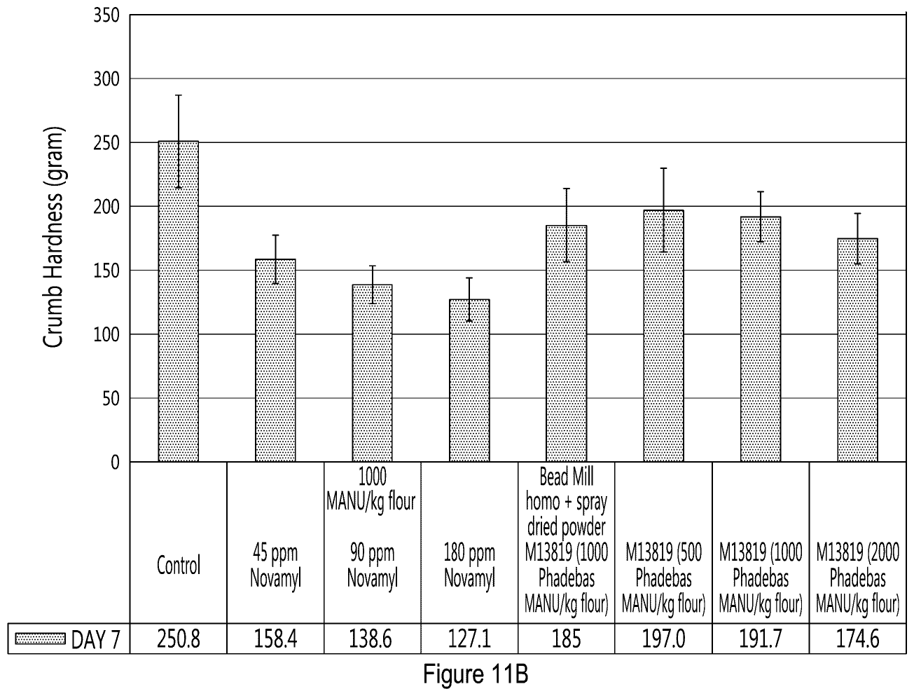

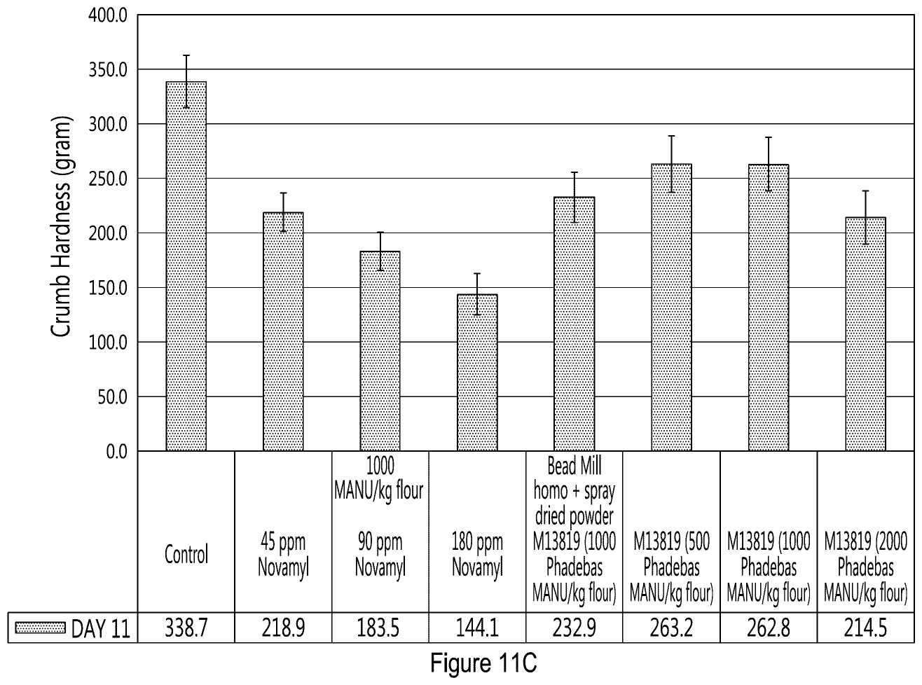

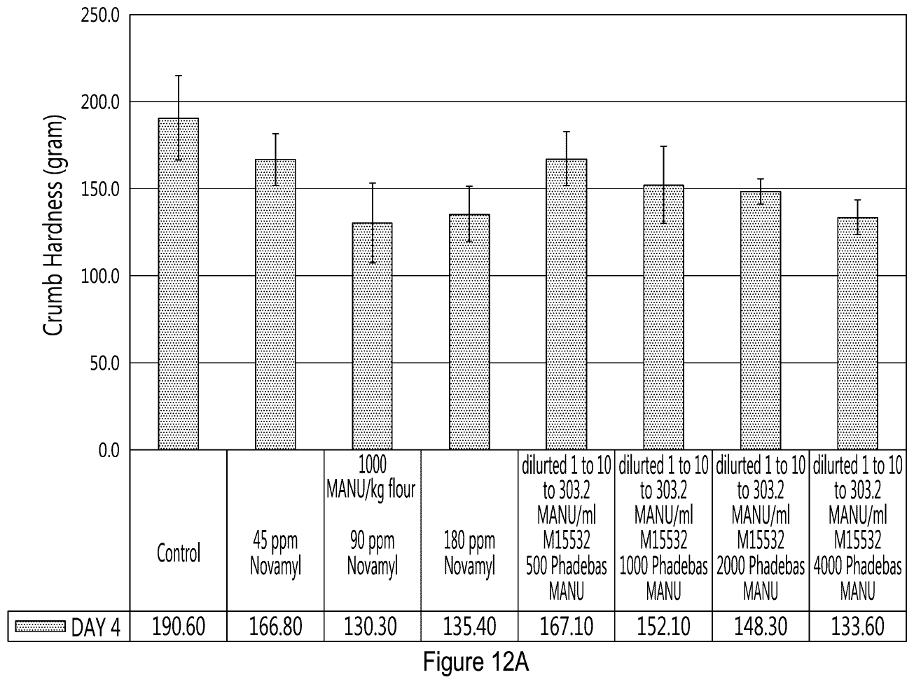

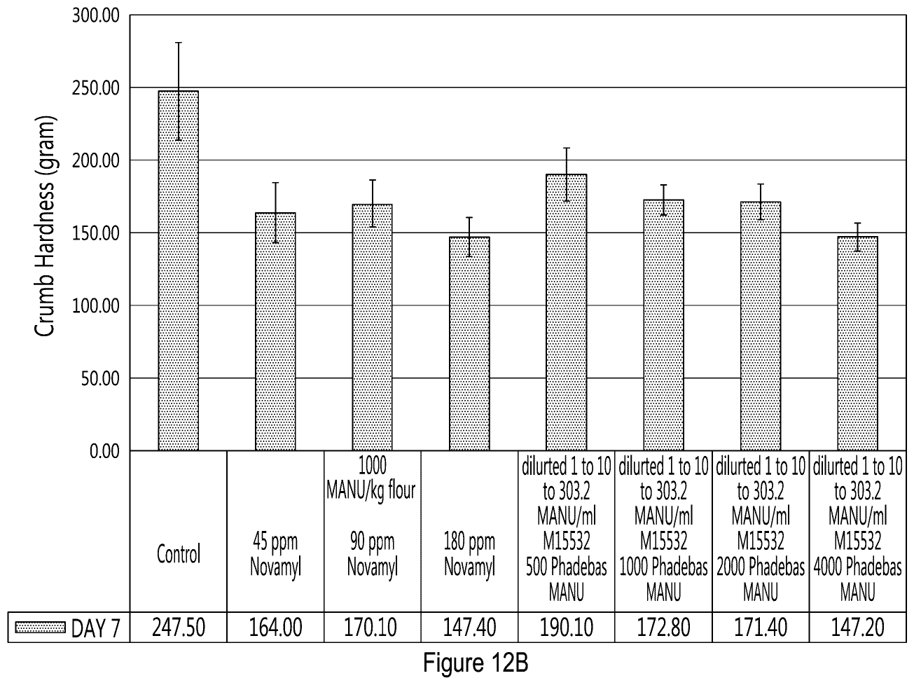

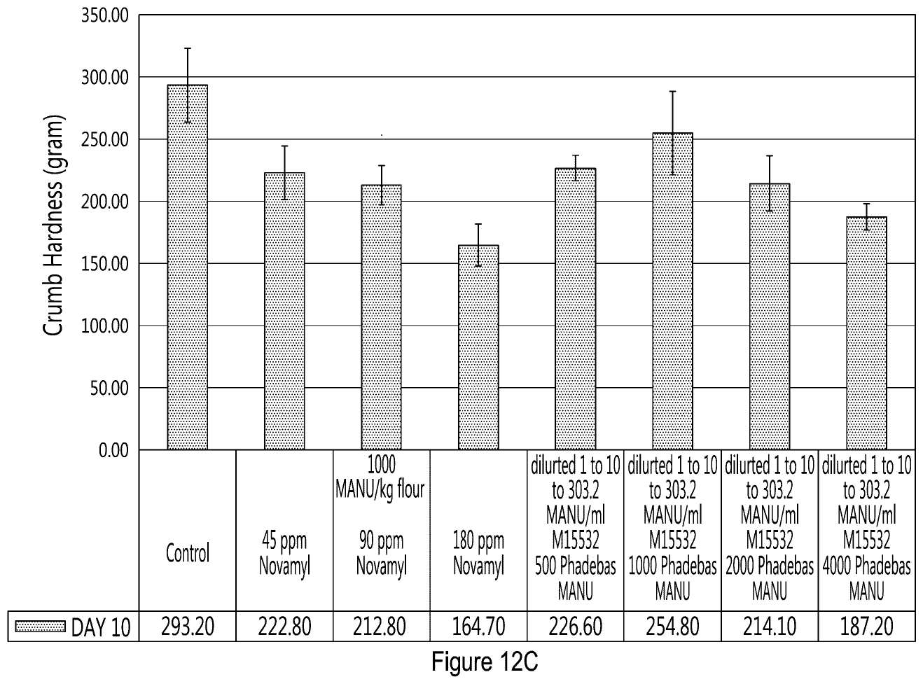

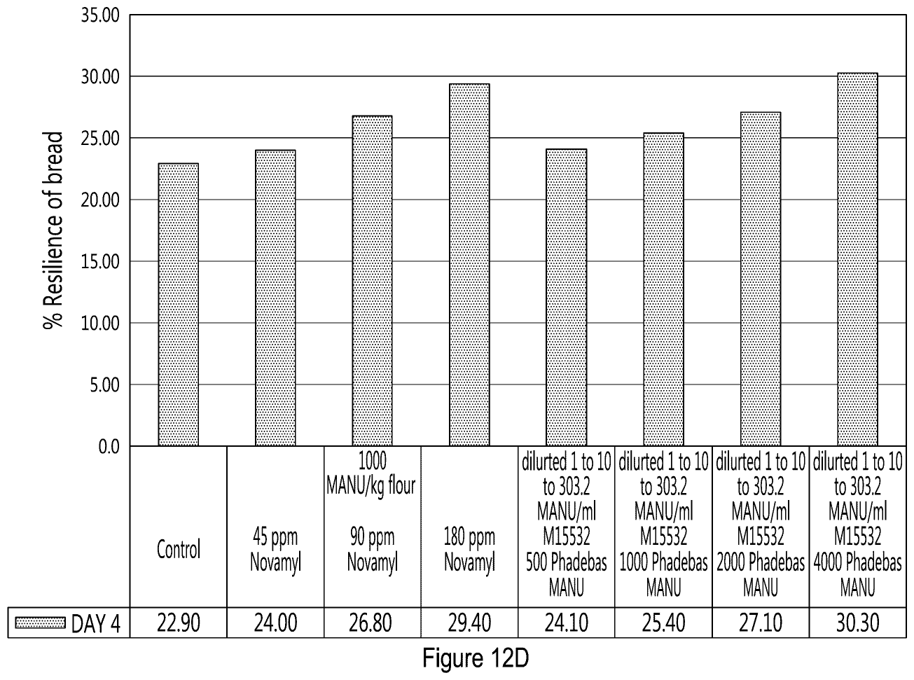

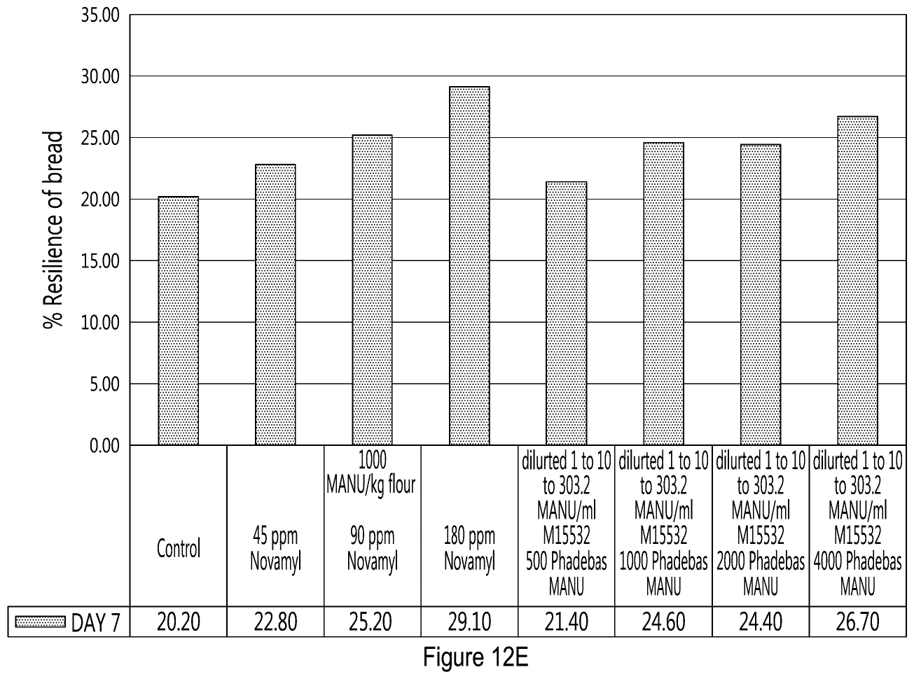

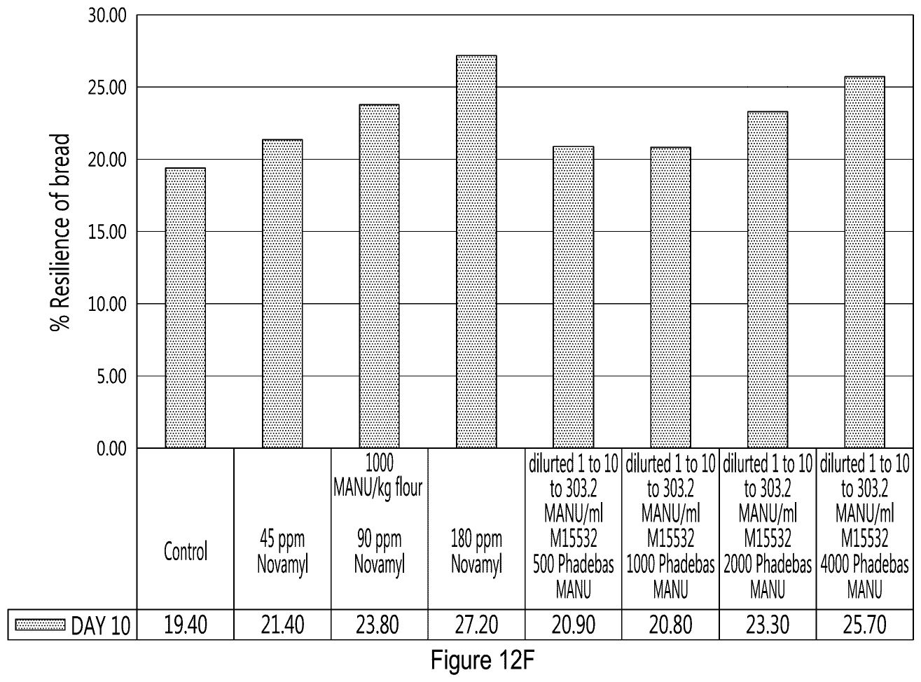

Different preparations (e.g., cream and spray-dried) yeast strains M13979 (expressing a tethered MAA) and M15531 (expressing an intracellular MAA) have been used to make bread loafs. The use of yeasts strains M13979 and M15531, when compared to control breads made in the absence of a dough conditioner, reduces the bread's crumb hardness (FIGS. 11 and 12A) while maintaining its volume (FIG. 11) and increasing its resilience (FIG. 12B).

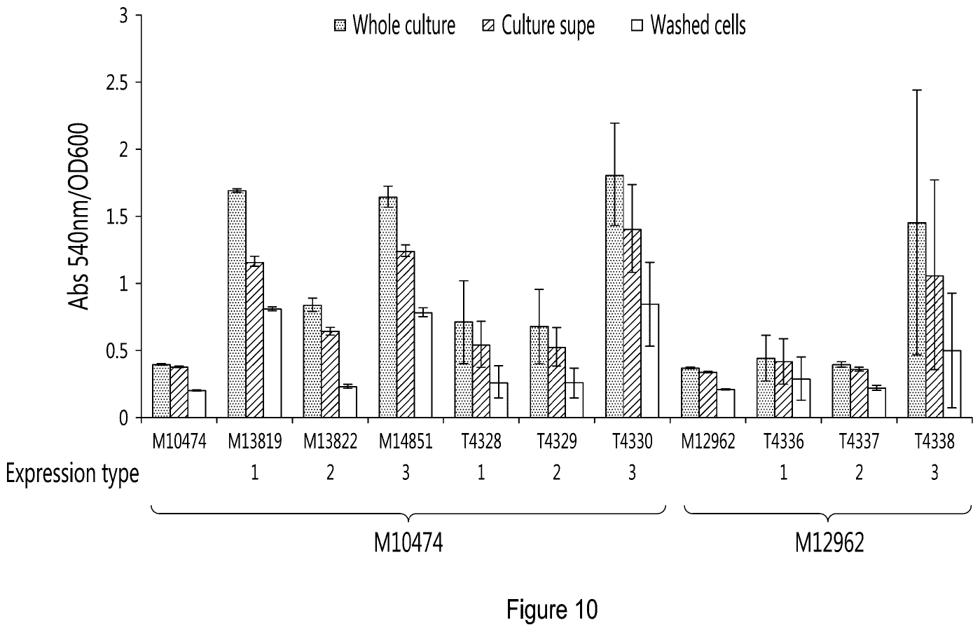

The wheat starch activity normalized to cell density was determined in the whole culture, the culture supernatant and the washed cells of various yeast strains expressing the maltogenic alpha amylase from G. stearothermophilus expressed in a secreted form, in a tethered form or expressed intracellularly as explained in the legend of FIG. 10. The results are shown in FIG. 8 and indicated that the highest activities are observed when the MAA is expressed intracellularly.

### Example III—Expression of Heterologous Alpha-Amylases, Glucoamylases, Phytases, Glucose Oxidases and Fungal Amylases

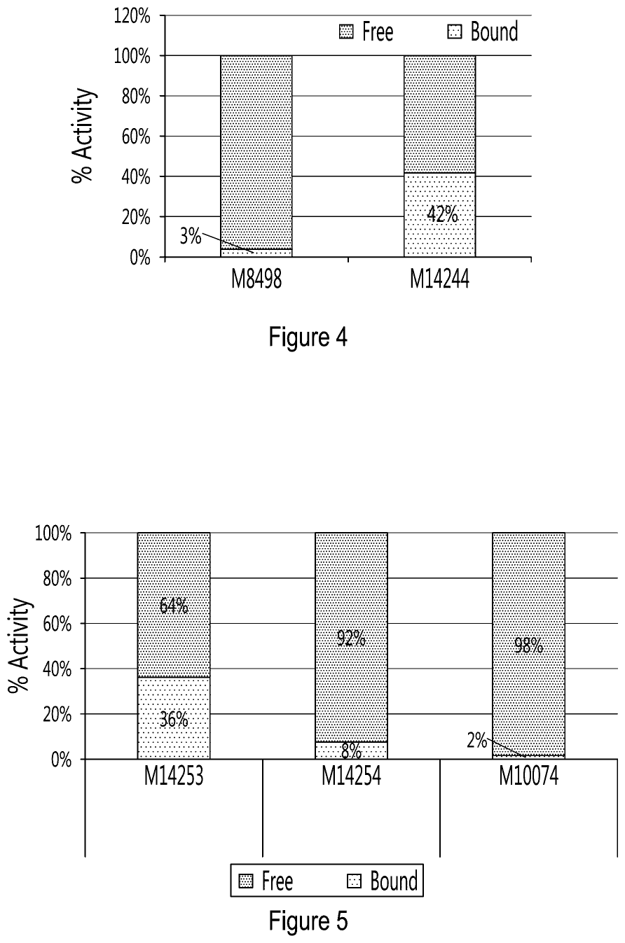

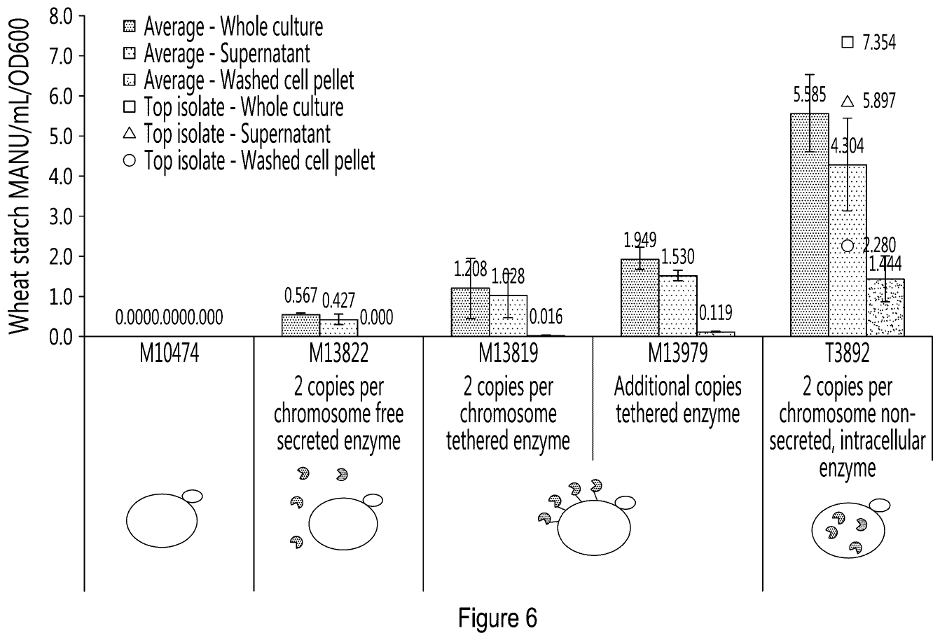

An heterologous glucoamylase (GA) was expressed in S. cerevisiae from the promoter of the tef2 gene. When GA was expressed as a tethered enzyme, activity associated with cellular pellet is increased (FIG. 4).

An heterologous alpha-amylase (AA) from the promoter of the tef2 gene. When the AA was expressed as a tethered enzyme, activity associated with pellet is increased, especially in the presence of a linker (FIG. 5).

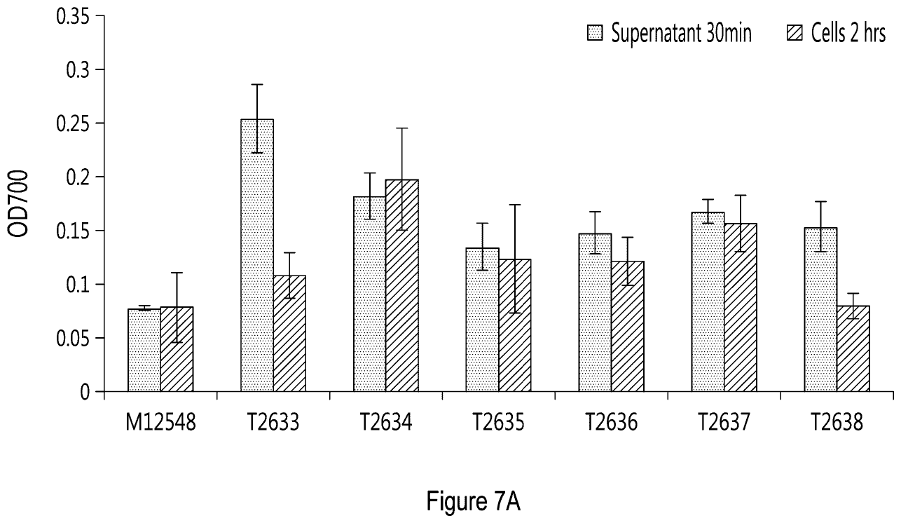

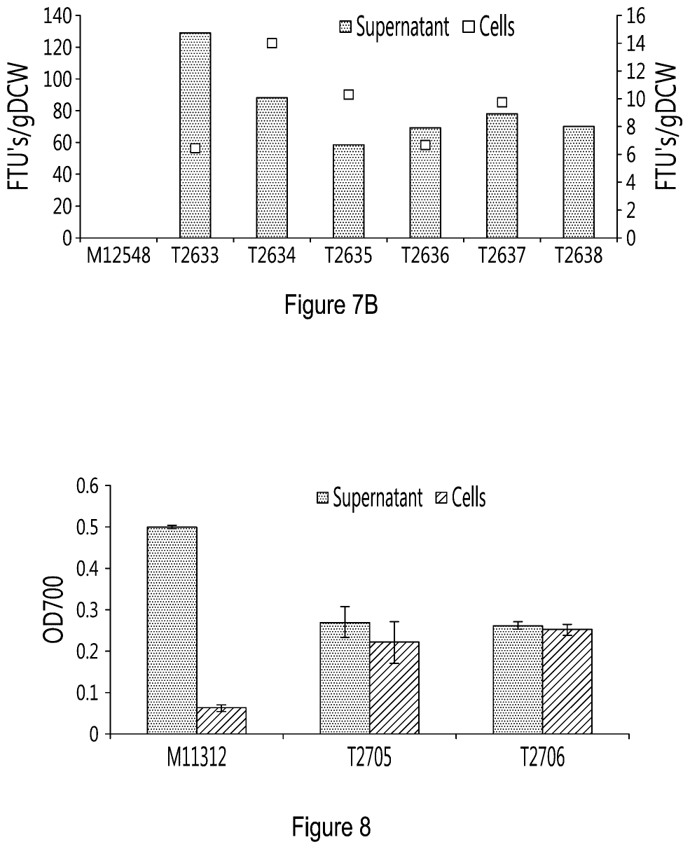

Various preparations of yeast strains expressing the phytase from C. braakii were made and their FTU activity was determined. Some strains expressed the phytase in a secreted form (T2633), other strains expressed the phytase in a tethered form (T2634, T2635, T2636, T2637 and T2638) using different tethers. The results are shown in FIGS. 7A and 7B for both the supernatant and the cells themselves.

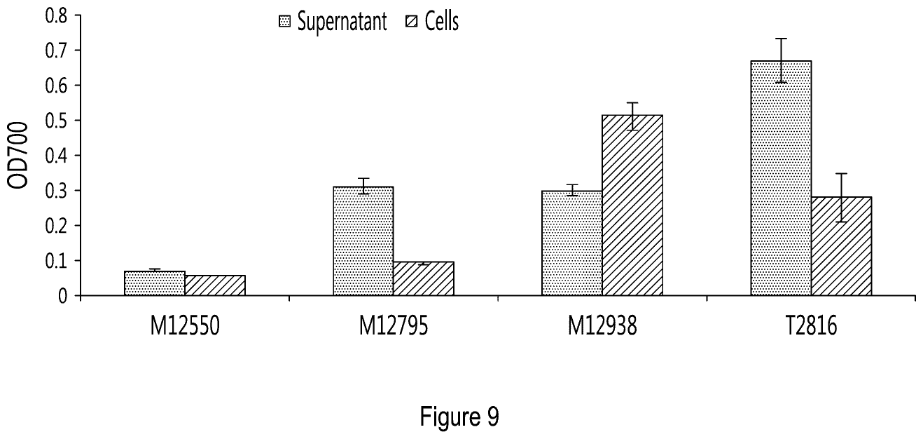

Various preparations of yeast strains expressing the phytase from E. coli were made and their FTU activity was determined. Some strains expressed the phytase in a secreted form (M11312), other strains expressed the phytase in a tethered form (T2705, T2706, M12795, M12938, T2816) using the different configurations of tethers. The results are shown in FIGS. 8 and 9 for both the supernatant and the cells themselves.

Heterologous chimeric thermo-tolerant P. furiosus alpha-amylase-SPI1 constructs and T. hydrothermalis alpha-amylase-CCW12 constructs were made using various truncations of the tethering moieties. The alpha-amylase activity associated with the washed cells of the strains expressing the chimeric polypeptides with the truncated GPI anchoring portions were compared to the non-truncated GPI anchoring portion is shown in FIGS. 13 and 14.

As seen from FIG. 13, the chimeric polypeptide with the full length tethering moiety (expressed from strain M15222) showed the same or higher alpha-amylase activity than the polypeptides with truncated tethering moieties (expressed from strains M15774 (21 aa-long truncation), M15771 (51 aa-long truncation), M1577 (81 aa-long truncation) or M15772 (130 aa-long truncation)).

As seen from FIG. 14, the chimeric polypeptides with the full length tethering moiety (expression from strain M15215) exhibited similar or higher alpha-amylase activity when compared to chimeric polypeptides having a truncated tethering moiety (expressed from strains M15773 (24 aa-long truncation), M15776 (49 aa-long truncation), M16251 (74 aa-long truncation) or M15775 (99 aa-long truncation)).

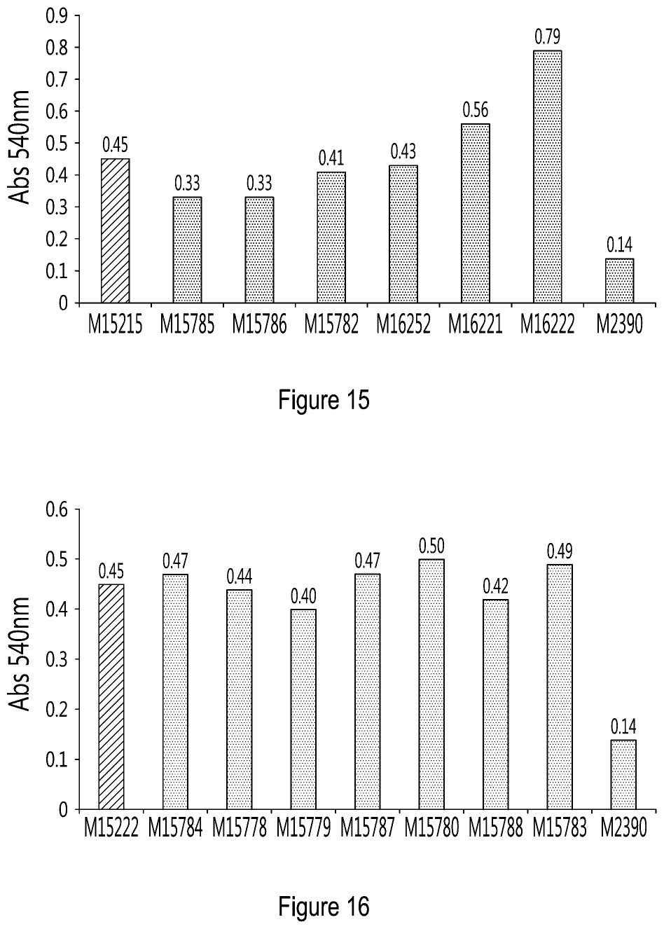

Heterologous chimeric thermo-tolerant P. furiosus alpha-amylase-SPI1 constructs and T. hydrothermalis alpha-amylase-CCW12 constructs were made using various linkers and the same tethering moiety. The alpha-amylase activity associated with the washed cells of the strains expressing the chimeric polypeptides with the different linkers is shown in FIGS. 15 and 16.

As seen from FIG. 15, the alpha-amylase activity of all the strains was higher than the control strain (M2390), irrespective of type of linker used. The alpha-amylase activity was the highest when linker 7 (SEQ ID NO: 99) was used (strain M16222).

As seen from FIG. 16, the alpha-amylase activity of all the strains was higher than the control strain (M2390), irrespective of type of linker used. The alpha-amylase activity was the highest when linker 5 (SEQ ID NO: 97) was used (strain M15780).

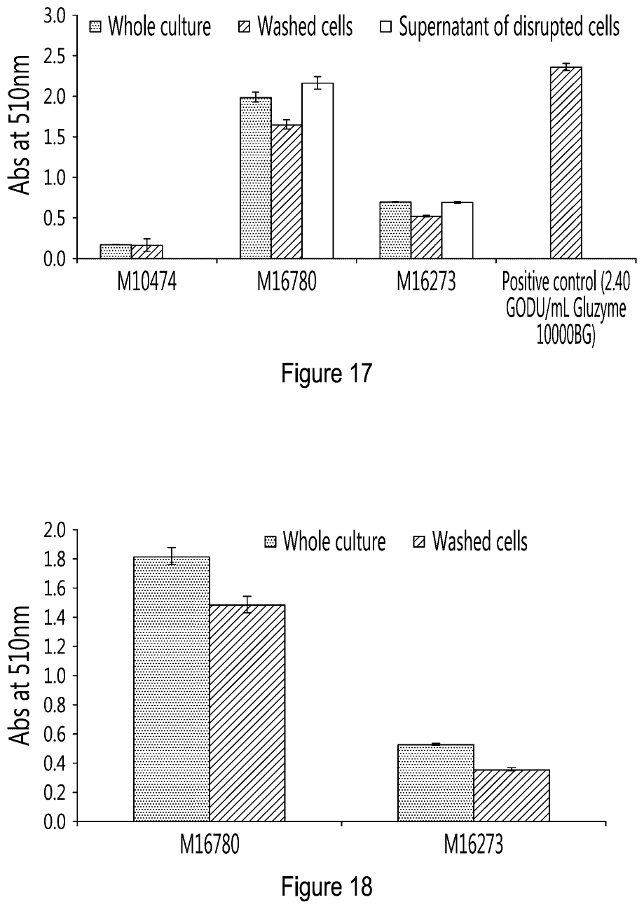

Heterologous chimeric glucose oxidase (GO) constructs were expressed intracellularly or in a secreted form. The GO activity obtained from various cellular fractions was compared to a control strain (M10474) or a positive control enzymatic preparation Gluzyme Mono® (FIG. 17). The GO activity associated with strains M16780 and M16273 was higher than the control GO activity associated with the parental strain M10474 (FIG. 18).

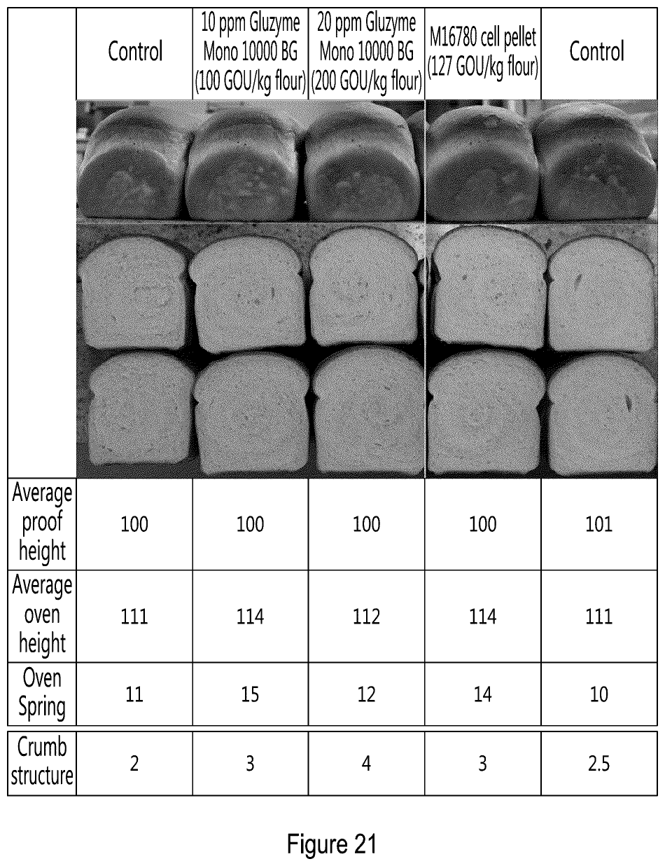

Strain M16780 was also used to supplement the dough of bread loaves which were compared to negative control (non-supplemented dough) loaves and positive control (Gluzyme Mono® supplemented dough) loaves. As shown in FIG. 21, higher oven spring and finer crumb structure (which is observed for M16780 cell pellet doughs) are indicators of glucose oxidase function in the supplemented dough.

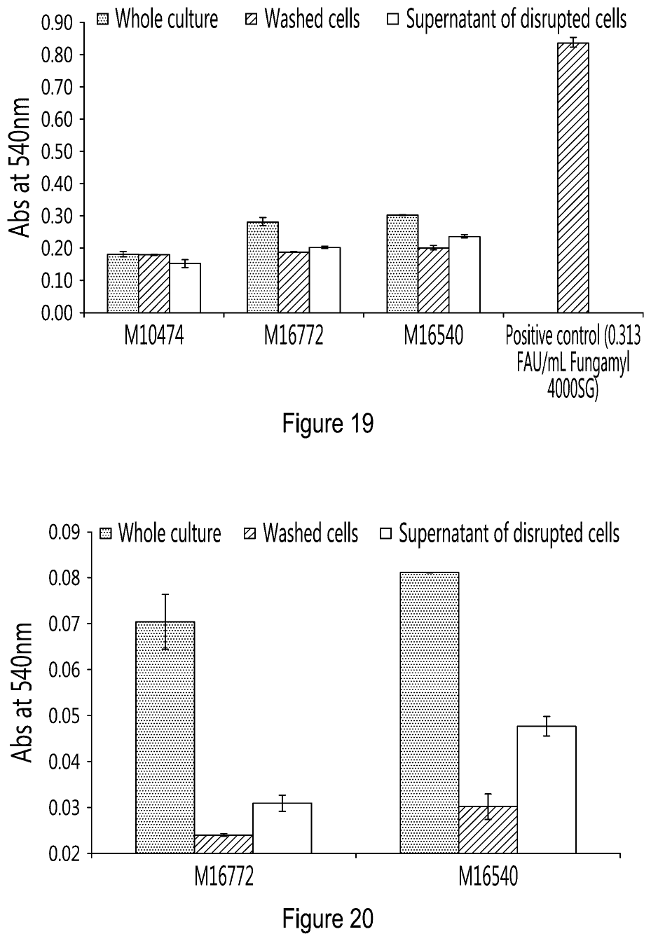

Heterologous chimeric fungal amylase (FA) constructs were expressed in a secreted form. The FA activity obtained from various cellular fractions was compared to control strain M10474 or a positive control enzymatic preparation Fungamyl® (FIG. 19). The FA activity associated with strains M16772 and M16540 was higher than the control activity associated with the parental strain M10474 (FIG. 20).

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Pérez-Torrado R, Bruno-Bárcena J M, Matallana E. Monitoring stress-related genes during the process of biomass propagation of Saccharomyces cerevisiae strains used for wine making. Appl Environ Microbiol. 2005 November;71(11):6831-7.
Praekelt U M, Meacock P A. MOL1, a Saccharomyces cerevisiae gene that is highly expressed in early stationary phase during growth on molasses. Yeast. 1992 September;8(9):699-710.

What is claimed is:

1. A process for making a food or a feed product, said process comprising including a recombinant yeast host cell or an additive comprising the recombinant yeast host cell in the food or the feed product, wherein the recombinant yeast host cell has a heterologous nucleic acid molecule encoding a cell-associated heterologous glucose oxidase, wherein the heterologous nucleic acid molecule is operatively associated with a heterologous promoter allowing the expression of the heterologous nucleic acid molecule during propagation under aerobic conditions.

2. The process of claim 1, wherein the heterologous nucleic acid molecule allows intracellular expression of the heterologous glucose oxidase.

3. The process of claim 1, wherein the heterologous nucleic acid molecule allows the expression of a membrane-associated glucose oxidase.

4. The process of claim 1, wherein the heterologous nucleic acid molecule allows the expression of a tethered heterologous glucose oxidase.

5. The process of claim 4, wherein the tethered heterologous glucose oxidase is a chimeric protein of formula (I) or (II):
$$( {NH}_{2} ) FFE - L - TT ( COOH ) \qquad ( I )$$
$$( {NH}_{2} ) TT - L - FFE ( COOH ) \qquad ( II )$$
wherein FFE is the glucose oxidase;
L is present or absent and is an amino acid linker;
TT is an amino acid tethering moiety for associating the glucose oxidase to a cell wall of the recombinant yeast host cell;
(NH₂) indicates the amino terminus of the chimeric protein;
(COOH) indicates the carboxyl terminus of the chimeric protein; and
“-” is an amide linkage.

6. The process of claim 1, wherein the heterologous promoter is a native or a heterologous promoter.

7. The process of claim 6, wherein the heterologous promoter comprises the promoter from the tdh1 gene, the hor7 gene, the hsp150 gene, the hxt7 gene, the gpm1 gene, the pgk1 gene and/or the stl1 gene.

8. The process of claim 3, wherein the membrane-associated heterologous glucose oxidase has a native or a heterologous signal peptide.

9. The process of claim 4, wherein the tethered heterologous glucose oxidase has a native or a heterologous signal peptide.

10. The process of claim 1 being from the genus Saccharomyces sp.

11. The process of claim 1 being from the species Saccharomyces cerevisiae.

12. The process of claim 1, further comprising adding the recombinant yeast host cell or the additive to a flour, a dough or a batter.

13. The process of claim 1, wherein the additive is a food additive, a dough conditioner, or a feed additive.

14. The process of claim 1, wherein the additive is a live yeast composition or an inactivated yeast composition.

15. The process of claim 1, further comprising fermenting the food or the feed product in the presence of the recombinant yeast host cell.

16. The process of claim 1, further comprising baking the food or the feed product to provide a baked product.

17. The process 16, wherein the baked product is a bread.


SEQUENCE LISTING (108 sequences)

SEQ ID NO: 1 - protein, 719 aa, Geobacillus stearothermophilus
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 2 - protein, 499 aa, Aspergillus oryzae
MMVAWWSLFLYGLQVAAPALAATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 3 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 4 - protein, 551 aa, Pseudomonas saccharophila
MSHILRAAVLAAVLLPFPALADQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 5 - protein, 926 aa, Bacillus naganoensis
DGNTTNIVVHYFRPSGDYTDWNLWMWPENGDGAEYDFNQPTDSYGEVASVDIPGNPSQVGIIVRKGNWDAKDIDSDRYIDLSKGHEIWLVQGNSQIFYSEKDAEAAAQPAVSNAYLDASNQVLVKLSQPFTLGEGSSGFTVHDDTANKDIPVTSVSDANQVTAVLAGTFQHIFGGSDWAPDNHNTLLKKVNSNLYQFSGNLPEGNYQYKVALNDSWNNPSYPSDNINLTVPAGGAHVTFSYIPSTHAVYDTINNPNADLQVDSSGVKTDLVAVTLGENPDVSHTLSIQTEDYQAGQVIPRKVLDSSQYYYSGDDLGNTYTKNATTFKVWAPTSTQVNVLLYNSATGAVTKTVPMTASGHGVWEATVNQDLENWYYMYEVTGQGSTRTAVDPYATAIAPNGTRGMIVDLAKTDPAGWESDKHITPKNIEDEVIYEMDVRDFSIDSNSGMKNKGKYLALTEKGTKGPDNVKTGVDSLKQLGITHVQLQPVFAFNSVNENDPTQYNWGYDPRNYNVPEGQYATNANGTTRIKEFKEMVLSLHQDHIGVNMDVVYNHTFATQISDFDKIVPEYYYRTDDAGNYTNGSGTGNEIAAERPMVQKFIIDSLKFWVNEYHVDGFRFDLMALLGKDTMSKAATQLHAIDPGIALYGEPWTGGTSALPADQLLTKGAQKGMGVAVFNDNLRNGLDGSVFDSSAQGFATGATGLTDAIKNGVEGSINDFTASPGETINYVTSHDNYTLWDKIAQSNPNDSEADRIKMDELAQAIVMTSQGIPFMQGGEEMLRTKGGNDNSYNAGDVVNEFDWSRKAQYPDVFNYYSGLIHLRLDHPAFRMTTANEINSHLQFLNSPENTVAYELSDHANKDTWGNIVVIYNPNKTAETINLPSGKWEINATSGKVGESTLGQAEGSVQVPGISMMILHQEVSPSDGK

SEQ ID NO: 6 - protein, 829 aa, Bacillus acidopullulyticus
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFNVGVSDFDKIVPQYYYRTDSAGNYTNGSGVGNEIATERPMVQKFVLDSVKYWVKEYHIDGFRFDLMALLGKDTMAKISKELHAINPGIVLYGEPWTGGTSGLSSDQLVTKGQQKGLGIGVFNDNIRNGLDGNVFDKSAQGFATGDPNQVNVIKNRVMGSISDFTSAPSETINYVTSHDNMTLWDKISASNPNDTQADRIKMDELAQAVVFTSQGVPFMQGGEEMLRTKGGNDNSYNAGDSVNQFDWSRKAQFENVFDYYSWLIHLRDNHPAFRMTTADQIKQNLTFLDSPTNTVAFELKNHANHDKWKNIIVMYNPNKTAQTLTLPSGNWTIVGLGNQVGEKSLGHVNGTVEVPALSTIILHQGTSEDVIDQN

SEQ ID NO: 7 - protein, 776 aa, Pseudomonas amyloderamosa
MKCPKILAALLGCAVLAGVPAMPAHAAINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISK

SEQ ID NO: 8 - protein, 500 aa, Thermus thermophilus
MELPRAFGLLLHPTSLPGPYGVGVLGQEARDFLRFLKEAGGRYWQVLPLGPTGYGDSPYQSFSAFAGNPYLIDLRPLAERGYVRLEDPGFPQGRVDYGLLYAWKWPALKEAFRGFKEKASPEEREAFAAFREREAWWLEDYALFMALKGAHGGLPWNRWPLPLRKREEKALREAKSALAEEVAFHAFTQWLFFRQWGALKAEAEALGIRIIGDMPIFVAEDSAEVWAHPEWFHLDEEGRPTVVAGVPPDYFSETGQRWGNPLYRWDVLEREGFSFWIRRLEKALELFHLVRIDHFRGFEAYWEIPASCPTAVEGRWVKAPGEKLFQKIQEVFGEVPVLAEDLGVITPEVEALRDRFGLPGMKVLQFAFDDGMENPFLPHNYPAHGRVVVYTGTHDNDTTLGWYRTATPHEKAFMARYLADWGITFREEEEVPWALMHLGMKSVARLAVYPVQDVLALGSEARMNYPGRPSGNWAWRLLPGELSPEHGARLRAMAEATERL

SEQ ID NO: 9 - DNA, 1641 bp, synthetic construct
  misc_feature 1..1641: note = Flo1 tether
  source 1..1641: mol_type = other DNA
  CDS 1..1641
aaggacaatagctcgacgattgaaggtagatacccatacgacgttccagactacgctctgcaggctagtggtggtggtggttctggtggtggtggttctggtggtggtggttctgctagcatcagaactccaaccagtgaaggtttggttacaaccaccactgaaccatggactggtacttttacttcgacttccactgaaatgtctactgtcactggaaccaatggcttgccaactgatgaaactgtcattgttgtcaaaactccaactactgccatctcatccagtttgtcatcatcatcttcaggacaaatcaccagctctatcacgtcttcgcgtccaattattaccccattctatcctagcaatggaacttctgtgatttcttcctcagtaatttcttcctcagtcacttcttctctattcacttcttctccagtcatttcttcctcagtcatttcttcttctacaacaacctccacttctatattttctgaatcatctaaatcatccgtcattccaaccagtagttccacctctggttcttctgagagcgaaacgagttcagctggttctgtctcttcttcctcttttatctcttctgaatcatcaaaatctcctacatattcttcttcatcattaccacttgttaccagtgcgacaacaagccaggaaactgcttcttcattaccacctgctaccactacaaaaacgagcgaacaaaccactttggttaccgtgacatcctgcgagtctcatgtgtgcactgaatccatctcccctgcgattgtttccacagctactgttactgttagcggcgtcacaacagagtataccacatggtgccctatttctactacagagacaacaaagcaaaccaaagggacaacagagcaaaccacagaaacaacaaaacaaaccacggtagttacaatttcttcttgtgaatctgacgtatgctctaagactgcttctccagccattgtatctacaagcactgctactattaacggcgttactacagaatacacaacatggtgtcctatttccaccacagaatcgaggcaacaaacaacgctagttactgttacttcctgcgaatctggtgtgtgttccgaaactgcttcacctgccattgtttcgacggccacggctactgtgaatgatgttgttacggtctatcctacatggaggccacagactgcgaatgaagagtctgtcagctctaaaatgaacagtgctaccggtgagacaacaaccaatactttagctgctgaaacgactaccaatactgtagctgctgagacgattaccaatactggagctgctgagacgaaaacagtagtcacctcttcgctttcaagatctaatcacgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagcagcatggtaggatatagtacagcttctttagaaatttcaacgtatgctggcagtgccaacagcttactggccggtagtggtttaagtgtcttcattgcgtccttattgctggcaattatttaa

SEQ ID NO: 10 - protein, 546 aa, synthetic construct
  REGION 1..546: note = Synthetic Construct
KDNSSTIEGRYPYDVPDYALQASGGGGSGGGGSGGGGSASIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 11 - DNA, 807 bp, synthetic construct
  misc_feature 1..807: note = Sed1 tether
  source 1..807: mol_type = other DNA
  CDS 1..807
aaggacaatagctcgacgattgaaggtagatacccatacgacgttccagactacgctctgcaggctagtggtggtggtggttctggtggtggtggttctggtggtggtggttctgctagcgctcttccaactaacggtacttctactgaagctccaactgatactactactgaagctccaaccaccggtcttccaaccaacggtaccacttcagctttcccaccaactacatctttgccaccaagcaacactaccaccactcctccttacaacccatctactgactacaccactgactacactgtagtcactgaatatactacttactgtccagaaccaaccactttcaccacaaacggtaagacttacaccgtcactgaaccaaccacattgactatcactgactgtccatgcaccattgaaaagccaacaaccacatcaaccaccgaatacactgtagtcactgagtacactacttactgtccagaaccaaccactttcaccacaaacggtaagacttacaccgtcactgaaccaaccactttgactatcactgactgtccatgtactattgaaaagagcgaagcccctgagtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattctaa

SEQ ID NO: 12 - protein, 268 aa, synthetic construct
  REGION 1..268: note = Synthetic Construct
KDNSSTIEGRYPYDVPDYALQASGGGGSGGGGSGGGGSASALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLF

SEQ ID NO: 13 - DNA, 759 bp, synthetic construct
  misc_feature 1..759: note = Tir1 tether
  source 1..759: mol_type = other DNA
  CDS 1..759
aaggacaatagctcgacgattgaaggtagatacccatacgacgttccagactacgctctgcaggctagtggtggtggtggttctggtggtggtggttctggtggtggtggttctgctagcagcttagcttctgattcttcctctggattttccttaagcagtatgccagctggtgttttggatatcggtatggctttagcttccgccactgacgactcctacactactttgtactctgaggttgactttgctggtgttagcaagatgttgaccatggttccatggtactcctctagattggaaccagctttgaagtctttgaatggtgatgcttcttcttctgctgccccaagctcttctgctgctccaacttcttctgctgccccaagctcatctgctgccccaacttcttctgctgcctcaagctcttctgaagctaagtcttcttctgctgccccaagctcttctgaagctaagtcttcttctgctgccccaagctcttctgaagctaagtcttcttctgctgccccaagctcttctgaagctaagtcttcttctgctgctccaagctccactgaagctaagataacttctgctgctccaagctccactggtgccaagacctctgccatctctcaaattaccgatggtcaaatccaagctaccaaggctgtttctgagcaaactgaaaacggtgctgctaaggcctttgttggtatgggtgctggtgttgtcgcagctgccgctatgttgttataa

SEQ ID NO: 14 - protein, 252 aa, synthetic construct
  REGION 1..252: note = Synthetic Construct
KDNSSTIEGRYPYDVPDYALQASGGGGSGGGGSGGGGSASSLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 15 - DNA, 450 bp, synthetic construct
  misc_feature 1..450: note = Cwp2 tether
  source 1..450: mol_type = other DNA
  CDS 1..450
aaggacaatagctcgacgattgaaggtagatacccatacgacgttccagactacgctctgcaggctagtggtggtggtggttctggtggtggtggttctggtggtggtggttctgctagcggatccggtggcggtggatctggaggaggcggttcttggtctcacccacaatttgaaaagggtggagaaaacttgtactttcaaggcggtggtggaggttctggcggaggtggctccggctcagctatctctcaaatcaccgacggtcaaatccaagccactaccacagctaccactgaagctacaactaccgctgctccttcatctactgttgaaactgtttctccatcttccaccgaaaccatctctcaacaaaccgaaaacggtgctgctaaggctgctgttggtatgggtgctggtgctttggctgctgctgctatgttgttgtaa

SEQ ID NO: 16 - protein, 149 aa, synthetic construct
  REGION 1..149: note = Synthetic Construct
KDNSSTIEGRYPYDVPDYALQASGGGGSGGGGSGGGGSASGSGGGGSGGGGSWSHPQFEKGGENLYFQGGGGGSGGGGSGSAISQITDGQIQATTTATTEATTTAAPSSTVETVSPSSTETISQQTENGAAKAAVGMGAGALAAAAMLL

SEQ ID NO: 17 - DNA, 468 bp, synthetic construct
  misc_feature 1..468: note = Ccw12 tether
  source 1..468: mol_type = other DNA
  CDS 1..468
aaggacaatagctcgacgattgaaggtagatacccatacgacgttccagactacgctctgcaggctagtggtggtggtggttctggtggtggtggttctggtggtggtggttctgctagcgctgctaacgttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 18 - protein, 155 aa, synthetic construct
  REGION 1..155: note = Synthetic Construct
KDNSSTIEGRYPYDVPDYALQASGGGGSGGGGSGGGGSASAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 19 - DNA, 510 bp, synthetic construct
  misc_feature 1..510: note = Spi1 tether
  source 1..510: mol_type = other DNA
  CDS 1..510
aaggacaatagctcgacgattgaaggtagatacccatacgacgttccagactacgctctgcaggctagtggtggtggtggttctggtggtggtggttctggtggtggtggttctgctagcttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 20 - protein, 169 aa, synthetic construct
  REGION 1..169: note = Synthetic Construct
KDNSSTIEGRYPYDVPDYALQASGGGGSGGGGSGGGGSASLVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 21 - DNA, 1398 bp, synthetic construct
  misc_feature 1..1398: note = Pst1 tether
  source 1..1398: mol_type = other DNA
  CDS 1..1398
aaggacaatagctcgacgattgaaggtagatacccatacgacgttccagactacgctctgcaggctagtggtggtggtggttctggtggtggtggttctggtggtggtggttctgctagcgctacttcctcttcttccagcataccctcttcctgtaccataagctcacatgccacggccacagctcagagtgacttagataaatatagccgctgtgatacgttagtcgggaacttaactattggtggtggtttgaagactggtgctttggctaatgttaaagaaatcaacgggtctctaactatatttaacgctacaaatctaacctcattcgctgctgattccttggagtccatcacagattctttgaacctacagagtttgacaatcttgacttctgcttcatttgggtctttacagagcgttgatagtataaaactgattactctacccgccatctccagttttacttcaaatatcaaatctgctaacaacatttatatttccgacacttcgttacaatctgtcgatggattctcagccttgaaaaaagttaacgtgttcaacgtcaataacaataagaaattaacctcgatcaaatctccagttgaaacagtcagcgattctttacaattttcgttcaacggtaaccagactaaaatcaccttcgatgacttggtttgggcaaacaatatcagtttgaccgatgtccactctgtttccttcgctaacttgcaaaagattaactcttcattgggtttcatcaacaactccatctcaagtttgaatttcactaagctaaacaccattggccaaaccttcagtatcgtttccaatgactacttgaagaacttgtcgttctctaatttgtcaaccataggtggtgctcttgtcgttgctaacaacactggtttacaaaaaattggtggtctcgacaacctaacaaccattggcggtactttggaagttgttggtaacttcacctccttgaacctagactctttgaagtctgtcaagggtggcgcagatgtcgaatcaaagtcaagcaatttctcctgtaatgctttgaaagctttgcaaaagaaagggggtatcaagggtgaatcttttgtctgcaaaaatggtgcatcatccacatctgttaaactatcgtccacttccaaatctcaatcaagccaaactactgccaaggtttccaagtcatcttctaaggccgaggaaaagaagttcacttctggcgatatcaaggctgctgcttctgcctctagtgtttctagttctggcgcttccagctctagctctaagagttccaaaggcaatgccgctatcatggcaccaattggccaaacaacccctttggtcggtcttttgacggcaatcatcatgtctataatgtaa

SEQ ID NO: 22 - protein, 465 aa, synthetic construct
  REGION 1..465: note = Synthetic Construct
KDNSSTIEGRYPYDVPDYALQASGGGGSGGGGSGGGGSASATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 23 - DNA, 330 bp, synthetic construct
  misc_feature 1..330: note = Aga1/2 tether
  source 1..330: mol_type = other DNA
  CDS 1..330
aaggacaatagctcgacgattgaaggtagatacccatacgacgttccagactacgctctgcaggctagtggtggtggtggttctggtggtggtggttctggtggtggtggttctgctagccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaa

SEQ ID NO: 24 - protein, 109 aa, synthetic construct
  REGION 1..109: note = Synthetic Construct
KDNSSTIEGRYPYDVPDYALQASGGGGSGGGGSGGGGSASQELTTICEQIPSPTLESTPYSLSTTTILANGKAMQGVFEYYKSVTFVSNCGSHPSTTSKGSPINTQYVF

SEQ ID NO: 25 - DNA, 381 bp, synthetic construct
  misc_feature 1..381: note = Aga1/2 tether
  source 1..381: mol_type = other DNA
  CDS 1..381
atgcagttacttcgctgtttttcaatattttctgttattgcttcagttttagcacaggagctgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcgattctcacccctcaacaactagcaaagacagccccataaacacacagtatgtttttaaggacaatagctcgacgattgaaggtagatacccatacgacgttccagactacgctctgcaggctagtggtggtggtggttctggtggtggtggttctggtggtggtggttctgctagc

SEQ ID NO: 26 - protein, 127 aa, synthetic construct
  REGION 1..127: note = Synthetic Construct
MQLLRCFSIFSVIASVLAQELTTICEQIPSPTLESTPYSLSTTTILANGKAMQGVFEYYKSVTFVSNCDSHPSTTSKDSPINTQYVFKDNSSTIEGRYPYDVPDYALQASGGGGSGGGGSGGGGSAS

SEQ ID NO: 27 - DNA, 750 bp, synthetic construct
  misc_feature 1..750: note = Promoter of tdh1 gene
  source 1..750: mol_type = other DNA
agaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttacgctaggaataggatatgcgacgaagacgcttctgcttagtaaccacaccacattttcaaggggtcgatctgcttgcttcctttactgtcacgagcggcccataatcgcgctttttttttaaaatgcgcgagacagcaaacaggaagctcgggtttcaaccttcggagtggtcgcagatctggagactggatccttacaatacagtaaggcaagccaccatctgcttcttaggtgcatgcgacggtatccacgtgcagaacaacatagtctgaagaagggggggaggagcatgttcattctctgtagcactaagagcttggtgataatgaccaaaactggagtctcgaaatcatataaatagacaatatattttcacacaatgtgatttgtagtacagttctactctctctcttgcataaataagaaattcatcaagaacttggtttgatatttcaccaacacacacaaaaaacagtacttcactaaatttacacacaaaacaaa

SEQ ID NO: 28 - DNA, 750 bp, synthetic construct
  misc_feature 1..750: note = Promotoer of hor7 gene
  source 1..750: mol_type = other DNA
acctccatgaaatttttttttttctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaa

SEQ ID NO: 29 - DNA, 750 bp, synthetic construct
  misc_feature 1..750: note = Promoter of hsp150 gene
  source 1..750: mol_type = other DNA
tgtaaaaacaagcaaaaaaaaaccaagaaggaacaaatgcaccaaactgttgatctattctgcaaaaaaagtatggtaaattttttccattatcctggccgctaatccatatggaggtgaacttagaacttgcacaaggatgcgatgaatgataggctttgtgctataattaatgcaggcaggtccgccatgtccaacacgttgctggccgcaaaacgagtcaatctcactgctttgccacgctcatttctccccccttctgcccaattaggcgaccctcacaatgcacatacacatttcccacctctattggaaggggccgtaaatggtaattcttgggagttattcatattaagtgatcttactatttcctatttcggaaattattaaagacaaaaaagctcatttatggctttccgtctgtagtgataagtcgccaactcagcctaatttttcatttctttaccagatcaggaaaactaatagtacaaatgagttttttctcaagcggaacaccacattttgagctaaatttagattttggtcaaaataagaaagatcctaaaaaaggaatggttggtgaaaaatttattagcttgaatggtaggaatcctcgagatataaaaggaacacttgaagtctaacgacaatcaatttcgattatgtccttccttttacctcaaagctcaaaaaaacatcaataagaaactcatattccttttctaaccctagtacaataataataatata

SEQ ID NO: 30 - DNA, 450 bp, synthetic construct
  misc_feature 1..450: note = Promoter of hxt7 gene
  source 1..450: mol_type = other DNA
ccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 31 - DNA, 1000 bp, synthetic construct
  misc_feature 1..1000: note = Promoter of gpm1 gene
  source 1..1000: mol_type = other DNA
tgtggtagaattcaaaagactatgtgatgccataggcaagaagggagactctcactccgagatgggcagcttgatcgcccaggaattgaattgtattgtggtggagaaaggtcagtcagataagatattctcacccgatagtgaaaaagacatgttgacgaacagcgaagagggcagcaacaagagggtaggaggccaaggtgatactttgacaggagctatatcatgcatgcttgcatttagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 32 - DNA, 750 bp, synthetic construct
  misc_feature 1..750: note = Promoter of pgk1 gene
  source 1..750: mol_type = other DNA
acgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 33 - DNA, 1020 bp, synthetic construct
  misc_feature 1..1020: note = Promoter of stl1 gene
  source 1..1020: mol_type = other DNA
tggtctcgcgtgtgaatcagcaatgattctgaaatactccttttacaacctttgcaaagataatgtcattcagtctgatattacgagcgacctggaagcactaatccatattcttcattcaacttactccattttccttggcaaacaatgccccacaatcatatacgtcataactataagggatatgtctggaatgcggccaagatagaattaaagggctgcagaacaccactactgatactcattgccaaggctaggaggcaccatccgtttcattttctttgaggtaagccaatcatgaaatagtatacacatccataacggacgtacggacgaaataagtgccgttgtcccactattccaccgcatttggcccatttggctcactttgactcaacttgcgtcattttaactgatatgaagggtccgactttgtcctttttcggccaccgcataccccacggcgacgcctccgctacctgcatttgagtagcatctccgtttcgcggggtattcggcgctacgtcgcctgttcgagcggctctgttcgttgcatgaaactaaaataagcggaaagtgtccagccatccactacgtcagaaagaaataatggttgtacactgtttctcggctatataccgtttttggttggttaatcctcgccaggtgcagctattgcgcttggctgcttcgcgatagtagtaatctgagaaagtgcagatcccggtaagggaaacacttttggttcacctttgatagggctttcattggggcattcgtaacaaaaaggaagtagatagagaaattgagaaagcttaagtgagatgttttagcttcaattttgtccccttcaacgctgcttggccttagagggtcagaattgcagttcaggagtagtcacactcatagtatataaacaagccctttattgattttgaataattattttgtatacgtgttctagcatacaagttagaataaataaaaaatagaaaaatagaacatagaaagttttagacc

SEQ ID NO: 34 - DNA, 500 bp, synthetic construct
  misc_feature 1..500: note = Terminator of dit1 gene
  source 1..500: mol_type = other DNA
taaagtaagagcgctacattggtctacctttttgttcttttacttaaacattagttagttcgttttctttttctcatttttttatgtttcccctcaaaagttctgattttataatattttatttcacacaattccaattaacagagggggaatagattctttagcttagaaaattagtgatcaatatatatttgcctttcttttcatcttttcagtgatattaatggtttcgagacactgcaatggccctagttgtctaagaggatagatgttactgtcaaagatgatattttgaatttcaattgacgtaattaatgatactattaataatacagagcgtatatgaagtattgcaaataacatgcacagttcttttgggatgagaatgagaatgagaggcgaaggcgggcgttcagaaaagcgttgcggagtaacaagtgattaaatagcacccaaataatcttctttgatactaccgattgcgtgaatagaactcactt

SEQ ID NO: 35 - DNA, 500 bp, synthetic construct
  misc_feature 1..500: note = Terminator of idp1 gene
  source 1..500: mol_type = other DNA
tcgaatttacgtagcccaatctaccactttttttttcattttttaaagtgttatacttagttatgctctaggataatgaactacttttttttttttttactgttatcataaatatatataccttattgatgtttgcaaccgtcggttaattccttatcaaggttccccaagttcggatcattaccatcaatttccaacatcttcatgagttcttcttcttcattaccgtgttttagggggctgttcgcacttctaatagggctatcaccaagctgttctaattcgtccaaaagttcagtaacacgatctttatgcttcagttcgtcataatctttcaattcataaatatttacaatttcgtctacgatattaaattgcctcttgtaggtgcctatcttttccttatgctcttcattttcaccgttttcttgaaaccaaacaccgaactcactacgcatttctttcataggctcatataatacttcttttgacgtcatttg

SEQ ID NO: 36 - DNA, 401 bp, synthetic construct
  misc_feature 1..401: note = Terminator of gpm1 gene
  source 1..401: mol_type = other DNA
gtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaataaccttgatgtttacatcgtagtttaatgtacaccccgcgaattcgttcaagtaggagtgcaccaattgcaaagggaaaagctgaatgggcagttcgaatag

SEQ ID NO: 37 - DNA, 500 bp, synthetic construct
  misc_feature 1..500: note = Terminator of pma1 gene
  source 1..500: mol_type = other DNA
tcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagtttgcttaaaatttcatctcgaattgtcattctaatattttatccacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacactaattt

SEQ ID NO: 38 - DNA, 500 bp, synthetic construct
  misc_feature 1..500: note = Terminator of tdh3 gene
  source 1..500: mol_type = other DNA
gtgaatttactttaaatcttgcatttaaataaattttctttttatagctttatgacttagtttcaatttatatactattttaatgacattttcgattcattgattgaaagctttgtgttttttcttgatgcggtattgcattgttcttgtctttttcgccacatgtaatatctgtaatagatatctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacataaatctagatgcagtaatatacacagattcc

SEQ ID NO: 39 - DNA, 500 bp, synthetic construct
  misc_feature 1..500: note = Terminator of hxt2 gene
  source 1..500: mol_type = other DNA
gagattatacttaaactagcactgatttttttaaggctaatggctactaatactttaatagatgatcttcatacttttttatttaacgatttttaatgatgtttttatttgtaccactcatttatctagatttttttaatactgatcaaatcttacggactcgacgttaaaaagttcctacatacgtctggtacttgaaacgctgcttcgaggtattgacactataagaatacgatccaaatacttacaccgcatgtaaaaatatgccgacaatatgaatacttgttgatgaatgatatttgattttaatccggcaatttacctcctttatataatccaataattgttgataattagtggttaggttgcagtactaataagaattaagacaaatattcttctactatataaaaggtgcaaacaaaacacacgccgatcggccatactaaacaagaccaacataataatggtggaaccatttactgtattttcaatgtaac

SEQ ID NO: 40 - DNA, 500 bp, synthetic construct
  misc_feature 1..500: note = Terminator of ira2 gene
  source 1..500: mol_type = other DNA
aggtgttacataaactaatgaaagaaatatcaatatctatctgtaagcatgaatgtacatatctcatgttagggttttcttatcgctaatttttcgcaatttgttacgtgggttgcttttatacagctacaatttttatatattctatcatgtaatgaatggctcagtaaattcaagcgccacatagactaatgtacataccaatgcatttaattgtaagaatgaaaggggccattcatctaccgtcttagttgaaagtgtttctgtgaattttttcaaattccgttttttcctttttatataatagcatggtggcgcgagcatcttcgactgaagaatgctcaccttcttgaatggaaatttttaaaacctccttggttaatttctttaagctgggtgttttacccttagcatacaacttcctgaatgggaggtgtcttgaagtgtccctgagtagtgactttgggtgggataacatcaatgcttcgagatcatgcttt

SEQ ID NO: 41 - protein, 5 aa, synthetic construct
  REGION 1..5: note = G4S motif
GGGGS

SEQ ID NO: 42 - protein, 457 aa, Penicillium funiculosum
MLRYLSIVAATAILTGVEAQQSVWGQCGGQGWSGATSCAAGSTCSTLNPYYAQCIPGTATSTTLVKTTSSTSVGTTSPPTTTTTKASTTATTTAAASGNPFSGYQLYANPYYSSEVHTLAIPSLTGSLAAAATKAAEIPSFVWLDTAAKVPTMGTYLANIEAANKAGASPPIAGIFVVYDLPDRDCAAAASNGEYTVANNGVANYKAYIDSIVAQLKAYPDVHTILIIEPDSLANMVTNLSTAKCAEAQSAYYECVNYALIKPHLAHVAMYIDAGHAGWLGWSANLSPAAQLFATVYKNASAPASLRGLATNVANYNAWSISSPPSYTSGDSNYDEKLYINALSPLLTSNGWPDAHFIMDTSRNGVQPTKQQAWGDWCNVIGTGFGVQPTTNTGDPLEDAFVWVKPGGESDGTSNSSATRYDFHCGYSGALQPAPEAGTWFQAYFVQLLTNANPALV

SEQ ID NO: 43 - protein, 408 aa, Rasamsonia emersonii
MVRLSPVLLASIAGSGLPLAQAAGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 44 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 45 - protein, 291 aa, Thermomyces lanuginosus
MRSSLVLFFLSAWTALARPVRRAVPQDLLDQFELFSQYSAAAYCAANNHAPVGSDVTCSENVCPEVDAADATFLYSFEDSGLGDVTGLLALDNTNKLIVLSFRGSRSVENWIANLAADLTEISDICSGCEGHVGFVTSWRSVADTIREQVQNAVNEHPDYRVVFTGHSLGGALATIAAAALRGNGYNIDVFSYGAPRVGNRAFAEFLTAQTGGTLYRITHTNDIVPRLPPRDWGYSHSSPEYWVTSGNDVPVTANDITVVEGIDSTDGNNQGNIPDIPSHLWYFGPISECD

SEQ ID NO: 46 - protein, 146 aa, Sus scrofa
MKFLVLAVLLTVGAAQEGISSRALWQFRSMIKCAIPGSHPLMDFNNYGCYCGLGGSGTPVDELDRCCETHDNCYRDAKNLDSCKFLVDNPYTESYSYSCSNTEITCNSKNNACEAFICNCDRNAAICFSKAPYNKEHKNLDTKKYC

SEQ ID NO: 47 - protein, 150 aa, Streptomyces vialaceoruber
MRTTTRTRTTLAAVGAALALGVAAAPPQAAPADKPQVLASFTQTSASSQNAWLAANRNQSAWAAYEFDWSTDLCSQAPDNPFGFPFNTACARHDFGYRNYKAAGSFDANKSRIDSAFYEDMKRVCTGYTGEKNTACNSTAWTYYQAVKIL

SEQ ID NO: 48 - protein, 295 aa, Aspergillus niger
MFSLARLGTVAGLFLLAQAAPASLRRDVSSSLLNNLDLFAQYSAAAYCDENLNSTGTKLTCSVGNCPLVEAASTQSLDEFNESSSYGNPAGYLAADETNKLLVLSFRGSADLANWVANLNFGLEDASDLCSGCEVHSGFWKAWSEIADTITSKVESALSDHSDYSLVLTGHSYGAALAALAATALRNSGHSVELYNYGQPRLGNEALATYITDQNKGGNYRVTHTNDIVPKLPPTLLGYHHFSPEYYISSADEATVTTTDVTEVTGIDATGGNDGTDGTSIDAHRWYFIYISECS

SEQ ID NO: 49 - protein, 515 aa, synthetic construct
  REGION 1..515: note = Mutated glucoamylase
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 50 - protein, 652 aa, synthetic construct
  REGION 1..652: note = Mutated alpha-amylase
MLLQAFLFLLAGFAAKISAGPAAANAETANKSNNVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 51 - protein, 713 aa, synthetic construct
  REGION 1..713: note = Mutated maltogenic alpha-amylase
MLLQAFLFLLAGFAAKISAYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 52 - protein, 19 aa, Saccharomyces cerevisiae
MLLQAFLFLLAGFAAKISA

SEQ ID NO: 53 - protein, 22 aa, synthetic construct
  REGION 1..22: note = HA tag
KDNSSTIEGRYPYDVPDYALQA

SEQ ID NO: 54 - protein, 18 aa, synthetic construct
  REGION 1..18: note = Modified G4S linker
SGGGGSGGGGSGGGGSAS

SEQ ID NO: 55 - DNA, 750 bp, synthetic construct
  misc_feature 1..750: note = Promoter of trx2 gene
  source 1..750: mol_type = other DNA
ataaccacaatgatcgcagaccgtcgacatgataatgacttctttaaagtgtgggatatttactggcttcatatgagtttcacactcttgggtacacgatggacaagaagctctgaatgtttgcacctcgttgttaaagttttcgatgtcggtggcgtctgacaaaaactgtgggtttgctgagccaactttgacagattcagaaggatttctttcacggttggccaattgttttaattgttcttggcgacgttgctccaactggtctctagtaataatgccaacttgaacgttttgttcgtcggttctcacgtactgggtatgagaccatttgtgttgaggttcaccgggtttgtattcgatccaggaatttcccgcaggatcatccaaaataaatgtaatcggaatagtgttgggttcacaattgatgtaggatttaactttctgtacgaagtcatcgatcttcttgtaaagagcttcgtctatagattttctcatttcctggtcttgcgacagatcgtcgatcatctcggataacaaaccttcaactgtcgtcaattgacctctcttagcaggaatctcaatgtctagctcgacaaacttacaagtggcagtttctgacttaataacttgcctgttaaaatcttcacggcactccactttcaaaacgtaacgagagcccttctcttgaatttgagaagcgggttggatctcacagttcttaaacccacagtgaggacagtcgaatga

SEQ ID NO: 56 - DNA, 750 bp, synthetic construct
  misc_feature 1..750: note = Promoter of sti1 gene
  source 1..750: mol_type = other DNA
taccacaaagggtaaaacgtcaataaaagcaagcaatgttattcgtgccagagaactatatctggcgtacgtcttatatataacacaagcaagggcaaatacgtggttagtggtgtaaaaactgaagggattaatttacatacaactagatccatctttctcaaaaatgacttcagtatcgccctcgccacctgccagtcgatcgggctcaatgtgctccgacttaccgtcctctttgcagactgagaaactggcacatattataggtcttgatgccgacgatgaagttctccggcgcgtaaccaagcagttgagcagatctaggagaattgcttgtctgactggggcaggcatttcgtgcaacgcgggcattcctgactttcgctcttctgatgggctctacgacctagtgaaaaaggattgttcacagtattggtctatcaagtccggcagggaaatgtttgatatttcgctatttagagatgacttcaaaatatccatttttgctaaatttatggagaggctctattcaaatgttcaattggcaaagccgactaagacgcacaagttcattgcgcatctaaaagataggaacaaactgctgcgctgttacacgcaaaacatcgatgggctcgaagaaagcataggacttactttatcaaataggaaattaccgcttacctcatttagttcacattggaaaaatctggatgtcgttcagttgcacggcgacctgaaaactctttcgtg

SEQ ID NO: 57 - DNA, 750 bp, synthetic construct
  misc_feature 1..750: note = Promoter of gdp1 gene
  source 1..750: mol_type = other DNA
aaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaactccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaagaaacaattgtatattgtacacccccccccctccacaaacacaaatattgataatataaag

SEQ ID NO: 58 - DNA, 750 bp, synthetic construct
  misc_feature 1..750: note = Promoter of cup1 gene
  source 1..750: mol_type = other DNA
taaatatatgctgagatttagcaagaaaaaacaaaattttgcgagaaactctgataacaataatgttacagattatagtcagtcggcgaagaacaaaaatgttctcttgaaattccccgttagtgaactgaacagaatctatttaaaatttaaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 59 - DNA, 750 bp, synthetic construct
  misc_feature 1..750: note = Promoter of glo1 gene
  source 1..750: mol_type = other DNA
tattaatccaaaaagtgaatctttgacacccctttctcctatcttgcttagtaccgagtgatcttttgcataatcaaaacaagtgaagaaggtcgagttttgaaccttctctaatataagtcgacggtctttcgtgatggcgtcaatattacttagtcttttgtacttcacatataccattaatggcgtcaatcccttataattttcctcattaatatctagcttagtcagttgtaaaaggattggaatgtttgttttcaaaacatgcaacaagctattgcccttattatcggtgtggtctagatagtcaaacaaactgttgtgctttctataccaagtatttgcgatatcaaatgcggtcttcaccatttcttcataatttggttgatcataacttctaaagatggaaaataaaggcgtttgtccactggagttttttcgtttccaatcaatataatttccgatgcttttaagtatatccatttcatgggttagataatgagccacggtacgtgcatatttatctgttttattgatataagaaaccagctcctcttcagtacaattgaacagcatgatctttatgagaactttcgcggcttctaaatttcctgcttttatcgattctataagcagcgtagaaccatcaatcgtttcgtcctctaaaaggtcttctactggaaagtcgttttcatattcagaaagaatatctaataagatgtagtttttatgatttgttatacatatgga

SEQ ID NO: 60 - DNA, 750 bp, synthetic construct
  misc_feature 1..750: note = Promoter of ctt1 gene
  source 1..750: mol_type = other DNA
ttaactgcaaagacccgtcacctatgaataagatgactctcttgttggggtcaatctcctcagcggcaaaggcagcacctaaagttgctcctgttgtaaaaccgatagacccccacaacacttgcgagataccgtaggcgtccttaggaaagatagtttgattgataccgaaggcagacgtgccggtctcggaaatgataacatcaccttcttgcaagaacttggacaattcgttccacaaccactcttgtttcaagggcgtgctagcagatacacctttgtttgcgggagttttggttggtacgggaacgctcttgtagcccttaataacatcgggaataaccttcagtaagttttgtagtgcaaatttcatttgtacgccggggaacgtagcgttcttcacctttacgtaatcggaatgaaactccactacatttttagtcttgtaggagtaggaaaacgaacctgtgttaaaatcagagagcaaagcaccgaccgaaaggatcaaatcagccgactcaacggcctgtttcacgtctggtttggacagcgttcccacataaacaccgccatatctgggatgctgttcatctattgaccctttacctaaaggtgtcacaaaagctgggaattgcgtcaaatcaattaacttctgggtttcctttttaacgttgtgcctagaagcacaggcatccgatagtatgacagggtttttcgaattctggatcaattctagtacggtatcaataa

SEQ ID NO: 61 - DNA, 750 bp, synthetic construct
  misc_feature 1..750: note = Promoter of ygp1 gene
  source 1..750: mol_type = other DNA
tctcataattggtcagaagaaaggaaatctaggcaattgagttctttgggtaaaaaagagtcacaatttttaaggctgcgtagaacgagattatcgttagaagatttccacactgtgaaagtcattggaaaaggggcatttggtgaagtcagactggttcagaagaaagacaccggaaaaatatatgcaatgaaaactttattaaaatccgaaatgtacaaaaaggaccaattagcacacgtcaaggctgaaagggatgttctggctggaagtgattctccatgggtggtttcgctatattactcattccaagatgctcaatacttatatttaatcatggaatttttgcccggtggtgatttgatgaccatgctaatcaggtggcaactatttacagaggatgttactagattttacatggctgagtgtattttggccatcgaaaccattcataaattaggattcattcacagagatattaaaccagataatattttgatcgatattagaggtcatataaaattatctgatttcgggttgtctacggggttccataaaactcacgactcaaactactataaaaagcttttgcaacaagatgaggcaactaatggtatttccaagccaggtacttacaatgcaaatacaaccgatactgcaaataaaaggcaaacaatggttgtggattctattagtctaacaatgtcaaacaggcaacaaattcagacatggagaaaatcacgtcgttta

SEQ ID NO: 62 - DNA, 750 bp, synthetic construct
  misc_feature 1..750: note = Promoter of gsy2 gene
  source 1..750: mol_type = other DNA
tattatagctcttgataacaatgaaatttcaacaattattcactcaaccctcaagtgtatatcgccaagatcctaatcacccctctctggtggccttccctcattgtttttacacagaacaagaatccataaaatcaagatattgggaacaattcattgtcttaggaacaagtttttataatttagtttcattagcctattgtaacttcttgtttttttttttattttggctggcaaaacaagaagtataaagaagttaccactgctataacgtatatcttaaatcaagtgggcaattcttcgaataaacttagaggcgctgaagaaagaaccgctcaattagttcgatcatacaaaatacagatattgcgctgtgtattatcattacagaaagtgtgatataaacaatgctgtgttgtattcggtcttttttttccgcctgcagattgtcgtttttgtttttttttctcgtgtagctgcagttatcatcgttttgcatgtctgttctgtcggtgaagccgatctcatggcacttcgctttgataccgtaaagaatcttcttcattaatacggcactgcaaggcgatatctatctctggggttaggactgataaataggtacttatcagttatcactagagacgaactgtttacgttttgaagctcgacagcaggggttcttaacgggcgtcggcctagacctcaccttgcgagaaatttttagttcatatccaggggatggtgaatggt

SEQ ID NO: 63 - DNA, 750 bp, synthetic construct
  misc_feature 1..750: note = Promoter of hsp12 gene
  source 1..750: mol_type = other DNA
gcttgtccacaatcaccatggggttatattgcattatttttttatcttcgttatcattccatactgtggcttcattccagtattttatgagtttgctgaaggattcgggggtaatagtgcccacggtggtcaaaacctttagcgtctcagtgacctgttccaaatccccctcttccattcgccttatataaaatccatcgggtaagctcattattttatgttgatatccagcttccgttcgtcaactattctctcgagctcagttttgggttttggcatgtaaacaatgccttcgcttgtattacgcaaaaaaaaaaaaaataaataaaaaaaaaaaaaaaaaaataaggtataaatcgttggttcttttatgcacaattatttaactatagttatctatttacgtaaaggcttctatttttccttatctacaagaaattgcatgaagtttaattttttttgtcacctttgatcttcctgaatgtgttggtaatgaaattttctagtctcttcaaagtgttgtcgtcatccttttttgcgtgtttttcctcttcttgtttttgtttcttcagtatctcttcattaatggctgtacccgcggctgtgcccggttgcatggttttcttggccatgtcatcgttggtgacatctgccagtgcctcccacaagcatttttctgccagcgattgcggtttcttaacaacgatcttgtactggactttcatatcaccgcgaatggttgatgtttta

SEQ ID NO: 64 - DNA, 750 bp, synthetic construct
  misc_feature 1..750: note = Promoter of mol1/thi4 genes
  source 1..750: mol_type = other DNA
tcgttgaaagttctagatttgggtacggaagcagccatgtaaatggtcttcctttgtaataatatcgtcactggagtttttaaattagagtacggattcaagttaacgaaaaatttctgttggtatctgatagcaatttgtcgacaaggtggagagtttttgaagtttttttttgtaacagttatgttacactatttaccagagaaacaccaagacaggagccctcagaagctttcgtgttgtcagaacataaatatcttctatatgttttattcttttatattaatcaatattttatgttaaatcatattattattcgttaatgcttaacttgccctatttcaaaatcggaagggtagtatggtgacaagcggtgtttttggtgtatgtttactgctaagagtataccgtcctgtttacttttttaccttttgttccgttattgatttttcgcagcaaggttgaagcttccatatttgaaaatgaaattaagaagtagagatgaaaaacagcgacgaaattactttttatagcttctttcccttctgactccgttttataaacaggggatctttaagggcttacaaaataaatataaacactaacacatatatattattcgacctacagtttttgcgaccaatgaagtttacctattttgagatggtgcttacgtagtttgcttaaggtcaaggcgggattaagcttgcgctttctctaaataatatcatatgttacagacagggataa

SEQ ID NO: 65 - protein, 695 aa, Geobacillus stearothermophilus
MYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 66 - protein, 411 aa, Citrobacter braakii
EEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRTLENVLNFSQSETCKTTEKSTKCTLPEALPSEFKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLKWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 67 - protein, 410 aa, Escherichia coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 68 - protein, 19 aa, Saccharomyces cerevisiae
MLLQAFLFLLAGFAAKISA

SEQ ID NO: 69 - protein, 18 aa, Saccharomyces cerevisiae
MQLLRCFSIFSVIASVLA

SEQ ID NO: 70 - DNA, 500 bp, Saccharomyces cerevisiae
  source 1..500: mol_type = other DNA
tagcgtgttacgcacccaaactttttatgaaagtctttgtttataatgatgaggtttataaatatatagtggagcaaagattaatcactaaatcaagaagcagtaccagtattttttttatatcaagtagtgataatggaaatagcccaaatttggcttccgtcggcacatagcacgtttgagagacattatcaccatcaagcatcgagccgcccaaacctaactgtataagttttttcacgtttttgatttttccttgcacacttcgatattactctcacgataaaagggccgaagagaatatttttcttgaacatccagaattttaattcggagaaatttcacaagccgccgatttaagggtcctgtgttcttaataatcagcctctctcaaagcaggtaagaggcagtctttcttttaacaataggagacattcgaactaaaacatcagccccaaaaatgcgcttgaaggtcattaggatttggatttcttcctcat

SEQ ID NO: 71 - protein, 460 aa, Pyrococcus furiosus
MNIKKLTPLLTLLLFFIVLASPVSAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 72 - protein, 457 aa, Thermococcus hydrothermalis
MARKVLVALLVFLVVLSVSAVPAKAETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 73 - DNA, 390 bp, synthetic construct
  misc_feature 1..390: note = SPI1 tethering moiety
  source 1..390: mol_type = other DNA
  CDS 1..390
ttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 74 - protein, 129 aa, synthetic construct
  REGION 1..129: note = Synthetic Construct
LVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 75 - DNA, 66 bp, synthetic construct
  misc_feature 1..66: note = 21aa-truncation of the SPI1 tethering moiety
  source 1..66: mol_type = other DNA
  CDS 1..66
gctgctaacgcaagagcaatcccaggagccctaggtttggctggtgcagttatgatgcttttatga

SEQ ID NO: 76 - protein, 21 aa, synthetic construct
  REGION 1..21: note = Synthetic Construct
AANARAIPGALGLAGAVMMLL

SEQ ID NO: 77 - DNA, 156 bp, synthetic construct
  misc_feature 1..156: note = 51aa-truncation of the SPI1 tethering moiety
  source 1..156: mol_type = other DNA
  CDS 1..156
ttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 78 - protein, 51 aa, synthetic construct
  REGION 1..51: note = Synthetic Construct
LTITNCPCTIEKPTSETSVSSTHDVETNSNAANARAIPGALGLAGAVMMLL

SEQ ID NO: 79 - DNA, 246 bp, synthetic construct
  misc_feature 1..246: note = 81aa-truncation of the SPI1 tethering moiety
  source 1..246: mol_type = other DNA
  CDS 1..246
gaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 80 - protein, 81 aa, synthetic construct
  REGION 1..81: note = Synthetic Construct
EVVSEFTTYCPEPTTFVTNGATFTVTAPTTLTITNCPCTIEKPTSETSVSSTHDVETNSNAANARAIPGALGLAGAVMMLL

SEQ ID NO: 81 - DNA, 336 bp, synthetic construct
  misc_feature 1..336: note = 111aa-truncation of the SPI1 tethering moiety
  source 1..336: mol_type = other DNA
  CDS 1..336
attgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 82 - protein, 111 aa, synthetic construct
  REGION 1..111: note = Synthetic Construct
IAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 83 - DNA, 339 bp, synthetic construct
  misc_feature 1..339: note = CCW12 tethering moiety
  source 1..339: mol_type = other DNA
  CDS 1..339
gttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 84 - protein, 112 aa, synthetic construct
  REGION 1..112: note = Synthetic Construct
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 85 - DNA, 75 bp, synthetic construct
  misc_feature 1..75: note = 24aa-truncation of the CCW12 tethering moiety
  source 1..75: mol_type = other DNA
  CDS 1..75
tacactggtgctgctgctaaggctttgccagctgctggtgctttgttggctggtgccgctgctttgttgttgtaa

SEQ ID NO: 86 - protein, 24 aa, synthetic construct
  REGION 1..24: note = Synthetic Construct
YTGAAAKALPAAGALLAGAAALLL

SEQ ID NO: 87 - DNA, 150 bp, synthetic construct
  misc_feature 1..150: note = 49aa-truncation of the CCW12 tethering moiety
  source 1..150: mol_type = other DNA
  CDS 1..150
actgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 88 - protein, 49 aa, synthetic construct
  REGION 1..49: note = Synthetic Construct
TAAPVTSTEAPKNTTSAAPTHSVTSYTGAAAKALPAAGALLAGAAALLL

SEQ ID NO: 89 - DNA, 225 bp, synthetic construct
  misc_feature 1..225: note = 74aa-truncation of the CCW12 tethering moiety
  source 1..225: mol_type = other DNA
  CDS 1..225
accgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 90 - protein, 74 aa, synthetic construct
  REGION 1..74: note = Synthetic Construct
TVDDVITQYTTWCPLTTEAPKNGTSTAAPVTSTEAPKNTTSAAPTHSVTSYTGAAAKALPAAGALLAGAAALLL

SEQ ID NO: 91 - DNA, 300 bp, synthetic construct
  misc_feature 1..300: note = 99aa-truncation of the CCW12 tethering moiety
  source 1..300: mol_type = other DNA
  CDS 1..300
gtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 92 - protein, 99 aa, synthetic construct
  REGION 1..99: note = Synthetic Construct
VTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 93 - protein, 15 aa, synthetic construct
  REGION 1..15: note = Linker 1
GGGGSGGGGSGGGGS

SEQ ID NO: 94 - protein, 8 aa, synthetic construct
  REGION 1..8: note = Linker 2
GGGGGGGG

SEQ ID NO: 95 - protein, 40 aa, synthetic construct
  REGION 1..40: note = Linker 3
GGGGSGGGGSGGGGSGGGGSGGGGSGGGGSGGGGSGGGGS

SEQ ID NO: 96 - protein, 12 aa, synthetic construct
  REGION 1..12: note = Linker 4
GSAGSAAGSGEF

SEQ ID NO: 97 - protein, 12 aa, synthetic construct
  REGION 1..12: note = Linker 5
EAAKEAAKEAAK

SEQ ID NO: 98 - protein, 20 aa, synthetic construct
  REGION 1..20: note = Linker 6
APAPAPAPAPAPAPAPAPAP

SEQ ID NO: 99 - protein, 46 aa, synthetic construct
  REGION 1..46: note = Linker 7
AEAAAKEAAAKEAAAKEAAAKALEAEAAAKEAAAKEAAAKEAAAKA

SEQ ID NO: 100 - protein, 4 aa, synthetic construct
  REGION 1..4: note = EA2K motif
EAAK

SEQ ID NO: 101 - protein, 5 aa, synthetic construct
  REGION 1..5: note = EA3K motif
EAAAK

SEQ ID NO: 102 - DNA, 1752 bp, Aspergillus niger
  source 1..1752: mol_type = other DNA
tccaatggtattgaggcttccttgttgactgatccaaaagatgtttctggtagaaccgtcgattacattattgctggtggtggtttgactggtttaactactgctgctagattgactgaaaacccaaacatctccgttttggttatcgaatctggttcctacgaatctgatagaggtccaattatcgaagatttgaacgcctacggtgatatctttggttcttctgttgatcatgcttacgaaaccgttgaattggctaccaacaatcaaactgccttgattagatctggtaacggtttaggtggttctactttggttaatggtggtacttggactagaccacataaggctcaagttgattcttgggaaactgtttttggtaacgaaggttggaattgggataatgttgctgcttattcattgcaagctgaaagggctagagcaccaaatgctaaacaaattgctgctggtcattacttcaatgcctcttgtcatggtgttaacggtactgttcatgctggtccaagagatactggtgatgattattctccaatcgttaaggctttgatgtccgctgttgaagatagaggtgttccaactaagaaagatttcggttgtggtgatccacatggtgtttctatgtttccaaataccttgcacgaagatcaagttagatctgatgctgcaagagaatggttgttgccaaattatcaaagaccaaacttgcaggtcttgactggtcaatatgttggtaaggttttgttgtcccaaaacggtactactccaagagctgttggtgttgaatttggtactcataagggtaacacccataacgtttacgccaaacatgaagttttgttagctgctggttctgctgtttctccaactattttggaatactccggtatcggtatgaagtccattttggaaccattgggtattgataccgttgttgatttgccagttggtttgaacttacaagatcaaactaccgctaccgtcagatctagaattacttctgctggtgctggtcaaggtcaagctgcttggtttgctacttttaacgaaactttcggtgactactctgaaaaggcccatgaattattgaacaccaaattggaacaatgggctgaagaggctgttgctagaggtggttttcataacactactgccttgttgatccagtacgaaaattacagagattggatcgttaaccacaacgttgcttactctgagttgtttttggatactgctggtgttgcttcttttgatgtatgggatttgttgccattcaccagaggttacgttcacattttggataaggatccatacttgcatcatttcgcttacgacccacaatactttttgaacgaattggacttgttgggtcaagccgctgctactcaattggctagaaacatttctaattccggtgctatgcaaacctatttcgctggtgaaactattccaggtgataacttggcttatgatgctgatttgtctgcttggactgagtacattccataccatttcagaccaaattaccacggtgttggtacttgttctatgatgccaaaagaaatgggtggtgttgttgataatgcagctagagtttacggtgttcaaggtttgagagttatcgatggttctattccaccaactcaaatgtcctctcatgttatgactgttttctacgctatggccttgaagatttccgatgcaattttggaagattacgcctccatgcaataa

SEQ ID NO: 103 - protein, 583 aa, Aspergillus niger
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 104 - DNA, 1437 bp, Aspergillus oryzae
  source 1..1437: mol_type = other DNA
gctactccagctgattggagatcacaatctatctactttttgttgaccgacagattcgctagaactgatggttctactactgctacttgtaataccgctgatagaaagtattgtggtggtacttggcaaggtatcatcgataagttggattacattcaaggtatgggtttcaccgctatttggattactccagttactgctcaattgccacaaactactgcttatggtgatgcttatcatggttattggcaacaggatatctactccttgaacgaaaattacggtactgccgatgatttgaaggctttgtcatctgctttacatgaacgtggtatgtacttgatggttgatgttgttgctaaccacatgggttatgatggtgctggttcttctgttgattactctgtttttaagcccttcagctcccaagattactttcatccattctgcttgatccaaaactacgaagatcaaactcaagtcgaagattgctggttgggtgataatactgtttctttgccagatttggataccaccaaggatgttgttaagaacgaatggtatgattgggtcggttctttggtttccaactactctattgatggtttgagaatcgataccgtcaagcacgttcaaaaagatttttggccaggttacaacaaagctgctggtgtttactgtattggtgaagttttagatggtgatccagcttacacttgtccataccaaaatgttatggatggcgttttgaactacccaatctactacccattattgaacgctttcaagtctacctctggttctatggatgacttgtacaacatgatcaacaccgttaagtctgattgtccagattctactttgttgggtactttcgttgaaaaccacgataatccaagattcgcttcttacaccaacgatattgctttggctaaaaacgttgccgccttcattattttgaacgatggtattccaattatctacgccggtcaagaacaacattatgctggtggtaatgatccagcaaatagagaagctacttggttgtctggttatccaactgattccgagttgtacaaattgattgcttccgctaacgccattagaaactacgctatttctaaggatactggcttcgttacttacaagaattggcctatctacaaggatgataccactattgctatgagaaagggtacagatggttctcaaatcgttaccatcttgtctaacaaaggtgcttctggtgattcctacactttgtctttgtctggtgcaggttatactgctggtcaacaattgactgaagttattggttgtactaccgttaccgttggttctgatggtaatgttcctgttccaatggctggtggtttgccaagagtcttgtatccaacagaaaaattggccggttccaagatctgttcttcttcttga

SEQ ID NO: 105 - protein, 478 aa, Aspergillus oryzae
ATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 106 - DNA, 63 bp, synthetic construct
  misc_feature 1..63: note = Signal peptide from fungal amylase from Aspergillus oryzae
  source 1..63: mol_type = other DNA
atgatggttgcttggtggtctttgttcttgtacggtttacaagttgctgctccagctttggct

SEQ ID NO: 107 - protein, 21 aa, synthetic construct
  REGION 1..21: note = Signal peptide from fungal amylase from Aspergillus oryzae
MMVAWWSLFLYGLQVAAPALA

SEQ ID NO: 108 - protein, 687 aa, Geobacillus stearothermophilus
MSSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN